(19) United States
(12) Patent Application Publication
(10) Pub. No.: US 2026/0183345 A1
(43) Pub. Date: Jul. 2, 2026

(54) METHODS OF ELECTROSPRAY DRYING ANAEROBIC BACTERIA AND COMPOSITIONS THEREOF

(71) Applicant: AXIOTA U.S., INC., Fort Collins, CO (US)

(72) Inventors: Ming Woei LAU, Topeka, KS (US); James Scott DROUILLARD, Olsburg, KS (US); Amanda PETERSON, Wamego, KS (US); Celine Caroline APERCE, Wamego, KS (US); Gina Rae HERREN, Wamego, KS (US)

(21) Appl. No.: 19/099,726

(22) PCT Filed: Jul. 28, 2023
(86) PCT No.: PCT/US2023/028982

§ 371 Date: Jan. 29, 2025

Related U.S. Application Data
(60) Provisional application No. 63/369,915, filed on Jul. 29, 2022.

(51) Int. Cl. A61K 35/74; A23K 10/16; A23K 40/10; A23K 50/10; A61K 9/00; A61K 9/14; A61K 9/16; B05B 5/16; C12N 1/20; C12R 1/01

(52) U.S. Cl. CPC A61K 35/74; A23K 10/16; A23K 40/10; A23K 50/10; A61K 9/0053; A61K 9/141; A61K 9/1623; A61K 9/1652; A61K 9/1682; B05B 5/1608; C12N 1/20; C12R 2001/01

(57) ABSTRACT

The present disclosure relates to methods and systems of electrospray drying anaerobic bacterial cells and/or Megasphaera elsdenii, compositions and feed additives thereof. The methods, compositions, and feed additives thereof will result in improved viability and yield after extended storage of the bacteria. Such improvements are beneficial for a range of applications, including uses in animal health and feeds.

### CROSS-REFERENCE TO RELATED APPLICATIONS

This application claims the priority benefit of U.S. Provisional Appl. No. 63/369,915, filed Jul. 29, 2022, which is herein incorporated by reference in its entirety.

### BACKGROUND OF THE DISCLOSURE

### Field of Disclosure

The present disclosure relates to methods and systems for electrospray drying anaerobic bacteria into a dried powder by applying low amounts of heat.

### Background

Megasphaera elsdenii (i.e., M. elsdenii) is an anaerobic, non-motile, and Gram-negative diplococci that utilizes lactate as a preferred carbon source and can help to prevent acidosis, which is a common digestive disorder that affects millions of beef and dairy cattle each year.

When cattle and other ruminants ingest large quantities of starchy foods (e.g., cereal grains) or simple sugars, opportunistic microorganisms in the stomach can rapidly ferment these compounds into lactic acid. Lactic acid is a potent organic acid and can lead to lactic acidosis, which can disrupt normal digestive activity and cause extensive damage to the digestive tract lining in ruminants. Affected animals have suboptimal performance. And, the most acute form of lactic acidosis can cause irreversible damage to an animal's digestive and respiratory systems, as well as increased mortality rates.

M. elsdenii can help to control lactic acidosis by converting lactic acid into volatile fatty acids (VFA; e.g. butyrate, propionate, and acetate), which are harmless organic compounds. However, M. elsdenii populations in the gastrointestinal tract of ruminants are often at levels too low to prevent the risk of acidosis. Accordingly, a liquid culture of live cells from a strain of M. elsdenii, Lactipro®, was developed to increase the rate of colonization of M. elsdenii in the gastrointestinal tract of ruminants. See, e.g., U.S. Pat. No. 7,550,139. However, there are practical constraints that have limited the use of products containing M. elsdenii, including the difficulty of maintaining M. elsdenii products under the anaerobic conditions required by the organism and the difficulty of transporting the M. elsdenii products from production facility to site of end-use within 14 days, after which time the viability of M. elsdenii in the product decreases significantly. A product containing freeze-dried M. elsdenii (Lactipro NXT©) has a longer shelf life than Lactipro®, but there is a desire for further improved methods for scaling up production and rendering it more cost effective. See WO 2018/144653 A1, which is herein incorporated by reference in its entirety.

Methods such as spray drying typically use a heated drying fluid (e.g., heated air) to produce the dried powder, which can result in a reduction of stability and/or viability of anaerobic bacteria (e.g., M. elsdenii).

Therefore, there is a need for new methods and apparatus for carrying out the spray drying process, which increase the stability and viability of anaerobic bacteria (e.g., M. elsdenii) and which reduce or eliminate the disadvantageous characteristics of the conventional spray drying process. There is also a need for stable powder formulations of other anaerobic bacteria, including Bifidobacterium, such as B. breve, Lactobacillus, such as L. plantarum, Bifidobacterium, such as B. animalis subsp. lactis, Pediococcus, such as P. acidilactici, Lactobacillus, such as L. casei, Fibrobacter, such as F. succinogenes, and Butyrivibrio, such as B. fibrisolvens, as well as methods of producing the same, that overcome existing limitations. There is also a need for stable powder formulations of other anaerobic bacteria, including Bifidobacterium, such as B. breve, Lactobacillus, such as L. plantarum, Bifidobacterium, such as B. animalis subsp. lactis, Pediococcus, such as P. acidilactici, Lactobacillus, such as L. casei, Fibrobacter, such as F. succinogenes, and Butyrivibrio, such as B. fibrisolvens, Ruminococcus, such as R. flavefaciens, as well as methods of producing the same, that overcome existing limitations.

### BRIEF SUMMARY OF THE DISCLOSURE

In some aspects, provided herein is a system for spray drying M. elsdenii cells into a dried powder, the system comprising a source of water, a carrier, and M. elsdenii cells; a tank comprising a stirring device and the tank is arranged to receive the water, the carrier, and the M. elsdenii cells from the source to form a slurry having a viscosity in the range of about 1 cP to about 500 cP; an electrode configured to apply an electrostatic charge to the slurry using a voltage gradient or oscillating voltages; an atomizer; a drying chamber comprising an inlet end, an outlet end, and an internal volume located between the inlet end and the outlet end, the internal volume being configured to hold the slurry and a drying fluid. In some aspects, the drying chamber is configured to dry the slurry. In some aspects, the atomizer is configured to: (i) receive the slurry from the tank; and (ii) discharge the slurry into the drying chamber for contact with the drying fluid to form the dried powder containing the M. elsdenii cells. In some aspects, the drying fluid comprises nitrogen or argon at a temperature between about 50° C. to about 100° C., and wherein the entire system is under less than 2% oxygen.

In some aspects, provided herein is a method of electrospray drying M. elsdenii cells into a dried powder, comprising: preparing a culture comprising M. elsdenii cells, a growth media, and an osmolyte protectant molecule; harvesting the M. elsdenii cells forming a slurry comprising water, a carrier, and the M. elsdenii cells, wherein the carrier has a final concentration of about 1% to about 50% wt and the slurry has a viscosity in the range of about 1 cP to about 500 cP; applying an electrostatic charge to the slurry using a voltage gradient or oscillating voltages; atomizing the slurry to generate a spray of liquid droplets of the slurry; introducing the spray of liquid droplets of the slurry into a drying chamber; and feeding a drying fluid at a temperature between 50° C. to 100° C. into the drying chamber to dry the liquid droplets to form the dried powder comprising a plurality of dried particles containing the M. elsdenii cells encapsulated within the carrier. In some aspects, the dried powder comprises less than 15% moisture content. In some aspects, the entire method is performed under less than 2% oxygen.

In some aspects, provided herein is a method of electrospray drying M. elsdenii cells into a dried powder, comprising: preparing a culture comprising M. elsdenii cells, a growth media, and an osmolyte protectant molecule, wherein during the culture the pH, temperature, and/or osmolality are altered for a time period; harvesting the M. elsdenii cells; forming a slurry comprising water, a carrier, and the M. elsdenii cells, wherein the carrier has a final concentration of about 1% to about 50% wt and the slurry has a viscosity in the range of about 1 cP to about 500 cP; applying an electrostatic charge to the slurry using a voltage gradient or oscillating voltages; atomizing the slurry to generate a spray of liquid droplets of the slurry; introducing the spray of liquid droplets of the slurry into a drying chamber; and feeding a drying fluid at a temperature between about 50° C. to about 100° C. into the drying chamber to dry the liquid droplets to form the dried powder comprising a plurality of dried particles containing the M. elsdenii cells encapsulated within the carrier. In some aspects, the dried powder comprises less than 15% moisture content. In some aspects, the entire method is performed under less than 2% oxygen.

In some aspects, the carrier has a final concentration of about 2% to about 30% wt.

In some aspects, provided herein is a method of electrospray drying M. elsdenii cells into a dried powder, comprising: preparing a culture comprising M. elsdenii cells, and a growth media; adding an osmolyte protectant molecule; harvesting the M. elsdenii cells; forming a slurry comprising water, a carrier, and the M. elsdenii cells, wherein the carrier has a final concentration of about 1% to about 50% wt and the slurry has a viscosity in the range of about 1 cP to about 500 cP; applying an electrostatic charge to the slurry using a voltage gradient or oscillating voltages; atomizing the slurry to generate a spray of liquid droplets of the slurry; introducing the spray of liquid droplets of the slurry into a drying chamber; and feeding a drying fluid at a temperature between about 50° C. to about 100° C. into the drying chamber to dry the liquid droplets to form the dried powder comprising a plurality of dried particles containing the M. elsdenii cells encapsulated within the carrier, wherein the dried powder comprises less than 15% moisture content, and wherein the entire method is performed under less than 2% oxygen.

In some aspects, provided herein is a method of electrospray drying M. elsdenii cells into a dried powder, comprising: preparing a culture comprising M. elsdenii cells, and a growth media, wherein during the culture the pH, temperature, and/or osmolality are altered for a time period; adding an osmolyte protectant molecule; harvesting the M. elsdenii cells; forming a slurry comprising water, a carrier, and the M. elsdenii cells, wherein the carrier has a final concentration of about 1% to about 50% wt and the slurry has a viscosity in the range of about 1 cP to about 500 cP; applying an electrostatic charge to the slurry using a voltage gradient or oscillating voltages; atomizing the slurry to generate a spray of liquid droplets of the slurry; introducing the spray of liquid droplets of the slurry into a drying chamber; and feeding a drying fluid at a temperature between about 50° C. to about 100° C. into the drying chamber to dry the liquid droplets to form the dried powder comprising a plurality of dried particles containing the M. elsdenii cells encapsulated within the carrier, wherein the dried powder comprises less than 10% moisture content, and wherein the entire method is performed under less than 2% oxygen.

In some aspects, provided herein is a method of electrospray drying M. elsdenii cells into a dried powder, comprising: preparing a culture comprising M. elsdenii cells, and a growth media; harvesting the M. elsdenii cells; adding an osmolyte protectant molecule; forming a slurry comprising water, a carrier, and the M. elsdenii cells, wherein the carrier has a final concentration of about 1% to about 50% wt and the slurry has a viscosity in the range of about 1 cP to about 500 cP; applying an electrostatic charge to the slurry using a voltage gradient or oscillating voltages; atomizing the slurry to generate a spray of liquid droplets of the slurry; introducing the spray of liquid droplets of the slurry into a drying chamber; and feeding a drying fluid at a temperature between about 50°. C to about 100°. C into the drying chamber to dry the liquid droplets to form the dried powder comprising a plurality of dried particles containing the M. elsdenii cells encapsulated within the carrier, wherein the dried powder comprises less than 15% moisture content, and wherein the entire method is performed under less than 2% oxygen.

In some aspects, provided herein is a method of electrospray drying M. elsdenii cells into a dried powder, comprising: preparing a culture comprising M. elsdenii cells, and a growth media, wherein during the culture the pH, temperature, and/or osmolality are altered for a time period; harvesting the M. elsdenii cells; adding an osmolyte protectant molecule; forming a slurry comprising water, a carrier, and the M. elsdenii cells, wherein the carrier has a final concentration of about 1% to about 50% wt and the slurry has a viscosity in the range of about 1 cP to about 500 cP; applying an electrostatic charge to the slurry using a voltage gradient or oscillating voltages; atomizing the slurry to generate a spray of liquid droplets of the slurry; introducing the spray of liquid droplets of the slurry into a drying chamber; and feeding a drying fluid at a temperature between about 50° C. to about 100° C. into the drying chamber to dry the liquid droplets to form the dried powder comprising a plurality of dried particles containing the M. elsdenii cells encapsulated within the carrier, wherein the dried powder comprises less than 10% moisture content, and wherein the entire method is performed under less than 2% oxygen.

In some aspects, provided herein is a method of forming a dry powder comprising M. elsdenii cells encapsulated within a carrier, said method comprising: forming a slurry in a mixing tank comprising water, a carrier, and M. elsdenii cells while stirring the mixing tank with the stirring device; applying an electrostatic charge to the slurry using a voltage gradient or oscillating voltages; atomizing the slurry in an atomizer and discharging atomized slurry into a drying chamber for contact with a drying fluid to form the dried powder containing the M. elsdenii cells encapsulated within the carrier. In some aspects, the drying fluid comprises nitrogen or argon at a temperature between about 50° C. to about 100° C. In some aspects, the dried powder comprises less than 10% moisture content. In some aspects, the entire method is performed under anaerobic conditions. In some aspects, the entire method is performed under less than 2% oxygen.

In some aspects, the drying fluid is selected from the group consisting of nitrogen and argon.

In some aspects, the M. elsdenii cells are concentrated about 20× to about 100× prior to harvesting.

In some aspects, the method further comprises heating the drying fluid to between about 50° C. to about 100° C. by applying a voltage of about 0.1 kV to about 45 kV prior to feeding the drying fluid into the drying chamber.

In some aspects, the method further comprises heating the drying fluid to between about 50° C. to about 100° C. by applying a voltage of about 10 kV to about 30 kV prior to feeding the drying fluid into the drying chamber.

In some aspects, the method further comprises heating the drying fluid to between about 50° C. to about 100° C. by applying a voltage of about 11 kV to about 25 kV prior to feeding the drying fluid into the drying chamber.

In some aspects, the drying fluid is at a temperature between about 60° C. to about 90° C.

In some aspects, the method further comprises heating the drying fluid to between about 60° C. to about 90° C. by applying a voltage of about 10 kV to about 30 kV prior to feeding the drying fluid into the drying chamber.

In some aspects, the voltage is applied constantly or in pulses.

In some aspects, the carrier is selected from the group consisting of a sugar, a sugar alcohol, a sugar derivative, a polysaccharide, an encapsulating polymer, or a nitrogen source, and a mixture thereof. In some aspects, the sugar is sucrose.

In some aspects, the encapsulating polymer is alginate.

In some aspects, the carrier is present in an amount of about 1% to about 40% (w/v).

In some aspects, the dried powder comprises about 1×10³ to about 1×10¹³ CFU/gram of M. elsdenii cells.

In some aspects, the dried powder comprises about 1×10³ CFU/gram of M. elsdenii cells.

In some aspects, the volume of the culture is at least 2-50 liters. In some aspects, the volume of the culture is at least 2 liters.

In some aspects, the volume of the culture is at least 50 liters.

In some aspects, provided herein is a feed additive comprising the electrospray dried M. elsdenii cells produced by any of the methods described herein.

In some aspects, the feed additive further comprises another microorganism.

In some aspects, the feed additive is selected from the group consisting of: a powder, a granulate, a particulate, a pellet, a cake, or combinations thereof.

In some aspects, the feed additive is a probiotic.

In some aspects, provided herein is a composition comprising the electrospray dried M. elsdenii cells produced by any of the methods described herein. In some aspects, the composition comprises any of the feed additives described herein.

In some aspects, the composition is a capsule.

In some aspects, provided herein is a kit comprising the electrospray dried M. elsdenii cells produced by any of the methods described herein, any of the feed additives described herein, or any of the compositions described herein.

In some aspects, provided herein is a method for treating or preventing a condition or disorder associated with lactic acid production in the gastrointestinal tract of an animal, comprising administering to the animal an effective amount of the electrospray dried M. elsdenii cells produced by any of the methods described herein, any of the feed additives described herein, or any of the compositions described herein.

In some aspects, the condition or disorder is acidosis.

In some aspects, wherein the condition or disorder is ruminal acidosis. In some aspects, the condition or disorder is hindgut acidosis.

In some aspects, the condition or disorder is respiratory disease.

In some aspects, the condition or disorder is laminitis.

In some aspects, the condition or disorder is an infection.

In some aspects, the infection is caused by Salmonella or Campylobacter.

In some aspects, provided herein is a method for preventing or decreasing the growth of an opportunistic microorganism in the gastrointestinal tract of an animal, comprising administering to the animal an effective amount of the electrospray dried M elsdenii cells produced by any of the methods described herein, any of the feed additives described herein, or any of the compositions described herein.

In some aspects, the opportunistic microorganism is pathogenic.

In some aspects, the opportunistic microorganism is Salmonella or Campylobacter. In some aspects, the opportunistic microorganism is Escherichia coli.

In some aspects, provided herein is a method of improving the bioavailability of plant-derived phosphorous in the diet of an animal, comprising administering to the animal an effective amount of the electrospray dried M. elsdenii cells produced by any of the methods described herein, any of the feed additives described herein, or any of the compositions described herein.

In some aspects, provided herein is a method of improving growth performance in an animal, comprising administering to the animal an effective amount of the electrospray dried M. elsdenii cells produced by any of the methods described herein, any of the feed additives described herein, or any of the compositions described herein. In some aspects, the improved growth performance in the animal is an improvement in: feed intake, average daily gain, feed conversion ratio, carcass gain, milk production in a milk-producing animal, egg production in poultry, bone mineralization, or combinations thereof.

In some aspects, the electrospray dried M. elsdenii cells, the feed additive, or the composition is administered prior to, concomitantly with, or after feeding the animal with a food.

In some aspects, the method further comprises mixing the electrospray dried M. elsdenii cells or the feed additive with a liquid prior to administration.

In some aspects, the liquid is administered orally or by spraying the animal with the liquid.

In some aspects, the method comprises a single administration of the electrospray dried M. elsdenii cells, feed additive, or composition.

In some aspects, the method comprises a daily administration of the electrospray dried M. elsdenii cells, feed additive, or composition.

In some aspects, the method comprises more than one administration of the electrospray dried M. elsdenii cells, feed additive, or composition on a single day.

In some aspects, the animal is a ruminant.

In some aspects, the ruminant is selected from the group consisting of: cattle, sheep, goats, deer, buffalo, and reindeer.

In some aspects, the animal is a non-ruminant.

In some aspects, the non-ruminant is selected from the group consisting of: equine, poultry animal, and swine.

In some aspects, the poultry animal is selected from the group consisting of: a chicken, a goose, a duck, a quail, a turkey, or a pigeon.

In some aspects, the poultry animal is selected from the group consisting of: a broiler, a broiler breeder, and a layer.

In some aspects, the poultry animal is a chicken.

In some aspects, the equine is a horse, a pony, a donkey, or a mule.

In some aspects, the carrier is selected from the group consisting of a sugar, a sugar alcohol, a sugar derivative, a polysaccharide, an encapsulating polymer, or a nitrogen source, and a mixture thereof. In some aspects, the encapsulating polymer is alginate. In some aspects, the sugar is sucrose.

In some aspects, the dried powder comprises about 1×10³ to about 1×10¹³ CFU/gram of M. elsdenii cells.

In some aspects, the dried powder comprises about 1×10³ CFU/gram of M. elsdenii cells.

In some aspects, provided herein is a dried powder, comprising a plurality of dried particles formed by any of the methods described herein.

In some aspects, provided herein are M. elsdenii cells encapsulated within a carrier, prepared by any of the methods described herein.

In some aspects, provided herein is a system for spray drying anaerobic cells into a dried powder, the system comprising: a source of water, a carrier, and anaerobic cells; a tank comprising a stirring device and the tank is arranged to receive the water, the carrier, and the anaerobic cells from the source to form a slurry having a viscosity in the range of about 1 cP to about 500 cP; an electrode configured to apply an electrostatic charge to the slurry using a voltage gradient or oscillating voltages; an atomizer; a drying chamber comprising an inlet end, an outlet end, and an internal volume located between the inlet end and the outlet end, the internal volume being configured to hold the slurry and a drying fluid. In some aspects, the drying chamber is configured to dry the slurry. In some aspects, the atomizer is configured to: (i) receive the slurry from the tank; and (ii) discharge the slurry into the drying chamber for contact with the drying fluid to form the dried powder containing the anaerobic cells. In some aspects, the drying fluid comprises nitrogen or argon at a temperature between about 50° C. to about 100° C. In some aspects, the entire system is under less than 2% oxygen.

In some aspects, provided herein is a method of electrospray drying anaerobic cells into a dried powder, comprising: preparing a culture comprising anaerobic cells, a growth media, and an osmolyte protectant molecule; harvesting the anaerobic cells; forming a slurry comprising water, a carrier, and the anaerobic cells, wherein the carrier has a final concentration of about 1% to about 50% wt and the slurry has a viscosity in the range of about 1 cP to about 500 cP; applying an electrostatic charge to the slurry using a voltage gradient or oscillating voltages; atomizing the slurry to generate a spray of liquid droplets of the slurry; introducing the spray of liquid droplets of the slurry into a drying chamber; and feeding a drying fluid at a temperature between about 50° C. to about 100° C. into the drying chamber to dry the liquid droplets to form the dried powder comprising a plurality of dried particles containing the anaerobic cells encapsulated within the carrier. In some aspects, the dried powder comprises less than 10% moisture content. In some aspects, the entire method is performed under less than 2% oxygen.

In some aspects, provided herein is a method of electrospray drying anaerobic cells into a dried powder, comprising: preparing a culture comprising anaerobic cells, a growth media, and an osmolyte protectant molecule; harvesting the anaerobic cells; forming a slurry comprising water, a carrier, and the anaerobic cells, wherein the carrier has a final concentration of about 1% to about 50% wt and the slurry has a viscosity in the range of about 1 cP to about 500 cP; applying an electrostatic charge to the slurry using a voltage gradient or oscillating voltages; atomizing the slurry to generate a spray of liquid droplets of the slurry; introducing the spray of liquid droplets of the slurry into a drying chamber; and feeding a drying fluid at a temperature between about 50° C. to about 100° C. into the drying chamber to dry the liquid droplets to form the dried powder comprising a plurality of dried particles containing the anaerobic cells encapsulated within the carrier. In some aspects, during the culture the pH, temperature, and/or osmolality are altered for a time period. In some aspects, the dried powder comprises less than 15% moisture content. In some aspects, the entire method is performed under anaerobic conditions. In some aspects, the entire method is performed under less than 2% oxygen.

In some aspects, the carrier has a final concentration of about 2% to about 30% wt.

In some aspects, provided herein is a method of electrospray drying anaerobic cells into a dried powder, comprising: preparing a culture comprising anaerobic cells, and a growth media; adding an osmolyte protectant molecule; harvesting the anaerobic cells; forming a slurry comprising water, a carrier, and the anaerobic cells, wherein the carrier has a final concentration of about 1% to about 50% wt and the slurry has a viscosity in the range of about 1 cP to about 500 cP; applying an electrostatic charge to the slurry using a voltage gradient or oscillating voltages; atomizing the slurry to generate a spray of liquid droplets of the slurry; introducing the spray of liquid droplets of the slurry into a drying chamber; and feeding a drying fluid at a temperature between about 50° C. to about 100° C. into the drying chamber to dry the liquid droplets to form the dried powder comprising a plurality of dried particles containing the anaerobic cells encapsulated within the carrier, wherein the dried powder comprises less than 15% moisture content, and wherein the entire method is performed under less than 2% oxygen.

In some aspects, provided herein is a method of electrospray drying anaerobic cells into a dried powder, comprising: preparing a culture comprising anaerobic cells, and a growth media, wherein during the culture the pH, temperature, and/or osmolality are altered for a time period; adding an osmolyte protectant molecule; harvesting the anaerobic cells; forming a slurry comprising water, a carrier, and the anaerobic cells, wherein the carrier has a final concentration of about 1% to about 50% wt and the slurry has a viscosity in the range of about 1 cP to about 500 cP; applying an electrostatic charge to the slurry using a voltage gradient or oscillating voltages; atomizing the slurry to generate a spray of liquid droplets of the slurry; introducing the spray of liquid droplets of the slurry into a drying chamber; and feeding a drying fluid at a temperature between about 50° C. to about 100° C. into the drying chamber to dry the liquid droplets to form the dried powder comprising a plurality of dried particles containing the anaerobic cells encapsulated within the carrier, wherein the dried powder comprises less than 10% moisture content, and wherein the entire method is performed under less than 2% oxygen.

In some aspects, the osmolyte protectant molecule is added during growth, before harvest, or after harvest, of the cells.

In some aspects, provided herein is a method of electrospray drying anaerobic cells into a dried powder, comprising: preparing a culture comprising anaerobic cells, and a growth media; harvesting the anaerobic cells; adding an osmolyte protectant molecule; forming a slurry comprising water, a carrier, and the anaerobic cells, wherein the carrier has a final concentration of about 1% to about 50% wt and the slurry has a viscosity in the range of about 1 cP to about 500 cP; applying an electrostatic charge to the slurry using a voltage gradient or oscillating voltages; atomizing the slurry to generate a spray of liquid droplets of the slurry; introducing the spray of liquid droplets of the slurry into a drying chamber; and feeding a drying fluid at a temperature between about 50° C. to about 100° C. into the drying chamber to dry the liquid droplets to form the dried powder comprising a plurality of dried particles containing the anaerobic cells encapsulated within the carrier, wherein the dried powder comprises less than 15% moisture content, and wherein the entire method is performed under less than 2% oxygen.

In some aspects, the osmolyte protectant molecule is added during growth, before harvest, or after harvest, of the cells.

In some aspects, provided herein is a method of electrospray drying anaerobic cells into a dried powder, comprising: preparing a culture comprising anaerobic cells, and a growth media, wherein during the culture the pH, temperature, and/or osmolality are altered for a time period; harvesting the anaerobic cells; adding an osmolyte protectant molecule; forming a slurry comprising water, a carrier, and the anaerobic cells, wherein the carrier has a final concentration of about 1% to about 50% wt and the slurry has a viscosity in the range of about 1 cP to about 500 cP; applying an electrostatic charge to the slurry using a voltage gradient or oscillating voltages; atomizing the slurry to generate a spray of liquid droplets of the slurry; introducing the spray of liquid droplets of the slurry into a drying chamber; and feeding a drying fluid at a temperature between about 50° C. to about 100° C. into the drying chamber to dry the liquid droplets to form the dried powder comprising a plurality of dried particles containing the anaerobic cells encapsulated within the carrier, wherein the dried powder comprises less than 10% moisture content, and wherein the entire method is performed under less than 2% oxygen.

In some aspects, the osmolyte protectant molecule is added during growth, before harvest, or after harvest, of the cells.

In some aspects, provided herein is a method of forming a dry powder comprising anaerobic cells encapsulated within a carrier, said method comprising: forming a slurry in a mixing tank comprising water, a carrier, and anaerobic cells while stirring the mixing tank with the stirring device; applying an electrostatic charge to the slurry using a voltage gradient or oscillating voltages; atomizing the slurry in an atomizer and discharging atomized slurry into a drying chamber for contact with a drying fluid to form the dried powder containing the anaerobic cells encapsulated within the carrier. In some aspects, the drying fluid comprises nitrogen or argon at a temperature between about 50° C. to about 100° C. In some aspects, the dried powder comprises less than 15% moisture content. In some aspects, the entire method is performed under anaerobic conditions. In some aspects, the entire method is under less than 2% oxygen.

In some aspects, the osmolyte protectant molecule is added during growth, before harvest, or after harvest, of the cells.

In some aspects, the drying fluid is selected from the group consisting of nitrogen and argon.

In some aspects, the anaerobic cells are concentrated about 25× to about 100× prior to harvesting.

In some aspects, the method further comprises heating the drying fluid to between about 50° C. to about 100° C. by applying a voltage of about 0.1 kV to about 45 kV prior to feeding the drying fluid into the drying chamber.

In some aspects, the method further comprises heating the drying fluid to between about 50° C. to about 100° C. by applying a voltage of about 10 kV to about 30 kV prior to feeding the drying fluid into the drying chamber.

In some aspects, the method further comprises heating the drying fluid to between about 50° C. to about 100° C. by applying a voltage of about 11 kV to about 25 kV prior to feeding the drying fluid into the drying chamber.

In some aspects, the drying fluid is at a temperature between about 60° C. to about 90° C.

In some aspects, the method further comprises heating the drying fluid to between about 60° C. to about 90° C. by applying a voltage of about 10 kV to about 30 kV prior to feeding the drying fluid into the drying chamber.

In some aspects, the voltage is applied constantly or in pulses.

In some aspects, the carrier is selected from the group consisting of a sucrose, a sugar, a sugar alcohol, a sugar derivative, a polysaccharide, an encapsulating polymer, or a nitrogen source, and a mixture thereof.

In some aspects, the encapsulating polymer is alginate.

In some aspects, the dried powder comprises about 1×10³ to about 1×10¹³ CFU/gram of anaerobic cells.

In some aspects, the dried powder comprises about 1×10³ CFU/gram of anaerobic cells.

In some aspects, provided herein is a dried powder, comprising a plurality of dried particles formed by any of the methods described herein.

In some aspects, provided herein are anaerobic cells encapsulated within a carrier, prepared by any of the methods described herein.

In some aspects, provided herein is a method of improving growth performance in an animal, comprising administering to the animal an effective amount of electrospray dried anaerobic cells produced by any of the methods described herein. In some aspects, the improved growth performance in the animal is an improvement in: feed intake, average daily gain, feed conversion ratio, carcass gain, milk production in a milk-producing animal, egg production in poultry, bone mineralization, or combinations thereof.

In some aspects, provided herein is a method for preventing or decreasing the growth of an opportunistic microorganism in the gastrointestinal tract of an animal, comprising administering to the animal an effective amount of electrospray dried anaerobic cells produced by any of the methods described herein.

In some aspects, provided herein is a composition comprising electrospray dried anaerobic bacterial cells produced by any of the methods described herein.

In some aspects, the M. elsdenii cells are grown at about 30° C. to about 40° C., at about 30° C., at about 31° C., at about 32° C., at about 33° C., at about 34° C., at about 35° C., at about 36° C., at about 37° C., at about 38° C., at about 39° C., or at about 40° C. prior to the harvesting.

In some aspects, the anaerobic cells are grown at about 30° C. to about 40° C., at about 30° C., at about 31° C., at about 32° C., at about 33° C., at about 34° C., at about 35° C., at about 36° C., at about 37° C., at about 38° C., at about 39° C., or at about 40° C. prior to the harvesting.

In some aspects, the slurry is processed at a rate of between about 1 L/hour and about 10,000 L/hour. In some aspects, the slurry is processed at a rate of about 3,000 L/hour.

### BRIEF DESCRIPTION OF THE DRAWINGS/FIGURES

FIG. 1 shows the potential weekly dry powder production from electrospray drying bacteria.

FIG. 2 shows the accelerated shelf life of M. elsdenii ESD and freeze-dried (FD) powder stored at 37° C.

FIG. 3 shows the shelf life of M. elsdenii ESD powder stored at −20° C., 4° C., and 30° C., and FD powder stored at 4° C.

FIG. 4 shows the shelf life of M. elsdenii freeze-dried powder stored at 4° C.

FIG. 5 shows the long-term shelf life of ESD powder processed with an inlet temperature of 80° C., 75° C., or 70° C., and stored at −20° C.

### DETAILED DESCRIPTION OF THE DISCLOSURE

The present disclosure relates to methods for electrospray drying M. elsdenii into a dried powder by applying low amounts of heat. The present disclosure also relates to feed additives and compositions comprising the electrospray dried M. elsdenii cells.

All publications, patents, and other references mentioned herein are incorporated by reference in their entireties for all purposes as if each individual publication or patent application were specifically and individually indicated to be incorporated by reference. In addition, citation or identification of any reference in this application shall not be construed as an admission that such reference is available as prior art to the present invention.

### Terminology

Unless defined otherwise, all technical and scientific terms used herein have the same meaning as commonly understood by one of ordinary skill in the art to which this invention belongs. In case of conflict, the present application including the definitions will control. Unless otherwise required by context, singular terms shall include pluralities and plural terms shall include the singular.

To the extent that section headings are used, they should not be construed as necessarily limiting.

As used herein and in the appended claims, the singular forms “a,” “an,” and “the” include plural references unless the context clearly dictates otherwise. For example, the term “a compound” or “at least one compound” may include a plurality of compounds, including mixtures thereof. The terms “a”, “an,” “the,” “one or more,” and “at least one,” for example, can be used interchangeably herein.

As used herein, the term “about,” when used to modify an amount related to the invention, refers to variation in the numerical quantity that can occur, for example, through routine testing and handling; through inadvertent error in such testing and handling; through differences in the manufacture, source, or purity of ingredients employed in the invention; and the like. Whether or not modified by the term “about”, the claims include equivalents of the recited quantities. In some aspects, the term “about” means plus or minus 10% of the reported numerical value.

Throughout this application, various aspects of this invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range, such as from 1 to 6 should be considered to have specifically disclosed subranges such as from 1 to 2, from 1 to 3, from 1 to 4, from 1 to 5, from 2 to 3, from 2 to 4, from 2 to 5, from 2 to 6, from 3 to 4, from 3 to 5, from 3 to 6, etc., as well as individual numbers within that range, for example, 1, 2, 3, 4, 5, and 6. This applies regardless of the breadth of the range.

The terms “comprises,” “comprising,” “includes,” “including,” “having,” and their conjugates are interchangeable and mean “including but not limited to.” It is understood that wherever aspects are described herein with the language “comprising,” otherwise analogous aspects described in terms of “consisting of” and/or “consisting essentially of” are also provided.

The term “consisting of” means “including and limited to.”

The term “consisting essentially of” means the specified material of a composition, or the specified steps of a method, and those additional materials or steps that do not materially affect the basic characteristics of the material or method.

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

The terms “culture,” “to culture,” and “culturing,” as used herein, means to incubate cells under in vitro conditions that allow for cell growth or division or to maintain cells in a living state. The term “a culture” can also be used herein to refer to cells incubated under in vitro conditions (e.g., cells incubated a liquid growth media).

The term “probiotic” as used herein refers to one or more live microorganisms (bacteria and/or yeast) and may or may not include other ingredients, which when administered in adequate amounts may confer a health benefit on the animal or subject.

The term “direct-fed microbial product” as used in herein refers to a product that contains one or more live microorganisms (bacteria and/or yeast) and may or may not include other ingredients, that can be administered to the animal or subject in feed mixtures, boluses, and/or oral pastes, and when administered in adequate amounts may confer a health benefit on the animal or subject.

The term “feed additive” as used herein refers to one or more ingredients, products, or substances (e.g., cells), used alone or together, in nutrition (e.g., to improve the quality of a food (e.g., an animal feed), to improve an animal's performance and health, and/or to enhance digestibility of a food or materials within a food). A feed additive can be, for example, a probiotic.

The terms “growth media” and “culture media” as used herein refer to a solid (e.g., agar), semi-solid (e.g., agar), or liquid (e.g., broth) composition that contains components to support the growth of cells.

The terms “harvest” and “harvesting” as used herein refer to collecting cells from a culture, e.g., collecting cells in growth media from the culture, collecting cells by removing an amount of the growth media from the cells (e.g., by concentrating the cells in a liquid culture or separating the cells from the growth media), or halting the culturing of the cells. The terms include collecting or removing a volume of liquid comprising the cells from a liquid culture, including a volume in which the cells have been concentrated.

The term “isolated” as used herein does not necessarily reflect the extent to which an isolate has been purified but indicates isolation or separation from a native form or native environment. An isolate can include, but is not limited to, an isolated microorganism, an isolated biomass, or an isolated culture.

As used herein, “excipient” refers to a component, or mixture of components, that is used to give desirable characteristics to a feed additive, food, composition, or pharmaceutical composition as disclosed herein. An excipient of the present invention can be described as a “pharmaceutically acceptable” excipient when added to a pharmaceutical composition, meaning that the excipient is a compound, material, composition, salt, and/or dosage form which is, within the scope of sound medical judgment, suitable for contact with tissues of animals (i.e., human and non-human animals) without excessive toxicity, irritation, allergic response, or other problematic complications over the desired duration of contact commensurate with a reasonable benefit/risk ratio.

As used herein, the term “yield” refers to the amount of living, or viable, cells, including the amount in a particular volume (e.g., colony-forming units per milliliter (“CFU/mL”)) or in a particular weight (e.g., CFU per gram (“CFU/g”)).

As used herein, the term “viable” refers to a living organism or organisms (e.g., a microbial cell that is alive or microbial cells that are alive). “Viability” refers to the ability to live, especially under certain conditions.

As used herein, “purify,” “purified,” and “purification” mean to make substantially pure or clear from unwanted components, material defilement, admixture or imperfection.

The terms “animal” or “subject” refer to any organism belonging to the kingdom Animalia and includes without limitation, unless otherwise noted, aquatic animals and terrestrial animals such as fish; commercial fish; ornamental fish; fish larvae; bivalves; mollusks; crustaceans; shellfish; shrimp; larval shrimp; artemia; rotifers; brine shrimp; filter feeders; amphibians; reptiles; mammals; non-human animals; domestic animals; farm animals; zoo animals; sport animals; breeding stock; racing animals; show animals; heirloom animals; rare or endangered animals; companion animals; pet animals such as dogs, cats, guinea pigs, rabbits, rats, mice, or horses; primates such as monkeys (e.g., cebus, rhesus, African green, patas, cynomolgus, and cercopithecus), apes, orangutans, baboons, gibbons, and chimpanzees; canids such as dogs and wolves; felids such as cats, lions, and tigers; equids such as horses, ponies, donkeys, mules, and zebras; food animals such as cows, buffalo, cattle, pigs, poultry, and sheep; ungulates such as deer and giraffes; avians (i.e., birds); poultry such as chickens, geese, ducks, quails, turkeys, pigeons, emus, ostriches, and any other bird used as a food or farm animal, including broilers, broiler breeders, and a layers; rodents such as mice, rats, hamsters and guinea pigs; and so on. In some aspects, the subject is a mammal. In some aspects, the mammal is a human subject. In some aspects, the mammal excludes a human subject. An animal feed includes, but is not limited to, an aquaculture feed, a domestic animal feed including a pet feed, a zoological animal feed, a work animal feed, a livestock feed, and combinations thereof. In some aspects, food includes animal feed and human food.

It is appreciated that certain features of the invention, which are, for clarity, described in the context of separate aspects, can also be provided in combination in a single aspect. Conversely, various features of the invention, which are, for brevity, described in the context of a single aspect, can also be provided separately or in any suitable subcombination or as suitable in any other described aspect of the invention. Certain features described in the context of various aspects are not to be considered essential features of those aspects unless the aspect is inoperative without those elements.

Although methods and materials similar or equivalent to those described herein can be used in practice or testing of the present invention, suitable methods and materials are described below. The materials, methods and examples are illustrative only and are not intended to be limiting. Other features and advantages of the invention will be apparent from the detailed description and from the claims.

M. elsdenii

M. elsdenii cells from any strain or any combination of strains can be used in the disclosure described herein.

A M. elsdenii strain or strains can be selected from a stock culture collection (e.g., American Type Culture Collection (“ATCC®”), National Collection of Industrial, Food and Marine Bacteria (“NCIMB”), National Collection of Type Cultures (“NCTC”), American Research Service (“ARC”) culture collection (i.e., “NRRL”), National Institute of Animal Health (NIAH) culture collection, or can be a strain that has been isolated from a natural source (e.g., from the gastrointestinal tract of a ruminant).

Examples of M. elsdenii strains that can be selected from a culture collection include, but are not limited to, the strains listed by deposit numbers in Table 1. Alternative designations of the deposit numbers are also indicated.

TABLE 1
Examples of M. elsdenii Strains and Source of Each Strain.

| Deposit Number | Alternative Designations | Source of Strain |
|---|---|---|
| ATCC ® 25940 | NCIMB 8927; BE2-2083 | Rumen of Sheep |
| ATCC ® 17752 | B159; NCIMB 702409; NCDO2409 | N/A |
| ATCC ® 17753 | T81; NCIMB 702410; NCDO2410 | N/A |
| NCIMB 702261 | A17-2; A12-2; NCDO2261 | Human Adult Faeces |
| NCIMB 702262 | S17-3; NCDO2262 | Juvenile Swine Faeces |
| NCIMB 702264 | LC1 | N/A |
| NCIMB 702331 | LC1; NCDO2263; NCDO2264; | N/A |
| | NCDO2331 | |
| NCIMB 41125 | | Rumen of Dairy Cow |
| NCIMB 41787 | | Rumen of Dairy Cow |
| NCIMB 41788 | | Rumen of Dairy Cow |
| NRRL 18624 | | Rumen of Bovine |
| NIAH 1102 | | N/A |
| CBS146325 (NRRL) | | |
| CBS146326 (NRRL) | | |
| CBS146327 (NRRL) | | |
| CBS146328 (NRRL) | | |
| CBS146329 (NRRL) | | |
| CBS146330 (NRRL) | | |

In some aspects, the M. elsdenii cells are from a strain having a deposit number selected from the group consisting of: ATCC® 25940, ATCC® 17752, ATCC® 17753, NCIMB 702261, NCIMB 702262, NCIMB 702264, NCIMB 702331, NCIMB 702409, NCIMB 702410, NCIMB 41125, NCIMB 41787, NCIMB 41788, NRRL 18624, NIAH 1102, CBS146325, CBS146326, CBS146327, CBS146328, CBS146329, CBS146330, and combinations thereof, including any of the alternative designations in Table 1 or any commercially available sources of M. elsdenii.

In some aspects, the M. elsdenii cells are from a strain isolated from a ruminant (e.g., a cow). See, e.g., U.S. Pat. No. 7,550,139.

In some aspects, the M. elsdenii cells are from a strain isolated from a non-ruminant (e.g., a human).

In some aspects, the M. elsdenii cells are from a strain selected for lactate utilization (e.g., a strain that utilizes lactate in the presence of sugars), resistance to ionophore antibiotics, relatively high growth rate, capability to produce predominantly acetate, capability to proliferate at pH values below 5.0 and as low as 4.5, production of volatile fatty acids (VFAs), phytase activity, and combinations thereof. See, e.g., U.S. Pat. No. 7,550,139.

In some aspects, a strain selected for lactate utilization utilizes lactate as a preferred carbon source in the presence of a soluble carbohydrate (e.g., glucose and/or maltose). Lactate utilization can be determined, for example, based on growth in a medium containing lactate and lacking soluble carbohydrates as compared to the same medium supplemented with soluble carbohydrates.

In some aspects, the M. elsdenii cells are from a strain with a high growth rate as compared to other strains. The growth rates of different strains can be determined, for example, by culturing the cells in a liquid medium and monitoring the increase in optical density over time.

In some aspects, the M. elsdenii cells are from a strain with phytase activity.

In some aspects, the M. elsdenii cells are from M. elsdenii strain NCIMB 41125. This strain of M. elsdenii has a high specific growth rate (0.94 generations/hour), is capable of growth in a pH range of 4.5 to 6.5 or more, uses D- and L-Lactate as its preferred substrate, but also has the ability to utilize glucose and other carbohydrates, and tolerates ionophores.

In some aspects, the M. elsdenii cells are from M. elsdenii strain NCIMB 41787. In some aspects, the M. elsdenii cells are from M. elsdenii strain NCIMB 41788.

In some aspects, the M. elsdenii cells are from M. elsdenii strain ATCC® 25940.

In some aspects, the M. elsdenii cells are derived from a strain selected from a stock culture collection or isolated from a natural source. Cells that are “derived” from a strain can be a natural or artificial derivative such as, for example, a sub isolate, a mutant, variant, or recombinant strain.

In some aspects, the M. elsdenii are freeze-dried. See WO 2018/144653 A1, which is herein incorporated by reference in its entirety.

Preparing a Culture Comprising Anaerobic Bacterial Cells or M. elsdenii Cells

Anaerobic bacteria, including M. elsdenii should be cultured under anaerobic conditions in order to obtain maximum yield and viability. In some aspects, anaerobic bacteria (e.g., M. elsdenii) should be cultured under less than 2% oxygen.

In some aspects, a culture comprises M. elsdenii cells and a growth media.

In some aspects, the culture comprises one or more strains of M. elsdenii cells. In some aspects, the culture comprises a single strain of M. elsdenii cells. In some aspects, the culture consists of one or more strains of M. elsdenii cells (i.e., the cells in the culture consist of M. elsdenii cells, e.g., one or more strains of M. elsdenii cells). In some aspects, the culture consists of a single strain of M. elsdenii cells.

In some aspects, a culture comprises one or more strains of anaerobic bacterial cells and a growth media. In some aspects, the culture comprises Bifidobacterium cells, such as B. breve, Lactobacillus cells, such as L. plantarum, Bifidobacterium cells, such as B. animalis subsp. lactis, Pediococcus cells, such as P. acidilactici, Lactobacillus cells, such as L. casei, Fibrobacter, such as F. succinogenes, and Butyrivibrio, such as B. fibrisolvens, Ruminococcus, such as R. flavefaciens, and a growth media.

Various fermentation parameters for inoculating, growing, and harvesting anaerobic cells (e.g., M. elsdenii cells) can be used, including continuous fermentation (i.e., continuous culture) or batch fermentation (i.e., batch culture). See, for example, U.S. Pat. No. 7,550,139.

Growth media for anaerobic cells (e.g., M. elsdenii cells) can be a solid, semi-solid, or liquid. A medium can contain nutrients that provide essential elements and specific factors that enable growth. A variety of microbiological media and variations are well known in the art. Media can be added to a culture at any time, including the start of the culture, during the culture, or intermittently/continuously.

Examples of growth media include, but are not limited to: (1) semi-defined media, which contains peptone, 3 g/L; yeast, 3 g/L; vitamin solution, 2 mL/L; mineral solution, 25 mL/L; indigo carmine (0.5%), 1 g/L; 12.5% L-cysteine, 2 g/L; 12.5% sodium sulfide, 2 g/L; and supplemented with either Na-lactate (semi-defined lactate, SDL), glucose (semi-defined glucose, SDG), or maltose (semi-defined maltose, SDM); (2) Modified Reinforced Clostridial Agar/Broth Medium (pre-reduced), which contains peptone, 10 g/L; beef extract, 10 g/L; yeast extract, 3 g/L; dextrose 5 g/L; NaCl, 5 g/L; soluble starch, 1 g/L; L-cysteine HCl, 0.5 g/L; sodium acetate, 3 g/L; and resazurin (0.025%), 4 mL/L; (3) Trypticase soy agar/broth with defibrinated sheep blood; (4) semi-defined rumen fluid free medium, which contains Na-lactate (70%), 10 g/L; Peptone, 2 g/L; KH₂PO₄ 1 g/L; (NH₄)₂SO₄ 3 g/L; MgSO₄7H₂O 0.2 g/L; CaCl₂·2H₂O 0.06 g/L; Vitamins (Pyridoxolhydrochloride, 4 mg/L; Pyridoxamine, 4 mg/L; Riboflavin, 4 mg/L; Thiaminiumchloride, 4 mg/L; Nicotinamide, 4 mg/L; Ca-D-pantothenate, 4 mg/L; 4-Aminobenzoic acid, 0.2 mg/L, Biotin, 0.2 mg/L, Folic acid, 0.1 mg/L and Cyanocobalamin, 0.02 mg/L); Na₂S·9H₂O, 0.25 g/L; Cysteine, 0.25 g/L; Antifoam, 0.07 mL/L and Monensin, 10 mg/L; and which is prepared by adding the Na-lactate and mineral solution to a reservoir bottle and autoclaving for 60 minutes; dissolving the peptone in 300 mL distilled H₂O and autoclaving separately; filter sterilizing the vitamin solution and two reducing agents beforehand; following autoclaving, gassing the reservoir bottle with anaerobic gas overnight; adding the other constituents separately after cooling; and adjusting the pH to the desired value with 5N HCl; and (5) incubated rumen fluid lactate (“IRFL”) medium, which contains 400 mL incubated clarified rumen fluid from lucerne-fed sheep, 371 mL distilled water, 2 g peptone, 15 g agar, 100 mL 10% (w/v) sodium-D, L-lactate solution, 100 mL 0.04% (w/v) bromocresol purple solution, and 25 mL mineral solution containing 40 g/L KH₂PO₄; 120 g/L (NH₄)₂SO₄; 8 g/L MgSO₄·7H₂O and 2.4 g/L CaCl₂)·2H₂O, where lactic acid (90% w/v) is used to adjust the pH to 5.5 before autoclaving at 121° C. for 25 minutes, then cooling in a 50° C. water bath while being gassed with an anaerobic gas mixture, followed by adding two milliliters of each of Na₂S·9H₂O (12.5% w/v) and cysteine·HCl·H₂O (12.5% w/v).

In some aspects, the culture comprises a growth media comprising at least two carbon sources. In some aspects, the at least two carbon sources are selected from the group consisting of: casein, starch (e.g., gelatinized starch and/or soluble starch), lactate (i.e., lactic acid), dextrose, fructose, fructan, glucose, sucrose, lactose, maltose, acetate, glycerol, mannitol, sorbitol, saccharose, xylose, molasses, fucose, glucosamine, dextran, a fat, an oil, glycerol, sodium acetate, arabinose, soy protein, soluble protein, raffinose, amylose, starch, tryptone, yeast extract and combinations thereof.

In some aspects, the at least two carbon sources consist of about 1-99% of a first carbon source (e.g., any carbon source described herein) and about 1-99% of a second carbon source (e.g., any carbon source described herein that is different from the first carbon source), wherein 100% of the at least two carbon sources consist of the first carbon source and the second carbon source. In some aspects, the at least two carbon sources consist of about 50-60% of the first carbon source and about 40-50% of the second carbon source, about 50-70% of the first carbon source and about 30-50% of the second carbon source, about 50-80% of the first carbon source and about 20-50% of the second carbon source, or about 50-90% of the first carbon source and about 10-50% of the second carbon source. In other aspects, the at least two carbon sources consist of about 65-75% of the first carbon source and about 25-35% of the second carbon source. In some aspects, the first carbon source is lactate.

In some aspects, the culture may further comprise an osmolyte protectant molecule. In some aspects, the osmolyte protectant molecule is added before harvesting the cells. In some aspects, the osmolyte protectant molecule is added after harvesting the cells. An osmolyte protectant molecule can protect the anaerobic cells (e.g., M. elsdenii cells) from the stresses caused by electrospray drying. This results in a longer shelf life and higher CFU/g after electrospray drying. Examples of classes of an osmolyte protectant molecule, but not limited to, are amino acids, sugars, polyols, methylamines, and methyl sulfonium compounds. In some aspects, these classes are added to the culture between about 0.001 molar (M) to 0.5 M. In some aspects, the osmolyte protectant molecule is a methylamine. In some aspects, the methylamine is betaine. In some aspects, the osmolyte protectant molecule is betaine. In some aspects, the betaine is added to the culture between about 0.001 molar (M) to 0.5 M. In some aspects, the osmolyte protectant molecule is urea. In some aspects, the urea is added to the culture between about 0.001 molar (M) to 0.5 M. In some aspects, the osmolyte protectant molecule is trimethylamine N-oxide (TMAO). In some aspects, the trimethylamine N-oxide (TMAO) is added to the culture between about 0.001 molar (M) to 0.5 M. In some aspects, the osmolyte protectant molecule is dimethylsulfoniopropionate. In some aspects, the dimethylsulfoniopropionate is added to the culture between about 0.001 molar (M) to 0.5 M. In some aspects, the osmolyte protectant molecule is sarcosine. In some aspects, the sarcosine is added to the culture between about 0.001 molar (M) to 0.5 M. In some aspects, the osmolyte protectant molecule is glycerophosphorylcholine. In some aspects, the glycerophosphorylcholine is added to the culture between about 0.001 molar (M) to 0.5 M. In some aspects, the osmolyte protectant molecule is myo-inositol. In some aspects, the myo-inositol is added to the culture between about 0.001 molar (M) to 0.5 M. In some aspects, the osmolyte protectant molecule is taurine. In some aspects, the taurine is added to the culture between about 0.001 molar (M) to 0.5 M. In some aspects, the osmolyte protectant molecule is glycine. In some aspects, the glycine is added to the culture between about 0.001 molar (M) to 0.5 M.

In some aspects, the anaerobic cells (e.g., M. elsdenii cells) are grown at about 39° C. to about 40° C., at about 35° C., at about 36° C., at about 37° C., at about 38° C., at about 39° C., or at about 40° C.

In some aspects, the temperature of the culture comprising the anaerobic cells (e.g., M. elsdenii cells) may be increased for a time period to induce the production of stress-resistance molecules in the cells (e.g., heat shock protein and/or cold shock proteins), and then returning the temperature to the original setpoint. This results in electrospray dried particles that have a longer shelf life and higher CFU/g after electrospray drying. In some aspects, the temperature is increased from the culture process setpoint by 1-30° C. In some aspects, the temperature is increased from the culture process setpoint by 2-29° C. In some aspects, the temperature is increased from the culture process setpoint by 3-28° C. In some aspects, the temperature is increased from the culture process setpoint by 4-27° C. In some aspects, the temperature is increased from the culture process setpoint by 5-26° C. In some aspects, the temperature is increased from the culture process setpoint by 6-25° C. In some aspects, the temperature is increased from the culture process setpoint by 7-24° C. In some aspects, the temperature is increased from the culture process setpoint by 8-23° C. In some aspects, the temperature is increased from the culture process setpoint by 9-22° C. In some aspects, the temperature is increased from the culture process setpoint by 10-21° C. In some aspects, the temperature is increased from the culture process setpoint by 11-20° C. In some aspects, the temperature is increased from the culture process setpoint by 12-19° C. In some aspects, the temperature is increased from the culture process setpoint by 13-18° C. In some aspects, the temperature is increased from the culture process setpoint by 14-17° C. In some aspects, the temperature is increased from the culture process setpoint by 15-16° C. In some aspects, the temperature is increased from the culture process setpoint by 1° C., 2° C., 3° C., 4° C., or 5° C., or 1-5° C.

In some aspects, the time period of increasing the temperature of the culture is from about 1 minute to 12 hours. In some aspects, the time period of increasing the temperature of the culture is from about 30 minutes to 11 hours. In some aspects, the time period of increasing the temperature of the culture is from about 1-10 hours. In some aspects, the time period of increasing the temperature of the culture is from about 2-9 hours. In some aspects, the time period of increasing the temperature of the culture is from about 3-8 hours. In some aspects, the time period of increasing the temperature of the culture is from about 4-7 hours. In some aspects, the time period of increasing the temperature of the culture is from about 5-6 hours.

In some aspects, the anaerobic cells (e.g., M. elsdenii cells) are cooled to about 18° C. to about 25° C. for storage.

In some aspects, the pH of the culture comprising the anaerobic cells, e.g., M. elsdenii cells, (e.g., during the culturing and/or at the time of harvesting) is between about 4.0 to about 8.0, between about 4.0 to about 7.5, between about 4.0 to about 7.0, between about 4.0 to about 6.5, between about 4.0 to about 6.0, between about 4.0 to about 5.5, between about 4.0 to about 5.0, between about 4.0 to about 4.5, between about 4.5 to about 8.0, between about 4.5 to about 7.5 between about 4.5 to about 7.0, between about 4.5 to about 6.5, between about 4.5 to about 6.0, between about 4.5 to about 5.5, between about 4.5 to about 5.0, between about 4.6 to about 6.9, between about 4.7 to about 6.8, between about 4.8 to about 6.7, between about 4.9 to about 6.6, between about 5.0 to about 7.0, between about 5.0 to about 6.5, between about 5.0 to about 6.0, between about 5.0 to about 5.5, between about 5.1 to about 6.9, between about 5.2 to about 6.8, between about 5.3 to about 6.7, between about 5.4 to about 6.6, between about 5.5 to about 7.0, between about 5.5 to about 6.5, between about 5.1 to about 6.4, between about 5.2 to about 6.3, between about 5.3 to about 6.2, between about 5.4 to about 6.1, between about 5.5 to about 6.0, between about 5.0 to about 6.1, between about 5.0 to about 6.2, between about 5.0 to about 6.3, between about 5.0 to about 6.4, between about 5.1 to about 6.5, between about 5.2 to about 6.5, between about 5.3 to about 6.5, or between about 5.4 to about 6.5.

In some aspects, the pH of the culture comprising the anaerobic cells (e.g., M. elsdenii cells) may be reduced for a time period to induce the production of stress-resistance molecules in the cells (e.g., heat shock protein and/or cold shock proteins), and then returning the pH to the original setpoint. This results in electrospray dried particles that have a longer shelf life and higher CFU/g after electrospray drying. In some aspects, the pH is reduced from the culture process setpoint by 0.1-6.0 pH units. In some aspects, the pH is reduced from the culture process setpoint by 0.5-5.5 pH units. In some aspects, the pH is reduced from the culture process setpoint by 1.0-5.0 pH units. In some aspects, the pH is reduced from the culture process setpoint by 1.5-4.5 pH units. In some aspects, the pH is reduced from the culture process setpoint by 2.0-4.0 pH units.

In some aspects, the pH of the culture comprising the anaerobic cells (e.g., M. elsdenii cells) may be increased for a time period to induce the production of stress-resistance molecules in the cells (e.g., heat shock protein and/or cold shock proteins), and then returning the pH to the original setpoint. This results in electrospray dried particles that have a longer shelf life and higher CFU/g after electrospray drying. In some aspects, the pH is increased from the culture process setpoint by 0.1-6.0 pH units. In some aspects, the pH is increased from the culture process setpoint by 0.5-5.5 pH units. In some aspects, the pH is increased from the culture process setpoint by 1.0-5.0 pH units. In some aspects, the pH is increased from the culture process setpoint by 1.5-4.5 pH units. In some aspects, the pH is increased from the culture process setpoint by 2.0-4.0 pH units.

In some aspects, the time period of reducing or increasing the pH of the culture is from about 1 minute to 12 hours. In some aspects, the time period of reducing or increasing the pH of the culture is from about 30 minutes to 11 hours. In some aspects, the time period of reducing or increasing the pH of the culture is from about 1-10 hours. In some aspects, the time period of reducing or increasing the pH of the culture is from about 2-9 hours. In some aspects, the time period of reducing or increasing the pH of the culture is from 3-8 hours. In some aspects, the time period of reducing or increasing the pH of the culture is from 4-7 hours. In some aspects, the time period of reducing or increasing the pH of the culture is from 5-6 hours.

In some aspects, the osmolality of the culture comprising the anaerobic cells (e.g., M. elsdenii cells) may be increased for a time period to induce the production of stress-resistance molecules in the cells (e.g., heat shock protein and/or cold shock proteins), and then returning the osmolality to the original setpoint. This results in electrospray dried particles that have a longer shelf life and higher CFU/g after electrospray drying. In some aspects, the osmolality of the culture can be increased by adding salt to the culture between about 0.2 M-2 M. In some aspects, the osmolality of the culture can be increased by adding salt to the culture between about 0.3 M-1.9 M. In some aspects, the osmolality of the culture can be increased by adding salt to the culture between about 0.4 M-1.8 M. In some aspects, the osmolality of the culture can be increased by adding salt to the culture between about 0.5 M-1.7 M. In some aspects, the osmolality of the culture can be increased by adding salt to the culture between about 0.6 M-1.6 M. In some aspects, the osmolality of the culture can be increased by adding salt to the culture between about 0.7 M-1.5 M. In some aspects, the osmolality of the culture can be increased by adding salt to the culture between about 0.8 M-1.4 M. In some aspects, the osmolality of the culture can be increased by adding salt to the culture between about 0.9 M-1.3 M. In some aspects, the osmolality of the culture can be increased by adding salt to the culture between about 1.0 M-1.2 M.

In some aspects, the time period of increasing the osmolality of the culture is from about 1 minute to 12 hours. In some aspects, the time period of increasing the osmolality of the culture is from about 30 minutes to 1 hours. In some aspects, the time period of increasing the osmolality of the culture is from about 1-10 hours. In some aspects, the time period of increasing the osmolality of the culture is from 2-9 hours. In some aspects, the time period of increasing the osmolality of the culture is from 3-8 hours. In some aspects, the time period of increasing the osmolality of the culture is from 4-7 hours. In some aspects, the time period of increasing the osmolality of the culture is from 5-6 hours.

In some aspects, the osmolality of the culture comprising the anaerobic cells (e.g., M. elsdenii cells) may be decreased for a time period to induce the production of stress-resistance molecules in the cells (e.g., heat shock protein and/or cold shock proteins), and then returning the osmolality to the original setpoint.

To culture anaerobic cells (e.g., M. elsdenii cells), fermenters of different sizes and designs that maintain anaerobic conditions can be used. In some aspects, culturing anaerobic cells should be performed under less than 2% oxygen. A fermenter can be capable, for example, of fermenting culture volumes sufficient for commercial production of the anaerobic cells (e.g., M. elsdenii cells).

In some aspects, the culture volume is about 2 liters, about 5 liters, about 10 liters, about 50 liters, about 100 liters, about 150 liters, about 200 liters, about 250 liters, about 300 liters, about 350 liters, about 400 liters, about 450 liters, about 500 liters, about 600 liters, about 800 liters, about 1,000 liters, about 1,200 liters, about 1,500 liters, about 1,800 liters, about 2,000 liters, about 2, 200 liters, about 2,500 liters, about 2,750 liters, about 3,000 liters, about 4,000 liters, about 5,000 liters, about 6,000 liters, about 7,000 liters, about 8,000 liters, about 9,000 liters, about 10,000 liters, about 20,000 liters, about 50,000 liters, or about 75,000 liters.

In some aspects, the culture volume is about 2 liters to about 75,000 liters, about 2 liters to about 70,000 liters, about 2 liters to about 65,000 liters, about 2 liters to about 60,000 liters, about 2 liters to about 55,000 liters, about 2 liters to about 50,000 liters, about 2 liters to about 45,000 liters, about 2 liters to about 40,000 liters, about 2 liters to about 35,000 liters, about 2 liters to about 30,000 liters, about 2 liters to about 25,000 liters, about 2 liters to about 20,000 liters, about 2 liters to about 15,000 liters, about 2 liters to about 10,000 liters, about 2 liters to about 5,000 liters, about 2 liters to about 2,500 liters, about 2 liters to about 500 liters, about 2 liters to about 250 liters, about 2 liters to about 100 liters, about 2 liters to about 50 liters, about 2 liters to about 25 liters, or about 2 liters to about 10 liters.

In some aspects, the culture volume is about 5 liters to about 75,000 liters, about 5 liters to about 70,000 liters, about 5 liters to about 65,000 liters, about 5 liters to about 60,000 liters, about 5 liters to about 55,000 liters, about 5 liters to about 50,000 liters, about 5 liters to about 45,000 liters, about 5 liters to about 40,000 liters, about 5 liters to about 35,000 liters, about 5 liters to about 30,000 liters, about 5 liters to about 25,000 liters, about 5 liters to about 20,000 liters, about 5 liters to about 15,000 liters, about 5 liters to about 10,000 liters, about 5 liters to about 5,000 liters, about 5 liters to about 2,500 liters, about 5 liters to about 500 liters, about 5 liters to about 250 liters, about 5 liters to about 100 liters, about 5 liters to about 50 liters, about 5 liters to about 25 liters, or about 5 liters to about 10 liters.

In some aspects, the culture volume is about 10 liters to about 75,000 liters, about 10 liters to about 70,000 liters, about 10 liters to about 65,000 liters, about 10 liters to about 60,000 liters, about 10 liters to about 55,000 liters, about 10 liters to about 50,000 liters, about 10 liters to about 45,000 liters, about 10 liters to about 40,000 liters, about 10 liters to about 35,000 liters, about 10 liters to about 30,000 liters, about 10 liters to about 25,000 liters, about 10 liters to about 20,000 liters, about 10 liters to about 15,000 liters, about 10 liters to about 10,000 liters, about 10 liters to about 5,000 liters, about 10 liters to about 2,500 liters, about 10 liters to about 500 liters, about 10 liters to about 250 liters, about 10 liters to about 100 liters, about 10 liters to about 50 liters, or about 10 liters to about 25 liters.

In some aspects, the culture volume is about 250 liters to about 750 liters, about 300 liters to about 800 liters, about 350 liters to about 850 liters, about 400 liters to about 900 liters, about 450 liters to about 950 liters, about 500 liters to about 1,000 liters, about 750 liters to about 1,250 liters, about 1,000 liters to about 2,000 liters, about 2,000 liters to about 4,000 liters, about 4,000 liters to about 8,000 liters, about 5,000 liters to about 10,000 liters, about 50 liters to about 75,000 liters, about 50 liters to about 50,000 liters, about 50 liters to about 25,000 liters, about 50 liters to about 20,000 liters, about 50 liters to about 15,000 liters, about 50 liters to about 10,000 liters, about 100 liters to about 10,000 liters, about 100 liters to about 5,000 liters, about 100 liters to about 4,000 liters, about 100 liters to about 3,000 liters, about 100 liters to about 2,900 liters, about 100 liters to about 2,850 liters, about 100 liters to about 2,800 liters, about 100 liters to about 2,750 liters.

In some aspects, the culture comprises a liquid, and the method comprises harvesting the anaerobic cells (e.g., M. elsdenii cells) by removing a percentage of the liquid. In some aspects, harvesting the cells comprises removing about 5% to about 100%, about 10% to about 100%, about 15% to about 100%, about 20% to about 100%, about 25% to about 100%, about 30% to about 100%, about 35% to about 100%, about 40% to about 100%, about 45% to about 100%, about 50% to about 100% of the liquid, about 55% to about 100%, about 60% to about 100%, about 65% to about 100%, about 70% to about 100%, about 75% to about 100%, about 80% to about 100%, about 85% to about 100%, about 90% to about 100%, or about 95% to about 100% of the liquid. In some aspects, harvesting the cells comprises removing at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or at least 100% of the liquid. In some aspects, the cells are concentrated about 1-100×. In some aspects, the cells are concentrated about 5-100×. In some aspects, the cells are concentrated about 10-100×. In some aspects, the cells are concentrated about 15-100×. In some aspects, the cells are concentrated about 20-100×. In some aspects, the cells are concentrated about 25-100×. In some aspects, the cells are concentrated about 30-100×. In some aspects, the cells are concentrated about 35-100×. In some aspects, the cells are concentrated about 40-100×.

In some aspects, the culture comprises a liquid, and the method comprises concentrating the anaerobic cells (e.g., M. elsdenii cells) by removing a percentage of the liquid prior to harvesting. In some aspects, concentrating the cells comprises removing about 5% to about 100%, about 10% to about 100%, about 15% to about 100%, about 20% to about 100%, about 25% to about 100%, about 30% to about 100%, about 35% to about 100%, about 40% to about 100%, about 45% to about 100%, about 50% to about 100% of the liquid, about 55% to about 100%, about 60% to about 100%, about 65% to about 100%, about 70% to about 100%, about 75% to about 100%, about 80% to about 100%, about 85% to about 100%, about 90% to about 100%, or about 95% to about 100% of the liquid. In some aspects, concentrating the cells comprises removing at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or at least 100% of the liquid. In some aspects, the cells are concentrated about 1-100×. In some aspects, the cells are concentrated about 5-100×. In some aspects, the cells are concentrated about 10-100×. In some aspects, the cells are concentrated about 15-100×. In some aspects some aspects, the cells are concentrated about 20-100×. In some aspects, the cells are concentrated about 25-100×. In some aspects, the cells are concentrated about 30-100×. In some aspects, the cells are concentrated about 35-100×. In some aspects, the cells are concentrated about 40-100×.

In some aspects, the method comprises harvesting the anaerobic cells (e.g., M. elsdenii cells) by concentrating the cells. In some aspects, harvesting the cells comprises concentrating the cells by at least one technique selected from the group consisting of: centrifugation, filtration, dialysis, reverse osmosis, and combinations thereof. In some aspects, the filtration comprises clay filtration. In some aspects, the filtration comprises flocculation with clay. In some aspects, the filtration comprises flocculation with a combination of clay and chitosan. In some aspects, the filtration comprises tangential flow filtration, also known as cross-flow filtration.

In some aspects, the pH of the culture comprising the anaerobic cells (e.g., M. elsdenii cells) at the time of harvesting is between about 4.5 to about 7.0, between about 4.5 to about 6.5, between about 4.5 to about 6.0, between about 4.5 to about 5.5, between about 4.5 to about 5.0, between about 4.6 to about 6.9, between about 4.7 to about 6.8, between about 4.8 to about 6.7, between about 4.9 to about 6.6, between about 5.0 to about 7.0, between about 5.0 to about 6.5, between about 5.0 to about 6.0, between about 5.0 to about 5.5, between about 5.1 to about 6.9, between about 5.2 to about 6.8, between about 5.3 to about 6.7, between about 5.4 to about 6.6, between about 5.5 to about 7.0, between about 5.5 to about 6.5, between about 5.1 to about 6.4, between about 5.2 to about 6.3, between about 5.3 to about 6.2, between about 5.4 to about 6.1, between about 5.5 to about 6.0, between about 5.0 to about 6.1, between about 5.0 to about 6.2, between about 5.0 to about 6.3, between about 5.0 to about 6.4, between about 5.1 to about 6.5, between about 5.2 to about 6.5, between about 5.3 to about 6.5, or between about 5.4 to about 6.5.

In some aspects, the original pH of the culture comprising the anaerobic cells (e.g., M. elsdenii cells) at the time of harvesting is about 6.0. In some aspects, the pH is increased to about 7.5 for about 5 minutes to about 60 minutes. In some aspects, the pH is decreased to about 3.5 for about 5 minutes to about 60 minutes. In some aspects, after the about 5 minutes to 60 minutes, the pH is adjusted back to the original pH of about 6.0. In some aspects, the adjustment back to the original pH is conducted with an osmoprotectant. In some aspects, the osmoprotectant is selected from a group consisting of betaine, trehalose, proline, ectoine, or combinations thereof. In some aspects, the osmoprotectant is a sugar, a sugar alcohol, a sugar derivative, a polysaccharide, an encapsulating polymer, or a nitrogen source, and a mixture thereof. In some aspects the sugar is sucrose. In some aspects, the adjustment back to the original pH is conducted without an osmoprotectant.

In some aspects, the pH is increased to about 7.5 for about 5 minutes, about 10 minutes, about 15 minutes, about 20 minutes, about 25 minutes, about 30 minutes, about 35 minutes, about 40 minutes, about 45 minutes, about 50 minutes, about 55 minutes, or about 60 minutes. In some aspects, the pH is increased to about 7.5 for about 5 minutes to about 60 minutes, about 10 minutes to about 60 minutes, about 15 minutes to about 60 minutes, about 20 minutes to about 60 minutes, about 25 minutes to about 60 minutes, about 30 minutes to about 60 minutes, about 35 minutes to about 60 minutes, about 45 minutes to about 60 minutes, about 5 minutes to about 45 minutes, about 5 minutes to about 30 minutes, about 5 minutes to about 25 minutes, about 5 minutes to about 20 minutes, about 5 minutes to about 15 minutes, or about 5 minutes to about 10 minutes.

In some aspects, the pH is decreased to about 3.5 for about 5 minutes, about 10 minutes, about 15 minutes, about 20 minutes, about 25 minutes, about 30 minutes, about 35 minutes, about 40 minutes, about 45 minutes, about 50 minutes, about 55 minutes, or about 60 minutes. In some aspects, the pH is decreased to about 3.5 for about 5 minutes to about 60 minutes, about 10 minutes to about 60 minutes, about 15 minutes to about 60 minutes, about 20 minutes to about 60 minutes, about 25 minutes to about 60 minutes, about 30 minutes to about 60 minutes, about 35 minutes to about 60 minutes, about 45 minutes to about 60 minutes, about 5 minutes to about 45 minutes, about 5 minutes to about 30 minutes, about 5 minutes to about 25 minutes, about 5 minutes to about 20 minutes, about 5 minutes to about 15 minutes, or about 5 minutes to about 10 minutes.

In some aspects, the method comprises inoculating growth media in a fermenter with an inoculum comprising anaerobic cells (e.g., M. elsdenii cells) to prepare a culture, and incubating the culture at a temperature of about 39° C. until the pH of the culture is about 6.0. In some aspects, the inoculum comprising anaerobic cells (e.g., M. elsdenii). In some aspects, the method comprises inoculating growth media in a fermenter an inoculum to media ratio of 1/50 to 1/4,000. In some aspects, the inoculum to media ratio is 1/100.

Electrospray Drying Anaerobic Bacterial Cells and/or M. elsdenii Cells

Spray drying is a method of producing a dry powder from a liquid or slurry by rapidly drying with a hot gas (typically air or nitrogen).

The spray drying process starts with a liquid solvent, usually water, containing dissolved or suspended components such as an emulsion (for example, an emulsion containing anaerobic cells and/or M. elsdenii). The suspension includes a substance to be encapsulated (the load) and an amphipathic carrier (typically a modified starch), which are homogenized as a suspension in the liquid solvent. The load is anaerobic cells and/or M. elsdenii, and the homogenized suspension is often referred to as a slurry.

Spray dryers use a type of atomizer, such as a spray nozzle, to disperse the slurry into a controlled spray having some relatively controlled droplet size. Depending on the process requirements, droplet sizes may range from about 10 to 500 microns in diameter. The most common applications require droplet sizes in the 50 to 200 micron range.

In conjunction with atomization, the slurry is fed into a drying chamber, usually a tower into which heated air is also introduced. The temperature of the air as it enters the drying chamber is usually in the range of 180-200° C. The heated air supplies energy for evaporation of volatile components of the liquid (the water) from the droplets. As the water evaporates, the carrier forms a hardened shell around the load, producing a dried powder.

The anaerobic cells (e.g., M. elsdenii) are either emulsified in the carrier fluid system or dissolved into it to form a slurry. In some aspects, the anaerobic cells are selected from the group consisting of: Bifidobacterium cells, such as B. breve, Lactobacillus cells, such as L. plantarum, Bifidobacterium cells, such as B. animalis subsp. lactis, Pediococcus cells, such as P. acidilactici, Lactobacillus cells, such as L. casei, Fibrobacter, such as F. succinogenes, Ruminococcus, such as R. flavefaciens, and Butyrivibrio, such as B. fibrisolvens.

The slurry formed in a solution tank and is delivered to an atomizer using a pump or other means of conveyance. The slurry enters the atomizer and exits the atomizer as a spray of liquid droplets, and the droplets are introduced into a drying chamber. A feed of drying fluid is heated by a process heater and supplied into the drying chamber by a blower. In some aspects, the drying fluid is air, argon, or nitrogen. The water evaporated from the droplets enters the heated air as the atomized liquid droplets dry to form solid particles after exposure to the incoming heated air.

The dried powder leaves the dryer chamber along with the water vapor laden air, and is carried to a cyclone separator, which removes the dried particles from the circulating air stream and deposits the particles into a collection container. The water vapor laden air exits the collection container and enters a baghouse, where very fine particles are removed before the water vapor laden air is sent into a condenser, via a blower. The condenser removes the water vapor from the process air, and the collected water may be re-used or discarded.

One of the prominent attributes of the traditional spray drying process is the high temperature of the inlet gas (approximately 200° C.) leaving the heater and entering the drying chamber, as well as the temperature of the outlet gas exiting the drying chamber, which is usually greater than 100° C. Although the liquid droplets are injected into the high temperature environment within the chamber, the droplets do not actually reach the inlet gas temperature. The droplets, however, do become heated to a point at which considerable portions of desired constituents of the droplets (i.e., portions of the load-anaerobic bacterial cells and/or M. elsdenii) are undesirably modified. The undesirable modification to the load (load loss) leads to a reduction in viability of the anaerobic cells and/or M. elsdenii. Thus, evaporation and heat degradation of the load lowers the performance characteristics of the final powder product, and therefore results in a significant degradation of performance in commercial use and a significant loss of revenue.

The present disclosure provides a method of electrospray drying, using a low temperature, e.g., “no heat”, spray drying process that produces powder bacterial products (e.g., anaerobic cells and/or M. elsdenii) with superior viability and stability. The spray drying method of the present disclosure does not employ heated gas for removing water from the atomized fluid droplets, as has been the case in previously employed spray drying operations. The spray drying method of the present disclosure instead uses unheated air, e.g., dehumidified air, to carry out high throughput atomization processes, utilizing unique dryer designs and high solids content (low water content) slurries/emulsions with viscosities in the range of 1-2000 cP to produce powders dried at low temperatures, such as temperatures on the order of from 50 to 100° C. The spray drying method of the present disclosure instead uses unheated air, nitrogen, or argon, e.g., dehumidified air, nitrogen, or argon, to carry out high throughput atomization processes, utilizing unique dryer designs and high solids content (low water content) slurries/emulsions with viscosities in the range of 1-2000 cP to produce powders dried at low temperatures, such as temperatures on the order of from 50 to 100° C. Additionally, an electrostatic charge is applied to the slurry or to the atomized droplets, whereby the respective charge on the wet droplets produces a force that causes adjacent droplets to repel one another.

In some aspects, the present invention is directed to a method of electrospray drying anaerobic cells and/or M. elsdenii cells.

In some aspects, the present invention is directed to electrospray dried anaerobic cells and/or M. elsdenii cells.

In some aspects, the present invention is directed to electrospray dried anaerobic cells and/or M. elsdenii cells produced by a method or a system disclosed herein.

In some aspects, the disclosure provides a system for spray drying a liquid product into a dried powder without employing heated air or using low heated air in accordance with one or more aspects of the present invention. In some aspects, the disclosure provides a system for spray drying a liquid product into a dried powder without employing heated air, nitrogen, or argon or using low heated air, nitrogen, or argon in accordance with one or more aspects of the present invention. The system includes some of the same or similar elements as in the conventional spray drying system.

In some aspects, the system includes a drying chamber into which a slurry comprising anaerobic cells and/or M. elsdenii is fed by way of a pump (or equivalent conveying mechanism). The slurry enters an atomizer and leaves the atomizer as a spray of liquid droplets, which are introduced into the drying chamber. A feed of non-heated fluid or low-heated fluid (such as air, nitrogen, or argon or another suitable gas) is supplied into the drying chamber by a blower. The supplied air, nitrogen, or argon may be subjected to dehumidification (via dehumidifier) prior to introduction into the drying chamber. The atomized liquid droplets dry to form solid particles after exposure to the incoming air, nitrogen, or argon. Water evaporates from the droplets and enters the air, nitrogen, or argon within the drying chamber. Dried powder leaves the drying chamber along with the water vapor laden air, nitrogen, or argon, and is carried to a cyclone separator, which removes the dried particles from the circulating air, nitrogen, or argon stream and deposits the particles into a collection container. The water vapor laden air, nitrogen, or argon exits the collection container and enters a baghouse, where very fine particles are removed before the water vapor laden air, nitrogen, or argon is sent into a condenser, via a blower. The condenser removes the water vapor from the process air, nitrogen, or argon, and the collected water/gas may be re-used or discarded.

Another reason that non-heated or low-heated air, nitrogen, or argon may be used in the spray drying system and process is that the slurry is not conventional. In some aspects, the slurry includes a liquid solvent, a carrier, and an active organism. In some aspects, the liquid solvent is water, however, other suitable solvents may be employed if needed or desired. In some aspects, the carrier is a carbohydrate. In some aspects, the active organism may be anaerobic bacteria and/or M. elsdenii.

Formation of the slurry is controlled such that regulation of at least one variable such as viscosity, the amount of liquid solvent (e.g., water), or other suitable metric relating to the water content of the slurry, is obtained. In some aspects, the formation of the slurry may include controlling a viscosity of the slurry such that the viscosity at the atomization step is between about 1-2000 centipoise (cP). In some aspects, the viscosity at the atomization step is between about 10-1950 centipoise (cP). In some aspects, the viscosity at the atomization step is between about 50-1900 centipoise (cP). In some aspects, the viscosity at the atomization step is between about 100-1800 centipoise (cP). In some aspects, the viscosity at the atomization step is between about 150-1750 centipoise (cP). In some aspects, the viscosity at the atomization step is between about 200-1700 centipoise (cP). In some aspects, the viscosity at the atomization step is between about 250-1650 centipoise (cP). In some aspects, the viscosity at the atomization step is between about 300-1600 centipoise (cP). In some aspects, the viscosity at the atomization step is between about 350-1550 centipoise (cP). In some aspects, the viscosity at the atomization step is between about 400-1500 centipoise (cP). In some aspects, the viscosity at the atomization step is between about 450-1450 centipoise (cP). In some aspects, the viscosity at the atomization step is between about 500-1400 centipoise (cP). In some aspects, the viscosity at the atomization step is between about 550-1350 centipoise (cP). In some aspects, the viscosity at the atomization step is between about 600-1300 centipoise (cP). In some aspects, the viscosity at the atomization step is between about 650-1250 centipoise (cP). In some aspects, the viscosity at the atomization step is between about 700-1200 centipoise (cP). In some aspects, the viscosity at the atomization step is between about 750-1150 centipoise (cP). In some aspects, the viscosity at the atomization step is between about 800-1100 centipoise (cP). In some aspects, the viscosity at the atomization step is between about 850-1050 centipoise (cP). In some aspects, the viscosity at the atomization step is between about 900-1000 centipoise (cP). In some aspects, the viscosity at the atomization step is between about 925-975 centipoise (cP). In some aspects, the viscosity at the atomization step is between 1-500 cP. In some aspects, the viscosity at the atomization step is 5-50 centipoise (cP).

In some aspects, the formation of the slurry may include controlling a ratio of water or liquid solvent within the slurry such that the ratio of water or liquid solvent within the slurry at the atomization step is at least one of: between about 70-99 weight percentage; between about 71-98 weight percentage; between about 72-97 weight percentage; between about 73-96 weight percentage; between about 74-95 weight percentage; between about 75-95 weight percentage; between about 76-94 weight percentage; between about 77-93 weight percentage; between about 78-92 weight percentage; between about 79-91 weight percentage; between about 80-89 weight percentage; between about 81-88 weight percentage; between about 82-87 weight percentage; between about 83-86 weight percentage; and between about 84-85 weight percentage.

Another reason non-heated air, nitrogen, or argon can be used in the spray drying system and process is because electrostatic charging process is conducted before, during or after atomization. In particular, an electrostatic charge is applied to the slurry or to the atomized droplets. In this regard, the system and/or process includes a high voltage supply (about 0.1-45 kV DC) that is coupled to one or more electrodes. In some aspects, the high voltage supply is about 0.5-40 kV DC. In some aspects, the high voltage supply is about 1-35 kV DC. In some aspects, the high voltage supply is about 5-30 kV DC. In some aspects, the high voltage supply is about 10-25 kV DC. In some aspects, the high voltage supply is about 15-20 kV DC. In some aspects, the high voltage supply is about 15 kV DC.

In some aspects, the system and/or process includes a programmable profile using a control module to provide a voltage gradient. In some aspects, the voltage gradient begins at about 0.1 kV and increases to 45 kV. In some aspects, the voltage gradient begins at about 0.5 kV and increases to 40 kV. In some aspects, the voltage gradient begins at about 1 kV and increases to 35 kV. In some aspects, the voltage gradient begins at about 5 kV and increases to 30 kV. In some aspects, the voltage gradient begins at about 10 kV and increases to 25 kV. The use of a voltage gradient will, for example, contort the magnetic field creating particle structures that have high viability and/or stability.

In some aspects, the system and/or process includes a programmable profile using a control module to provide an oscillating voltage. In some aspects, the voltage will oscillate between 0.1 kV to 45 kV, with a set point of 25 kV. In some aspects, the voltage will oscillate between 1 kV to 40 kV, with a set point of 25 kV. In some aspects, the voltage will oscillate between 5 kV to 35 kV, with a set point of 25 kV. In some aspects some aspects, the voltage will oscillate between 10 kV to 30 kV, with a set point of 25 kV. The use of an oscillating voltage will, for example, contort the magnetic creating particle structures that have high viability and/or stability.

The polarity of the high voltage supply may be in either the positive or negative configuration. The slurry (or atomized droplets) is brought into contact with the electrode to impart an electrical charge thereto. In a preferred aspect, the slurry is brought into contact with the electrode(s) in order to produce a charged slurry. Concurrently or thereafter, the charged slurry is atomized to produce a plurality of electrostatically charged, wet particles (droplets).

The respective charge on the wet droplets produces a force that tends to cause adjacent droplets to repel one another. Additionally, the force on a given droplet opposes the surface tension of such given droplet. When the charge on the given droplet exceeds a threshold level, the Rayleigh limit, the droplet becomes unstable and smaller satellite droplets are ejected from the given (parent) droplet. One or more of the satellite droplets, in turn, might also become unstable and produce additional satellite droplets, since the surface charge density does not diminish in the satellite droplets as evaporation takes place.

The electrostatically charged, wet particles/droplets are suspended for a sufficient time within the drying chamber to permit the aforementioned repulsive forces induced by the electrostatic charge on at least some wet particles/droplets to cause at least some of such particles to divide into wet sub-particles/droplets. The suspension of the droplets continues, without the presence of any heated drying fluids, for a sufficient time to drive off a sufficient amount of the liquid solvent within most of the wet particles/droplets to leave a plurality of dried particles (the powder), each dried particle containing the active organism encapsulated within the carrier. Notably, the production of sub-particles/droplets from a given volume of atomized slurry (i.e., from a given droplet) results in faster drying of such volume due to a greatly increased aggregate surface area of the sub-particles/droplets and concomitant reduction of particle volume of each sub-particle/droplet following each fission event.

The production of sub-particles/droplets can be called coulombic fission. The time scale for such coulombic fission events is on the order of a few microseconds to milliseconds. The fission of about ten sub-particles/droplets from a given particle/droplet reduces a diameter of the given particle/droplet by about 30%. The amount of time that it takes to achieve such reduction in diameter (on the order of a few microseconds to milliseconds) is an order of magnitude faster than diffusive evaporation in the presence of heated air, nitrogen, or argon, which occurs with a characteristic time t in accordance with the following formula: t=do²/k where do is the diameter of the particle and k is the evaporative diffusion coefficient. For particles in the 20 to 200 mm diameter range, the time to any significant diameter reduction by evaporation is on the order of tenths to several seconds, which is far longer (one to two orders of magnitude) than diameter reduction by coulombic fission.

The individual or combined characteristics of relative low water content in the slurry and electrostatic charge on the droplets permits a vastly lower temperature condition within the drying chamber as compared with heated air systems and processes. In some aspects, the temperature of the non-heated drying fluid (e.g., air) introduced into the drying chamber is between 15-100° C. In some aspects, the temperature of the non-heated drying fluid (e.g., air) introduced into the drying chamber is between 20-95° C. In some aspects, the temperature of the non-heated drying fluid (e.g., air) introduced into the drying chamber is between 25-90° C. In some aspects, the temperature of the non-heated drying fluid (e.g., air) introduced into the drying chamber is between 30-85° C. In some aspects, the temperature of the non-heated drying fluid (e.g., air) introduced into the drying chamber is between 35-80° C. In some aspects, the temperature of the non-heated drying fluid (e.g., air) introduced into the drying chamber is between 40-75° C. In some aspects, the temperature of the non-heated drying fluid (e.g., air) introduced into the drying chamber is between 45-70° C. In some aspects, the temperature of the non-heated drying fluid (e.g., air) introduced into the drying chamber is between 50-70° C. In some aspects, the temperature of the non-heated drying fluid (e.g., air) introduced into the drying chamber is between 55-65° C. In some aspects, the temperature of the non-heated drying fluid (e.g., air) introduced into the drying chamber is 62.5° C. In some aspects, the temperature of the non-heated drying fluid (e.g., air) introduced into the drying chamber is 67.5° C. In some aspects, the temperature of the non-heated drying fluid (e.g., air) introduced into the drying chamber is 70° C. In some aspects, the temperature of the non-heated drying fluid (e.g., air) introduced into the drying chamber is 80° C. In some aspects, the temperature of the non-heated drying fluid (e.g., air) introduced into the drying chamber is 90° C.

In some aspects, the temperature of the non-heated drying fluid (e.g., air, nitrogen, or argon) introduced into the drying chamber is between 15-100° C. In some aspects, the temperature of the non-heated drying fluid (e.g., air, nitrogen, or argon) introduced into the drying chamber is between 20-95° C. In some aspects, the temperature of the non-heated drying fluid (e.g., air, nitrogen, or argon) introduced into the drying chamber is between 25-90° C. In some aspects, the temperature of the non-heated drying fluid (e.g., air, nitrogen, or argon) introduced into the drying chamber is between 30-85° C. In some aspects, the temperature of the non-heated drying fluid (e.g., air, nitrogen, or argon) introduced into the drying chamber is between 35-80° C. In some aspects, the temperature of the non-heated drying fluid (e.g., air, nitrogen, or argon) introduced into the drying chamber is between 40-75° C. In some aspects, the temperature of the non-heated drying fluid (e.g., air, nitrogen, or argon) introduced into the drying chamber is between 45-70° C. In some aspects, the temperature of the non-heated drying fluid (e.g., air, nitrogen, or argon) introduced into the drying chamber is between 50-70° C. In some aspects, the temperature of the non-heated drying fluid (e.g., air, nitrogen, or argon) introduced into the drying chamber is between 55-65° C. In some aspects, the temperature of the non-heated drying fluid (e.g., air, nitrogen, or argon) introduced into the drying chamber is 62.5° C. In some aspects, the temperature of the non-heated drying fluid (e.g., air, nitrogen, or argon) introduced into the drying chamber is 67.5° C. In some aspects, the temperature of the non-heated drying fluid (e.g., air, nitrogen, or argon) introduced into the drying chamber is 70° C. In some aspects, the temperature of the non-heated drying fluid (e.g., air, nitrogen, or argon) introduced into the drying chamber is 80° C. In some aspects, the temperature of the non-heated drying fluid (e.g., air, nitrogen, or argon) introduced into the drying chamber is 90° C.

In some aspects, the drying fluid is a gas. In some aspects, the drying fluid is argon. In some aspects, the drying fluid is nitrogen. In some aspects, the drying fluid is air.

Typically, the inlet air, nitrogen, or argon temperature will result in an outlet air temperature from the drying chamber that is lower than the inlet air, nitrogen, or argon temperature.

In some aspects, the method further comprises heating the air to between 50° C. to 100° C. by applying a voltage of 0.1-45 kV prior to feeding the air into the drying chamber. In some aspects, the method further comprises heating the air to between about 40° C. to about 120° C. by applying a voltage of 0.1-45 kV prior to feeding the air into the drying chamber. In some aspects, the method further comprises heating the air by applying a voltage prior to feeding the air into the drying chamber. In some aspects, the air is heated to between about 40° C. to about 120° C., between about 40° C. to about 110° C., between about 40° C. to about 100° C., between about 40° C. to about 90° C., between about 40° C. to about 80° C., between about 40° C. to about 70° C. between about 40° C. to about 60° C., between about 40° C. to about 50° C., between about 50° C. to about 120° C., between about 60° C. to about 120° C., between about 70° C. to about 120° C., between about 80° C. to about 120° C., between about 90° C. to about 120° C., between about 100° C. to about 120° C., or between about 110° C. to about 120° C. In some aspects, the method further comprises heating the air to between 50° C. to 100° C. by applying a voltage of 0.1-45 kV prior to feeding the air, nitrogen, or argon into the drying chamber. In some aspects, the method further comprises heating the air, nitrogen, or argon to between about 40° C. to about 120° C. by applying a voltage of 0.1-45 kV prior to feeding the air, nitrogen, or argon into the drying chamber. In some aspects, the method further comprises heating the air, nitrogen, or argon by applying a voltage prior to feeding the air, nitrogen, or argon into the drying chamber. In some aspects, the air, nitrogen, or argon is heated to between about 40° C. to about 120° C., between about 40° C. to about 110° C., between about 40° C. to about 100° C., between about 40° C. to about 90° C., between about 40° C. to about 80° C., between about 40° C. to about 70° C. between about 40° C. to about 60° C., between about 40° C. to about 50° C., between about 50° C. to about 120° C., between about 60° C. to about 120° C., between about between about 60° C. to about 90° C., between about 70° C. to about 120° C., between about 80° C. to about 120° C., between about 90° C. to about 120° C., between about 100° C. to about 120° C., or between about 110° C. to about 120° C. In some aspects, the voltage is about 0.1 kV, about 1 kV, about 5 kV, about 10 kV, about 15 kV, about 20 kV, about 25 kV, about 30 kV, about 35 kV, about 40 kV, or about 45 kV. In some aspects, the voltage is between about 0.1 to about 45 kV. In some aspects, the voltage is between about 0.1 to about 45 kV, about 0.1 to about 40 kV, about 0.1 to about 35 kV, about 0.1 to about 30 kV, about 0.1 to about 25 kV, about 0.1 to about 20 kV, about 0.1 to about 15 kV, about 0.1 to about 10 kV, about 0.1 to about 5 kV, about 0.1 to about 1 kV, about 1 to about 45 kV, about 5 to about 45 kV, about 10 to about 45 kV, about 15 to about 45 kV, about 20 to about 45 kV, about 25 to about 45 kV, about 30 to about 45 kV, about 35 to about 45 kV, or about 40 to about 45 kV.

In some aspects, the method further comprises heating the air to between 50° C. to 100° C. by applying a voltage of 15 kV prior to feeding the air into the drying chamber. In some aspects, the method further comprises heating the air, nitrogen, or argon to between 50° C. to 100° C. by applying a voltage of 15 kV prior to feeding the air, nitrogen, or argon into the drying chamber.

In some aspects, the air is at a temperature between 70° C. to 90° C. In some aspects, the air, nitrogen, or argon is at a temperature between 70° C. to 90° C.

In some aspects, the method further comprises heating the air to between 70° C. to 90° C. by applying a voltage of 15 kV prior to feeding the air into the drying chamber. In some aspects, the method further comprises heating the air, nitrogen, or argon to between 70° C. to 90° C. by applying a voltage of 15 kV prior to feeding the air, nitrogen, or argon into the drying chamber.

In some aspects, the voltage is applied constantly or in pulses.

In some aspects, it may be desirable to ensure that the drying fluid (e.g., air, nitrogen, or argon) introduced into the drying chamber is of relatively low water content. Thus, the system may include a process dehumidifier in order to remove some amount of water from the drying fluid prior to introduction into the drying chamber. In some aspects, after dehumidification, the non-heated air, nitrogen, or argon inputted into the drying chamber may be at a relative humidity of about between 1-20%. In some aspects, after dehumidification, the non-heated air, nitrogen, or argon inputted into the drying chamber may be at a relative humidity of about between 2-19%. In some aspects, after dehumidification, the non-heated air, nitrogen, or argon inputted into the drying chamber may be at a relative humidity of about between 3-18%. In some aspects, after dehumidification, the non-heated air, nitrogen, or argon inputted into the drying chamber may be at a relative humidity of about between 4-17%. In some aspects, after dehumidification, the non-heated air, nitrogen, or argon inputted into the drying chamber may be at a relative humidity of about between 5-16%. In some aspects, after dehumidification, the non-heated air, nitrogen, or argon inputted into the drying chamber may be at a relative humidity of about between 6-15%. In some aspects, after dehumidification, the non-heated air, nitrogen, or argon inputted into the drying chamber may be at a relative humidity of about between 7-14%. In some aspects, after dehumidification, the non-heated air, nitrogen, or argon inputted into the drying chamber may be at a relative humidity of about between 8-13%. In some aspects, after dehumidification, the non-heated air, nitrogen, or argon inputted into the drying chamber may be at a relative humidity of about between 9-12%. In some aspects, after dehumidification, the non-heated air, nitrogen, or argon inputted into the drying chamber may be at a relative humidity of about between 10-11%.

In some aspects, the dewpoint of the non-heated air, nitrogen, or argon may be in a range of from −20° C. to 5° C., from −15° C. to 5° C., from −12° C. to 3° C., from −12° C. to 0° C., from −12° C. to −5° C., or any other suitable dewpoint range appropriate to the spray drying operation.

The atomizer may be implemented by way of any of the known methods, apparatus, and/or techniques. In some aspects, the atomizer may be implemented using at least one of: a nozzle technique, a centrifugal technique, a pneumatic technique, and an ultrasonic technique. For most atomization techniques, the slurry does not leave the atomizing mechanism as a final droplet, but rather as a fragment of a thin liquid film or ligament. The formation of droplets takes place immediately after the liquid has left the atomizing mechanism, due to the surface tension of the liquid. The droplet size from a given type of atomization depends on the energy input into breaking the slurry into fragments to, for example, increase the overall effective surface area of the slurry.

In some aspects, the average droplet size and distribution may be fairly constant for a given atomization technique, and is in the range of 1-500 microns. In some aspects, the average droplet size is in the range of 10-450 microns. In some aspects, the average droplet size is in the range of 50-400 microns. In some aspects, the average droplet size is in the range of 100-350 microns. In some aspects, the average droplet size is in the range of 150-300 microns. In some aspects, the average droplet size is in the range of 200-250 microns.

In some aspects, an atomizer with a single nozzle (i.e., single fluid) will encapsulate the cells by dispensing and mixing the cells, the carrier, and encapsulating material. In some aspects, an atomizer with two nozzles (i.e., dual fluid) will dispense the cells from one nozzle and the carrier and/or encapsulating material in a second nozzle, and mix the two fluids together to encapsulate the cells (by creating a core and outer layer coating). In some aspects, an atomizer with a single nozzle (i.e., single fluid) will spray dry the cells mixed with the carrier, and an encapsulation step will be performed following spray drying or Wurster coater or other coating equipment.

The electrostatic charge process and resultant coulombic fission process in accordance with the various aspects herein produces, in general, larger particles than conventional spray drying processes. The larger particles, however, come from even larger, parent particles, which conventional atomizers cannot adequately produce. The daughter particles produced in accordance with the aspects herein are smaller, and the process tends to make bimodal size distributions for very viscous slurries.

Centrifugal (or rotary) atomization may be considered the most common form of atomization. Centrifugal atomization employs a rotating disc or wheel, which breaks the liquid stream of slurry into droplets. In some aspects, the centrifugal atomization device may employ a disc or wheel of about 5 to 50 cm in diameter. In some aspects, the centrifugal atomization device may employ a disc or wheel of about 10 to 45 cm in diameter. In some aspects, the centrifugal atomization device may employ a disc or wheel of about 10 to 45 cm in diameter. In some aspects, the centrifugal atomization device may employ a disc or wheel of about 15 to 40 cm in diameter. In some aspects, the centrifugal atomization device may employ a disc or wheel of about 20 to 30 cm in diameter. In some aspects, the disc or wheel can spin in the range of about 5,000 to 40,000 rpm. In some aspects, the disc or wheel can spin in the range of about 10,000 to 35,000 rpm. In some aspects, the disc or wheel can spin in the range of about 15,000 to 30,000 rpm. In some aspects, the disc or wheel can spin in the range of about 20,000 to 30,000 rpm. The size of the droplets produced by a centrifugal atomization device is about inversely proportional to the peripheral speed of the disc or wheel.

Nozzle atomization employs a pump, which pressurizes and forces the slurry through the orifice of a nozzle to break the liquid into fine droplets. In some aspects, the orifice size is usually in the range of 0.5 to 30 mm. In some aspects, the orifice size is usually in the range of 1 to 29 mm. In some aspects, the orifice size is usually in the range of 5 to 25 mm. In some aspects, the orifice size is usually in the range of 10 to 20 mm. In some aspects, the orifice size is usually in the range of 13 to 17 mm. The size of the droplets depends on the size of the orifice and the pressure drop. A larger pressure drop across the orifice produces smaller droplets. Therefore, to reduce the particle/droplet size for a given feed rate, a smaller orifice and a higher pump pressure may be employed.

Two-fluid pneumatic atomization employs the interaction of the slurry with another fluid, typically compressed air using a fluid nozzle for the compressed air and a fluid nozzle for the slurry. In some aspects, the pressure of the air and slurry may be in the range of about 200 to 350 kPa. In some aspects, the pressure of the air and slurry may be in the range of about 210 to 340 kPa. In some aspects, the pressure of the air and slurry may be in the range of about 220 to 330 kPa. In some aspects, the pressure of the air and slurry may be in the range of about 230 to 320 kPa. In some aspects, the pressure of the air and slurry may be in the range of about 240 to 310 kPa. In some aspects, the pressure of the air and slurry may be in the range of about 250 to 300 kPa. In some aspects, the pressure of the air and slurry may be in the range of about 260 to 290 kPa. In some aspects, the pressure of the air and slurry may be in the range of about 270 to 280 kPa. Particle size is controlled by varying a ratio of the compressed air flow to that of the slurry flow. In some aspects, two-fluid pneumatic atomization employs the interaction of the slurry with another fluid, typically compressed air, nitrogen, or argon using a fluid nozzle for the compressed air, nitrogen, or argon and a fluid nozzle for the slurry. In some aspects, the pressure of the air, nitrogen, or argon and slurry may be in the range of about 200 to 350 kPa. In some aspects, the pressure of the air, nitrogen, or argon and slurry may be in the range of about 210 to 340 kPa. In some aspects, the pressure of the air, nitrogen, or argon and slurry may be in the range of about 220 to 330 kPa. In some aspects, the pressure of the air, nitrogen, or argon and slurry may be in the range of about 230 to 320 kPa. In some aspects, the pressure of the air, nitrogen, or argon and slurry may be in the range of about 240 to 310 kPa. In some aspects, the pressure of the air, nitrogen, or argon and slurry may be in the range of about 250 to 300 kPa. In some aspects, the pressure of the air, nitrogen, or argon and slurry may be in the range of about 260 to 290 kPa. In some aspects, the pressure of the air, nitrogen, or argon and slurry may be in the range of about 270 to 280 kPa. Particle size is controlled by varying a ratio of the compressed air, nitrogen, or argon flow to that of the slurry flow.

Sonic atomization employs ultrasonic energy to vibrate a surface at ultrasonic frequencies. The slurry is brought into contact with the vibrating surface in order to produce the particles/droplets.

In some aspects, the present disclosure may use a centrifugal atomizer, a nozzle-type atomizer, a two-fluid atomizer, or a sonic atomizer.

In some aspects, the two-fluid atomizer comprises a body having a proximal end and a distal end. In some aspects, the two-fluid atomizer has a channel that extends through the body and includes an inlet, typically near the proximal end of the body, and an outlet, generally near the distal end of the body. The channel operates to convey a first of the two-fluids, i.e., the slurry, from the inlet to the outlet.

In some aspects, the two-fluid atomizer also includes at least one electrode operating to contact the slurry and apply an electrostatic charge thereto, such that the two-fluid atomizer operates to produce a plurality of electrostatically charged wet particles/droplets. In some aspects, the at least one electrode may be disposed within the body of the two-fluid atomizer such that the slurry contacts the electrode and becomes electrostatically charged while flowing from the inlet to the outlet of the channel. In some aspects, the electrode may be disposed within the channel, preferably in a coaxial arrangement, such that a significant portion of the surface area of the electrode is available for contact with the slurry. In some aspects, the electrode may be inserted into the channel by way of a threaded bore of the body and complementary threaded shaft of the electrode, which when engaged, positions the electrode within the channel. In some aspects, a connection terminal may be electrically and mechanically coupled to the electrode in order to provide a means for connecting with the high voltage supply and receiving voltage potential at the surface of the electrode.

In some aspects, the two-fluid atomizer may include a nozzle in fluid communication with the outlet of the channel. In some aspects, the outlet of the channel includes a tube sized and shaped to engage, and be received within, a complementary bore at an inlet end of the nozzle. The engagement of the tube and bore permits fluid communication of the slurry (which has been electrostatically charged) from the channel into an internal volume intermediately disposed within the nozzle. In some aspects, a sealing ring may be employed to ensure a fluid tight seal between the tube and the bore, even under fluid pressure. In some aspects, the nozzle preferably includes a transition section of reducing diameter (a tapering surface) extending from the internal volume to a nozzle orifice. In some aspects, the nozzle orifice is preferably of a generally cylindrical shape, including an internal bore of a size sufficient to produce wet particles/droplets of desired size and shape once they succumb to surface tension forces.

In some aspects, the two-fluid atomizer may further include a nozzle cap, which generally surrounds the nozzle and permits the nozzle orifice to extend through a bore at a distal end thereof. In some aspects, the nozzle cap includes an engagement feature at a proximal end thereof, which engages the distal end of the body. In some aspects, the nozzle cap includes a threaded shank, which threads into a complementary threaded bore of the body. In some aspects, a sealing ring may be employed to ensure a fluid tight seal as between an internal surface of the nozzle cap and an external surface of the nozzle, thereby forming an internal volume between.

In some aspects, the two-fluid atomizer includes another channel extending through the body, which includes an inlet, typically near the proximal end of the body, and an outlet, typically near the distal end of the body. In some aspects, the channel operates to convey a second of the two-fluids, i.e., the non-heated air, nitrogen, or argon, from the inlet to the outlet. In some aspects, the outlet is in fluid communication with the internal volume (between the internal surface of the nozzle cap and the external surface of the nozzle). Thus, the channel operates to convey the non-heated air, nitrogen, or argon from the proximal end to the distal end of the two-fluid atomizer. In some aspects, the flow of the non-heated air, nitrogen, or argon through the two-fluid atomizer may be about 5,100 m³/h at an input pressure of about 130 psi. In some aspects, the flow of the non-heated air, nitrogen, or argon through the two-fluid atomizer may be about 4,000 m³/h at an input pressure of about 100 psi. In some aspects, the flow of the non-heated air, nitrogen, or argon through the two-fluid atomizer may be about 4,500 m³/h at an input pressure of about 115 psi. In some aspects, the flow of the non-heated air, nitrogen, or argon through the two-fluid atomizer may be about 6,200 m³/h at an input pressure of about 160 psi. In some aspects, the flow of the non-heated air, nitrogen, or argon through the two-fluid atomizer may be about 5,500 m³/h at an input pressure of about 145 psi.

In some aspects, the nozzle includes a tapered surface on an exterior thereof, which is downstream of the exterior surface and downstream of the internal volume. In some aspects, the nozzle cap includes a complementary internal surface in abutment with the tapered surface. A number of grooves (recesses) are disposed in the tapered surface and extend from the internal volume toward the nozzle orifice. When the complementary internal surface of the nozzle cap is in abutment with the tapered surface, the grooves provide fluid communication of the non-heated air, nitrogen, or argon from the internal volume toward the nozzle orifice. The grooves terminate at an annular space between a peripheral edge of the tapered surface and the outer surface of the nozzle orifice, where the nozzle orifice exits the nozzle. The annular space is in fluid communication with the bore, whereby a suitably sized bore (larger than a diameter of the nozzle orifice) permits the non-heated air, nitrogen, or argon to exit the nozzle and nozzle cap under pressure. In some aspects, the grooves extend such that they terminate tangentially to the annular space and thereby cause the non-heated air, nitrogen, or argon to produce a swirling fluid motion within the space and in the vicinity of the nozzle orifice after it has exited the bore.

In some aspects, the swirling fluid motion of the non-heated air, nitrogen, or argon, as it leaves the nozzle and nozzle cap, imparts a swirling agitation to the plurality of wet particles/droplets as they leave the nozzle. This swirling agitation may suspend and agitate the wet particles/droplets in order to achieve the aforementioned fission and evaporation. In some aspects, the above approach to atomization enables relatively high slurry throughput, on the order of 1-20 kg/h at an input pressure of about 20-100 psi. In some aspects, the above approach to atomization enables relatively high slurry throughput, on the order of 0.1-25 kg/h at an input pressure of about 15-125 psi.

In some aspects, a drying chamber may be employed in the system in accordance with the present disclosure. In some aspects, the drying chamber can include an inlet end, an outlet end, and an internal volume, within which the wet particles/droplets are dried. In some aspects, the drying chamber is formed from a non-electrically conductive material. In some aspects, the choice of materials would be a non-metal to avoid electrically conductive material. However, the conventional wisdom of the prior art spray drying process requires heated air (on the order of 200° C.), which consequently requires a metal drying chamber (typically stainless steel), otherwise the chamber would warp or otherwise fail.

In some aspects, using non-heated air, nitrogen, or argon allows the drying chamber to be formed from a non-metallic material as the temperature inside the drying chamber could be less than 50° C. In some aspects, the drying chamber could be formed of materials such as polymer-based composites. Filament wound fiberglass composite tanks (which are used for storage of water, various food stuffs, grain storage, brines and many non-food based applications) have excellent load carrying properties and can be used for making very large tanks. In some aspects, filament wound fiberglass composite materials may be used to fabricate the drying chamber disclosed herein. Engineered plastics typically has a lower cost for the basic materials and the cost of manufacturing when compared to similar sized vessels made from stainless steel, for example. These materials also enable greater flexibility in the design of the drying chamber, allowing the formation of complex shapes, which are much more difficult and expensive to manufacture from stainless steel.

In some aspects, the tank comprises a stirring device (e.g., propeller or stir bar).

In some aspects, the tank is arranged to receive the liquid solvent (e.g., water), the carrier, and the anaerobic cells and/or M. elsdenii cells.

The use of non-metallic, non-conducting dielectric materials to form the drying chamber (such as the engineered plastic composite materials), permits the use of one or more electric fields within the drying chamber itself, to urge the particles/droplets into desired trajectories and/or to urge such particles/droplets from the inlet end toward the outlet end of the drying chamber. Unlike metallic, conductive vessels, it is virtually impossible to develop an electric field inside because all charge accumulates on the surface of the vessel.

In some aspects, the drying chamber may include a first electrode located at or near the inlet end thereof, and a second electrode located at or near the outlet end of the drying chamber. The application of a source of voltage potential between the first and second electrodes induces an electric field within the drying chamber sufficient to urge the particles/droplets into desired trajectories as they dry within the chamber. In some aspects, the desirable trajectory is to cause the lines of the electric field to extend generally parallel to the walls of the drying chamber, even where such walls taper toward the outlet end. To achieve such trajectory, the second electrode would have to be of a relatively small diameter as compared to the first electrode. If the first and second electrodes are of generally the same diameter, then the lines of the electric field would extend generally parallel to the walls of the drying chamber, and then pass through the tapered walls at the outlet end. Other particle trajectories may be achieved based on number, location, size, and shape of the electrodes. In some aspects, the first and second electrodes may be disposed external to the drying chamber, yet induce an electric field within the internal volume of the drying chamber by virtue of the formation thereof from non-electrically conductive material.

In some aspects, the dried particle morphology benefits from a no-heat process in that the particles do not experience an abrupt rise in temperature as they enter the drying chamber and the particles do not exhibit cracks on the surface, volcano structures on the surface, or hollow regions within the particles.

Generally, the no-heat process demonstrates higher levels of preservation of starting active organisms (e.g., anaerobic cells and/or M. elsdenii). In some aspects, the no-heat process resulted in more stable and viable anaerobic cells and/or M. elsdenii than a heated spray drying process. In some aspects, the appearance of non-viable anaerobic cells and/or M. elsdenii was greatly diminished in the no-heat samples, resulting in longer projected shelf-life of the powder comprising anaerobic cells and/or M. elsdenii.

In some aspects, the resultant dried powder includes a plurality of dried particles, which individually contain an amount of final active organism (e.g., anaerobic cells and/or M. elsdenii) encapsulated within a carrier resulting from drying the slurry, which contained an initial active organism, a liquid solvent and the carrier. In some aspects, the initial active organism includes one or more constituent components, at least one of which is among one or more principle molecular types from which at least one of a bacteria, probiotic, etc. is obtained. In some aspects, the bacteria is M. elsdenii as disclosed herein. In some aspects, the bacteria is M. elsdenii NCIMB 41125.

In some aspects, the carrier is a modified starch or carbohydrate. In some aspects, the carrier is selected from the group consisting of a sucrose, a maltodextrin, a maltose, a hydrophobic starch, a sugar, a sugar alcohol, a sugar derivative, or a nitrogen source (for example, but not limited to, ammonium salts and derivatives, yeast extract, skim milk, or corn steep liquor), and a mixture thereof. In some aspects, the carrier is selected from the group consisting of a sucrose, a maltodextrin, a maltose, a polysaccharide, an encapsulating polymer, a hydrophobic starch, a sugar, a sugar alcohol, a sugar derivative, or a nitrogen source (for example, but not limited to, ammonium salts and derivatives, yeast extract, skim milk, or corn steep liquor), and a mixture thereof. In some aspects, the encapsulating polymer is alginate. In some aspects, the carrier has a concentration of 1-50% weight of the entire slurry and/or dried powder. In some aspects, the carrier has a concentration of 1-45% weight of the entire slurry and/or dried powder. In some aspects, the carrier has a concentration of 1-40% weight of the entire slurry and/or dried powder. In some aspects, the carrier has a concentration of 1-35% weight of the entire slurry and/or dried powder. In some aspects, the carrier has a concentration of 1-30% weight of the entire slurry and/or dried powder. In some aspects, the carrier has a concentration of 1-25% weight of the entire slurry and/or dried powder. In some aspects, the carrier has a concentration of 2-20% weight of the entire slurry and/or dried powder. In some aspects, the carrier has a concentration of 1-20% weight of the entire slurry and/or dried powder. In some aspects, the carrier has a concentration of 2-19% weight of the entire slurry and/or dried powder. In some aspects, the carrier has a concentration of 3-18% weight of the entire slurry and/or dried powder. In some aspects, the carrier has a concentration of 4-17% weight of the entire slurry and/or dried powder. In some aspects, the carrier has a concentration of 5-16% weight of the entire slurry and/or dried powder. In some aspects, the carrier has a concentration of 6-15% weight of the entire slurry and/or dried powder. In some aspects, the carrier has a concentration of 7-14% weight of the entire slurry and/or dried powder. In some aspects, the carrier has a concentration of 8-13% weight of the entire slurry and/or dried powder. In some aspects, the carrier has a concentration of 9-12% weight of the entire slurry and/or dried powder. In some aspects, the carrier has a concentration of 10-11% weight of the entire slurry and/or dried powder.

In some aspects, the carrier has a concentration of about 1%, about 2%, about 3%, about 4%, about 5%, about 6%, about 7%, about 8%, about 9%, about 10%, about 11%, about 12%, about 13%, about 14%, about 15%, about 20%, about 25%, about 30%, about 35%, about 40%, about 45%, or about 50% weight of the entire slurry and/or dried powder.

In some aspects, the moisture content is less than 1%, about 2%, about 3%, about 4%, about 5%, about 6%, about 7%, about 8%, about 9%, about 10%, about 11%, about 12%, about 13%, about 14%, about 15%, about 20%, about 25%, about 30%, about 35%, about 40%, about 45%, or about 50% weight of the dried powder. In some aspects, the moisture content is about 1-30% weight of the dried powder. In some aspects, the moisture content is about 3-25% weight of the dried powder. In some aspects, the moisture content is about 5-20% weight of the dried powder. In some aspects, the moisture content is about 10-20% weight of the dried powder.

In some aspects, the final active organism includes one or more of the constituent components corresponding with those of the initial active organism as modified by the drying of the slurry. A weight percentage of at least one of the one or more principle molecular types in the final active organism is essentially the same (e.g., within about 5%, 4%, 3%, or 1% of a) weight percentage of the corresponding principle molecular types in the initial active organism.

The use of high solids content emulsions (with solids concentrations of at least 40% by weight, based on total weight of the emulsion, and preferably at least 50% by weight on the same total weight basis) in the low temperature, e.g., no heat, spray drying process of the present disclosure provides a host of desirable attributes to the final powders produced by the process, such as: (1) high particle density, with density greater than that of water (i.e., >1 g/cc), so that the particles readily sink into aqueous solution and become rapidly dissolved or suspended, (2) greater resistance to oxidation imparted by the higher solids content, (3) substantial energy efficiency from the use of high solids content slurries/emulsions coupled with low temperatures, since about half as much water is evaporated as compared to traditional spray drying processes, and (4) superior retention of high value active organisms such as M. elsdenii, as another substantial economic advantage brought about by low temperature drying and high solids content slurries/emulsions.

However, conventional high temperature drying processes cause a significant loss of viability of anaerobic cells and/or M. elsdenii, resulting in powders with less viable anaerobic cells and/or M. elsdenii. Generally, the powders resulting from high temperature conventional spray drying processes are of small average diameter, e.g., on the order of 60-100 micrometers, are not fully dense, and are difficult to dissolve in aqueous solutions. Powders produced by the low temperature spray drying process of the present disclosure, by contrast, have large average diameters, such as on the order of 125-250 micrometers, are fully dense, and readily go into solution.

Electrospray drying can be a continuous process. In some aspects, the slurry is processed at a rate of between about 0.1 L/hour and about 10,000 L/hour. In some aspects, the slurry is processed at a rate of between about 0.25 L/hour and about 10,000 L/hour. In some aspects, the slurry is processed at a rate of between about 1 L/hour and about 10,000 L/hour. In some aspects, the slurry is processed at a rate of between about 10 L/hour and about 5,000 L/hour. In some aspects, the slurry is processed at a rate of between about 50 L/hour and about 1,000 L/hour. In some aspects, the slurry is processed at a rate of between about 100 L/hour and about 900 L/hour. In some aspects, the slurry is processed at a rate of between about 200 L/hour and about 800 L/hour. In some aspects, the slurry is processed at a rate of between about 300 L/hour and about 700 L/hour. In some aspects, the slurry is processed at a rate of between about 400 L/hour and about 600 L/hour. In some aspects, the slurry is processed at a rate of between about 500 L/hour and about 600 L/hour.

Spray dried powders of the present disclosure thus can be produced in which the final active organism includes one or more of the constituent components corresponding to those of the initial active organism as modified by the spray drying process, wherein the spray dried powder or fiber composition has at least one of the characteristics of (i) a weight percentage of at least one of the one or more principle molecular types (e.g., anaerobic cells and/or M. elsdenii) in the final organism which is within about 15% of a weight percentage of the corresponding principle molecular types in the initial active organism, and which may for example be within about 12%, 10%, 8%, 7%, 6%, 5%, 4%, 3%, 2% or 1% of the weight percentage of the corresponding principle molecular types in the initial active organism.

In some aspects, about 1×10³ to 1×10¹³ CFU/g of electrospray dried anaerobic cells and/or M. elsdenii cells are produced by a method disclosed herein. In some aspects, about 1×10³ to 1×10¹³ CFU/g of anaerobic cells and/or M. elsdenii cells are viable after electrospray drying.

In some aspects, the amount of electrospray dried anaerobic cells and/or M. elsdenii cells produced by a method disclosed herein and/or the amount of anaerobic cells and/or M. elsdenii cells that are viable after electrospray drying is about 1×10³ CFU/g to about 1×10¹³ CFU/g about 1×10³ CFU/g to about 1×10¹² CFU/g, about 1×10³ CFU/g to about 1×10¹¹ CFU/g, about 1×10³ CFU/g to about 1×10¹⁰ CFU/g, about 1×10³ CFU/g to about 1×10⁹ CFU/g, about 1×10³ CFU/g to about 1×10⁸ CFU/g, about 1×10³ CFU/g to about 1×10⁷ CFU/g, about 1×10³ CFU/g to about 1×10⁶ CFU/g, about 1×10⁴ CFU/g to about 1×10¹³ CFU/g, about 1×10⁵ CFU/g to about 1×10¹³ CFU/g, about 1×10⁶ CFU/g to about 1×10¹³ CFU/g, about 1×10⁷ CFU/g to about 1×10¹³ CFU/g, about 1×10⁸ CFU/g to about 1×10¹³ CFU/g, about 1×10⁹ CFU/g to about 1×10¹³ CFU/g, about 1×10¹⁰ CFU/g to about 1×10¹³ CFU/g, about 1×10³ CFU/g to about 1×10⁵ CFU/g, about 1×10⁴ CFU/g to about 1×10⁶ CFU/g, about 1×10⁵ CFU/g to about 1×10⁷ CFU/g, about 1×10⁶ CFU/g to about 1×10⁸ CFU/g, about 1×10⁷ CFU/g to about 1×10⁹ CFU/g, about 1×10⁸ CFU/g to about 1×10¹⁰ CFU/g, about 1×10⁹ CFU/g to about 1×10¹¹ CFU/g, or about 1×10¹⁰ CFU/g to about 1×10¹³ CFU/g.

In some aspects, the electrospray dried anaerobic cells and/or M. elsdenii cells are viable for about 14 days to about 24 months at about −80° C., about −20° C., about 4° C., about 25° C., or combinations thereof. In some aspects, the electrospray dried anaerobic cells and/or M. elsdenii cells are viable for at least 14 days, at least 1 month, at least 6 months, at least 8 months, at least 10 months, at least 12 months, at least 15 months, at least 18 months, or at least 24 months at about −80° C., about −20° C., about 4° C., about 25° C., or combinations thereof.

In some aspects, the electrospray dried anaerobic cells and/or M. elsdenii cells are viable for at least 75 days at about −20° C., about 4° C., about 30° C., or combinations thereof. In some aspects, the electrospray dried anaerobic cells and/or M. elsdenii cells are viable for at least 6 months at about −20° C. In some aspects, the electrospray dried anaerobic cells and/or M. elsdenii cells are viable for at least 28 days at about 37° C.

In some aspects, the electrospray dried anaerobic cells and/or M. elsdenii cells are viable for 14 days, 1 month, 6 months, 8 months, 10 months, 12 months, 15 months, 18 months, or 24 months at about −80° C., about −20° C., about 4° C., about 25° C., or combinations thereof.

In some aspects, the electrospray dried anaerobic cells and/or M. elsdenii cells are viable for 75 days at about −20° C., about 4° C., about 30° C., or combinations thereof. In some aspects, the electrospray dried anaerobic cells and/or M. elsdenii cells are viable for 6 months at about −20° C. In some aspects, the electrospray dried anaerobic cells and/or M. elsdenii cells are viable for 28 days at about 37° C.

In some aspects, about 1×10³ CFU/g to about 1×10¹³ CFU/g about 1×10³ CFU/g to about 1×10¹² CFU/g, about 1×10³ CFU/mL to about 1×10¹¹ CFU/g, about 1×10³ CFU/g to about 1×10¹⁰ CFU/g, about 1×10³ CFU/g to about 1×10⁹ CFU/g, about 1×10³ CFU/g to about 1×10⁸ CFU/g, about 1×10³ CFU/g to about 1×10⁷ CFU/g, about 1×10³ CFU/g to about 1×10⁶ CFU/g, about 1×10⁴ CFU/g to about 1×10¹³ CFU/g, about 1×10⁵ CFU/g to about 1×10¹³ CFU/g, about 1×10⁶ CFU/g to about 1×10¹³ CFU/g, about 1×10⁷ CFU/g to about 1×10¹³ CFU/g, about 1×10⁸ CFU/g to about 1×10¹³ CFU/g, about 1×10⁹ CFU/g to about 1×10¹³ CFU/g, about 1×10¹⁰ CFU/g to about 1×10¹³ CFU/g, about 1×10³ CFU/g to about 1×10⁵ CFU/g, about 1×10⁴ CFU/g to about 1×10⁶ CFU/g, about 1×10⁵ CFU/g to about 1×10⁷ CFU/g, about 1×10⁶ CFU/g to about 1×10⁸ CFU/g, about 1×10⁷ CFU/g to about 1×10⁹ CFU/g, about 1×10⁸ CFU/g to about 1×10¹⁰ CFU/g, about 1×10⁹ CFU/g to about 1×10¹¹ CFU/g, or about 1×10¹⁰ CFU/g to about 1×10¹³ CFU/g of electrospray dried anaerobic cells and/or M. elsdenii cells are viable after storage at a temperature of about −80° C., about −20° C., about 4° C., or combinations thereof for at least 14 days, at least 1 month, at least 6 months, at least 8 months, at least 10 months, at least 12 months, at least 15 months, at least 18 months, or at least 24 months.

In some aspects, about 1×10³ CFU/g to about 1×10¹³ CFU/g about 1×10³ CFU/g to about 1×10¹² CFU/g, about 1×10³ CFU/g to about 1×10¹¹ CFU/g, about 1×10³ CFU/g to about 1×10¹⁰ CFU/g, about 1×10³ CFU/g to about 1×10⁹ CFU/g, about 1×10³ CFU/g to about 1×10⁸ CFU/g, about 1×10³ CFU/g to about 1×10⁷ CFU/g, about 1×10³ CFU/g to about 1×10⁶ CFU/g, about 1×10⁴ CFU/g to about 1×10¹³ CFU/g, about 1×10⁵ CFU/g to about 1×10¹³ CFU/g, about 1×10⁶ CFU/g to about 1×10¹³ CFU/g, about 1×10⁷ CFU/g to about 1×10¹³ CFU/g, about 1×10⁸ CFU/g to about 1×10¹³ CFU/g, about 1×10⁹ CFU/g to about 1×10¹³ CFU/g, about 1×10¹⁰ CFU/g to about 1×10¹³ CFU/g, about 1×10³ CFU/g to about 1×10⁵ CFU/g, about 1×10⁴ CFU/g to about 1×10⁶ CFU/g, about 1×10⁵ CFU/g to about 1×10⁷ CFU/g, about 1×10⁶ CFU/g to about 1×10⁸ CFU/g, about 1×10⁷ CFU/g to about 1×10⁹ CFU/g, about 1×10⁸ CFU/g to about 1×10¹⁰ CFU/g, about 1×10⁹ CFU/g to about 1×10¹¹ CFU/g, or about 1×10¹⁰ CFU/g to about 1×10¹³ CFU/g of electrospray dried anaerobic cells and/or M. elsdenii cells are viable after storage at a temperature of about 25° C. for at least 14 days, at least 1 month, at least 6 months, at least 8 months, at least 10 months, at least 12 months, at least 15 months, at least 18 months, or at least 24 months.

### Feed Additives, Compositions, and Kits

In some aspects, a feed additive comprises electrospray dried anaerobic cells and/or M. elsdenii cells as disclosed herein. In some aspects, the feed additive comprises freeze-dried anaerobic cells and/or M. elsdenii cells produced by a method disclosed herein.

In some aspects, the feed additive is a solid (i.e., “a solid feed additive”) or a liquid (i.e., “a liquid feed additive”). In some aspects, the feed additive is a semi-solid or a gel (i.e., “a semi-solid or a gel feed additive”). A gel feed additive can contain an oxygen scavenger (e.g., ascorbic acid).

In some aspects, a solid feed additive is a powder (e.g., a flowable powder), granule (i.e., a granulate), particle (i.e., particulate), pellet, cake, water soluble concentrate, paste, bolus, tablet, dust, a component thereof, or combinations thereof.

In some aspects, a liquid feed additive is a solution (e.g., an aqueous, organic, or aqueous-organic solution), suspension, emulsion, drench, spray, injectable, drink (e.g., a milk replacer), a component thereof, or combinations thereof.

In some aspects, a gel feed additive is an organogel. In some aspects, the gel feed additive is an oral gel (i.e., a gel for oral administration).

In some aspects, the feed additive is for use as a top dress (i.e., for adding to the surface of a food or mixing with a food (e.g., an animal feed)). In some aspects, the feed additive is for administration as a liquid.

In some aspects, electrospray dried anaerobic cells (e.g., Bifidobacterium cells, such as B. breve, Lactobacillus cells, such as L. plantarum, Bifidobacterium cells, such as B. animalis subsp. lactis, Pediococcus cells, such as P. acidilactici, Lactobacillus cells, such as L. casei, Fibrobacter, such as F. succinogenes, Ruminococcus, such as R. flavefaciens, and Butyrivibrio, such as B. fibrisolvens) and/or M. elsdenii cells as disclosed herein can be used as a liquid feed additive by rehydrating, dissolving, solubilizing, and/or suspending the cells in a liquid.

In some aspects, the feed additive comprises a feed additive carrier (i.e., one or more feed additive carriers).

Examples of suitable feed additive carriers include, but are not limited to, plant materials (i.e., whole plants or plant parts (e.g., seeds, stems, leaves, flowers, and/or roots, for example), including dried or processed plants or plant parts), dried grains (e.g., distillers' dried grains), alfalfa, corn meal, citrus meal, fermentation residues, ground oyster shells, attapulgus clay, wheat shorts, molasses solubles, corn cob meal, edible vegetable substances, toasted dehulled soya flour, soybean mill feed, antibiotic mycelis, vermiculite, soya grits, whey, maltodextrin, sucrose, dextrose, limestone (calcium carbonate), rice hulls, yeast culture, dried starch, sodium silica aluminate, water, salt solutions, alcohol, silicone, waxes, petroleum jelly, vegetable oils, polyethylene glycols, propylene glycol, liposomes, sugars, gelatin, lactose, amylose, magnesium stearate, talc, surfactants, silicic acid, viscous paraffin, perfume oil, fatty acid monoglycerides and diglycerides, petroethral fatty acid esters, hydroxymethyl-cellulose, polyvinylpyrrolidone, and the like, and combinations thereof.

In some aspects, the feed additive comprises an excipient (i.e., one or more excipients) including, but not limited to, microcrystalline cellulose; lactose; sodium citrate; calcium carbonate; dibasic calcium phosphate and glycine; disintegrants such as starch, sodium starch glycollate, croscarmellose sodium and certain complex silicates; granulation binders such as polyvinylpyrrolidone, hydroxypropylmethylcellulose (HPMC), hydroxypropylcellulose (HPC), sucrose, gelatin, and acacia; bulking agents such as maltodextrin; moisture scavengers such as silicon dioxide; oxygen scavengers such as ascorbic acid; and/or lubricating agents such as magnesium stearate, stearic acid, glyceryl behenate, and talc.

In some aspects, the feed additive is a granule comprising: a core comprising the anaerobic cells and/or M. elsdenii cells and/or feed additive, and a coating over the core. In some aspects, the coating is a hydrated barrier salt. The salt coating can provide improved thermo-tolerance, improved storage stability, and protection against other components in the granules that may otherwise have adverse effect (e.g., on stability) of the M. elsdenii cells and/or feed additive.

In some aspects, the electrospray dried anaerobic cells and/or M. elsdenii cells are admixed with a dry formulation of additives including, but not limited to, growth substrates, enzymes, sugars, carbohydrates, extracts, and growth promoting micro-ingredients. The sugars can include, but are not limited to, lactose, maltose, dextrose, maltodextrin, sucrose, glucose, fructose, mannose, tagatose, sorbose, raffinose, amylose, starch, and galactose. The sugars can range from 50-95%, either individually or in combination. The extracts can include, but are not limited to, yeast or dried yeast fermentation solubles ranging from 5-50%. In some aspects, the extracts include condensed fermented corn extractives or corn steep liquor. The growth substrates can include, but are not limited to, trypticase, ranging from 5-25%; sodium lactate, ranging from 5-30%; and Tween 80, ranging from 1-5%. The carbohydrates can include, but are not limited to, mannitol, sorbitol, adonitol and arabitol. The carbohydrates can range from 5-50% individually or in combination. The micro-ingredients can include, but are not limited to, calcium carbonate, ranging from 0.5-5.0%; calcium chloride, ranging from 0.5-5.0%; dipotassium phosphate, ranging from 0.5-5.0%; calcium phosphate, ranging from 0.5-5.0%; manganese proteinate, ranging from 0.25-1.00%; and manganese, ranging from 0.25-1.00%.

In some aspects, the anaerobic cells and/or M. elsdenii feed additive is prepared by mixing (e.g., with a mixer) anaerobic cells and/or M. elsdenii cells, including a culture comprising the cells and/or electrospray dried cells, with any additional components of the feed additive (e.g., a carrier and/or an excipient). In some aspects, the components are mixed to obtain a uniform mixture.

In some aspects, the feed additive is a top-dress animal feed additive comprising anaerobic cells and/or M. elsdenii cells as disclosed herein (e.g., electrospray dried cells) and a carrier. In some aspects, the carrier is selected from the group consisting of: whey, maltodextrin, sucrose, dextrose, limestone (i.e., calcium carbonate), rice hulls, yeast culture, dried starch, and sodium silica aluminate, milk, water, and combinations thereof.

In some aspects, the animal feed additive is a drench, spray, or supplement of a milk replacer comprising anaerobic cells and/or M. elsdenii cells as disclosed herein (e.g., electrospray dried cells) and a water soluble carrier. In some aspects, the carrier is selected from the group consisting of: whey, maltodextrin, sucrose, dextrose, dried starch, sodium silica aluminate, milk, water, and combinations thereof.

In some aspects, the present invention is directed to a food (e.g., an animal feed) comprising anaerobic cells and/or M. elsdenii cells (e.g., electrospray dried cells as disclosed herein, e.g., electrospray dried cells produced by a method as disclosed herein) and/or a feed additive as disclosed herein. A food product is any food for animal consumption (i.e., non-human animals or humans), and includes both solid and liquid compositions. Foods include, but are not limited to, common foods; liquid products, including waters, milks, beverages, therapeutic drinks, and nutritional drinks; functional foods; supplements; nutraceuticals; infant (i.e., including non-human and human infants) formulas, including formulas for pre-mature infants; foods for pregnant or nursing animals; foods for adult animals; and geriatric foods. In some aspects, the food includes a liquid (e.g., a drink, e.g., water, milk, or a milk replacer) comprising the feed additive.

In some aspects, the present disclosure is directed to a composition comprising anaerobic cells and/or M. elsdenii cells (e.g., electrospray dried cells) and/or a feed additive as disclosed herein. In some aspects, the composition comprises electrospray dried anaerobic cells and/or M. elsdenii cells produced by a method disclosed herein.

A composition of the disclosure can include one or more excipients. In some aspects, the excipient can be, but is not limited to, an alkaline agent, a stabilizer, an antioxidant, an adhesion agent, a separating agent, a coating agent, an exterior phase component, a controlled-release component, a solvent, a surfactant, a humectant, a buffering agent, a filler, an emollient, or combinations thereof. Excipients in addition to those discussed herein can include excipients listed in, though not limited to, Remington: The Science and Practice of Pharmacy, 21^{st} ed. (2005). Inclusion of an excipient in a particular classification herein (e.g., “solvent”) is intended to illustrate rather than limit the role of the excipient. A particular excipient can fall within multiple classifications.

In some aspects, the composition is a pharmaceutical composition (e.g., for treatment of non-human animals or humans). In some aspects, the composition is a medical food (e.g., a veterinary food). A medical food includes a food that is in a composition to be consumed or administered externally under the supervision of a doctor (e.g., a veterinarian) and that is intended for the specific dietary management of a condition, for which distinctive nutritional requirements, based on recognized scientific principles, are established by medical evaluation. In some aspects, the pharmaceutical composition comprises a pharmaceutically acceptable excipient. In some aspects, the term “pharmaceutically acceptable” means approved by a regulatory agency of the Federal or a state government or listed in the U.S. Pharmacopeia or other generally recognized international pharmacopeia for use in animals, and more particularly in humans.

For oral administration of a composition, the anaerobic cells and/or M. elsdenii cells (e.g., electrospray dried cells) or feed additive can be combined with excipients well known in the art. Such carriers can, for example, allow the anaerobic cells and/or M. elsdenii cells or feed additive of the invention to be formulated as tablets, pills, dragees, capsules, liquids, gels, syrups, slurries, suspensions and the like, for oral ingestion by a subject to be treated. In some aspects, the composition is a tablet, pill, caplet, or capsule. Suitable excipients include, but are not limited to, fillers such as sugars, including, but not limited to, lactose, sucrose, mannitol, and sorbitol; cellulose preparations such as, but not limited to, maize starch, wheat starch, rice starch, potato starch, gelatin, gum tragacanth, methyl cellulose, hydroxypropylmethyl cellulose, sodium carboxymethyl cellulose, and polyvinylpyrrolidone (PVP). If desired, disintegrating agents can be added, such as, but not limited to, the cross-linked polyvinylpyrrolidone, agar, or alginic acid or a salt thereof such as sodium alginate. Compositions that can be used orally include, but are not limited to, capsules made of gelatin, as well as soft, sealed capsules made of gelatin and a plasticizer, such as glycerol or sorbitol. In some aspects, the dosage form is a vegetarian dosage form, in which the dosage form is not formed from and does not contain any components from an animal source. In some aspects, the vegetarian dosage form is a vegetarian capsule.

In some aspects, the present invention is directed to kits or packages comprising anaerobic cells and/or M. elsdenii cells, feed additives, foods, and/or compositions as disclosed herein. Kits or packages can include units of a feed additive, food, composition, or combinations thereof (e.g., one or more units). In some aspects, the kit comprises freeze-dried cells produced by a method disclosed herein, a feed additive as disclosed herein, or a capsule as disclosed herein.

Methods of Administering Electrospray Dried Anaerobic Bacterial Cells and/or M. elsdenii to Animals

In some aspects, the present invention is directed to a method of administering electrospray dried M. elsdenii cells to an animal.

In some aspects, the present invention is directed to a method of administering electrospray dried anaerobic bacterial cells to an animal. In some aspects, the present invention is directed to a method of administering electrospray dried Bifidobacterium cells, such as B. breve, Lactobacillus cells, such as L. plantarum, Bifidobacterium cells, such as B. animalis subsp. lactis, Pediococcus cells, such as P. acidilactici, Lactobacillus cells, such as L. casei cells, Fibrobacter, such as F. succinogenes cells, Ruminococcus, such as R. flavefaciens, and Butyrivibrio, such as B. fibrisolvens cells to an animal.

In some aspects, the method comprises administering to the animal electrospray dried M. elsdenii cells, a feed additive, a food, or a composition (e.g., a capsule) as described herein.

The administration can be by any compatible route, including, for example, orally (i.e., an ingestible liquid or solid, an oral drench, a feed additive, a food, a composition, or a capsule), by spraying onto the body (i.e., by mist spraying), and/or injection.

In some aspects, the method comprises administering a solid, a liquid, or a gel comprising the electrospray dried M. elsdenii or anaerobic cells.

In some aspects, the method comprises administering a solid feed additive comprising the electrospray dried M. elsdenii or anaerobic cells. In some aspects, the solid feed additive is a powder (e.g., a flowable powder), granule (i.e., a granulate), particle (i.e., particulate), pellet, cake, water soluble concentrate, paste, bolus, tablet, dust, a component thereof, or combinations thereof.

In some aspects, the method comprises administering a liquid feed additive comprising the electrospray dried M. elsdenii or anaerobic cells. In some aspects, the method comprises administering the electrospray dried M. elsdenii or anaerobic cells in a liquid. In some aspects, the liquid is a solution (e.g., an aqueous, organic, or aqueous-organic solution), suspension, emulsion, drench, spray, injectable, drink (e.g., a milk replacer), a component thereof, or combinations thereof. In some aspects, the liquid is administered orally or by spraying the animal with the liquid.

In some aspects, the method comprises combining the electrospray dried M. elsdenii or anaerobic cells or the feed additive comprising the cells with another animal feed additive to form a supplement or premix for adding to an animal feed. In some aspects, the other feed additive comprises cells other than M. elsdenii.

In some aspects, the electrospray dried M. elsdenii or anaerobic cells can be added to the feed additive as a liquid (e.g., in a broth or broth equivalent, including, e.g., rehydrated electrospray-dried cells), or as a reconstituted cell paste. Dosage forms (e.g. drench of predetermined volume or capsules) can also be formed and, if desired, the electrospray dried M. elsdenii or anaerobic cells can be added directly to the animal feed, as by sprinkling a liquid broth and/or electrospray dried M. elsdenii or anaerobic cells over the feed or mixing into the feed.

In some aspects, the method comprises rehydrating a feed additive (e.g., a powder, granulate, particulate, pellet, cake, electrospray-dried cells, or combinations thereof) to produce a liquid for administration.

In some aspects, the method comprises applying electrospray dried M. elsdenii or anaerobic cells to animal feed through a delivery system that rehydrates a feed additive, including on a batch-to batch basis. For example, an electrospray dried powder can be augured from a polyvinyl hopper into a flushing system, which dilutes the powder and sprays it on the feed to be mixed.

In some aspects, the method comprises applying electrospray dried M. elsdenii or anaerobic cells to animal feed using a volumetric metering device with a storage bin. For example, the electrospray dried M. elsdenii or anaerobic cells (e.g., a powder comprising the cells) can be stored in the storage bin and discharged into a water or aqueous bath just prior to being sprayed onto an animal feed.

In some aspects, the present invention is directed to a method for treating or preventing a condition or disorder associated with lactic acid production in the gastrointestinal tract of an animal, comprising administering to the animal an effective amount of electrospray dried M. elsdenii as disclosed herein, electrospray dried M. elsdenii cells produced by a method as disclosed herein, a feed additive as disclosed herein, or a composition (e.g., a capsule) as disclosed herein.

In some aspects, the present invention is directed to a method for treating or preventing a condition or disorder associated with lactic acid production in the gastrointestinal tract of an animal, comprising administering to the animal an effective amount of electrospray dried anaerobic cells as disclosed herein, electrospray dried anaerobic cells produced by a method as disclosed herein, a feed additive as disclosed herein, or a composition (e.g., a capsule) as disclosed herein.

In some aspects, the condition or disorder is acidosis. In some aspects, the condition or disorder is ruminal acidosis. In some aspects, the condition or disorder is respiratory disease. In some aspects, the condition or disorder is laminitis. In some aspects, the condition or disorder is an infection. In some aspects, the infection is with Salmonella or Campylobacter. In some aspects, the Salmonella is Salmonella enterica and/or Salmonella bongori. In some aspects, the Salmonella serotype is Salmonella Typhimurium and/or Enteritidis. In some aspects, the Campylobacter is Campylobacter jejuni or Campylobacter coli. In some aspects, the condition or disorder is hindgut acidosis.

In some aspects, the condition or disorder is a condition or disorder in which the proportion of butyrate, valerate, and/or propionate are increased.

In some aspects, the present invention is directed to a method for preventing or decreasing the growth of an opportunistic microorganism in the gastrointestinal tract of an animal, comprising administering to the animal an effective amount of electrospray dried M. elsdenii cells as disclosed herein, electrospray dried M. elsdenii cells produced by a method as disclosed herein, a feed additive as disclosed herein, or a composition as disclosed herein.

In some aspects, the present invention is directed to a method for preventing or decreasing the growth of an opportunistic microorganism in the gastrointestinal tract of an animal, comprising administering to the animal an effective amount of electrospray dried anaerobic cells as disclosed herein, electrospray dried anaerobic cells produced by a method as disclosed herein, a feed additive as disclosed herein, or a composition as disclosed herein.

In some aspects, the opportunistic microorganism is pathogenic. In some aspects, the opportunistic microorganism is Salmonella or Campylobacter. In some aspects, the Salmonella is Salmonella enterica and/or Salmonella bongori. In some aspects, the Salmonella serotype is Salmonella Typhimurium and/or Enteritidis. In some aspects, the Campylobacter is Campylobacter jejuni or Campylobacter coli. In some aspects, the opportunistic microorganism is Escherichia coli.

In some aspects, the present invention is directed to a method of improving the bioavailability of plant-derived phosphorous in the diet of an animal, comprising administering to the animal an effective amount of electrospray dried M. elsdenii cells as disclosed herein, electrospray dried M. elsdenii cells produced by a method as disclosed herein, a feed additive as disclosed herein, or a composition as disclosed herein. In some aspects, the electrospray dried M. elsdenii cells comprise a phytase activity. In some aspects, the method reduces environmental phosphorous waste resulting from administration of an animal diet in the absence of M. elsdenii cells.

In some aspects, the present invention is directed to a method of improving the bioavailability of plant-derived phosphorous in the diet of an animal, comprising administering to the animal an effective amount of electrospray dried anaerobic cells as disclosed herein, electrospray dried anaerobic cells produced by a method as disclosed herein, a feed additive as disclosed herein, or a composition as disclosed herein. In some aspects, the electrospray dried anaerobic cells comprise a phytase activity. In some aspects, the method reduces environmental phosphorous waste resulting from administration of an animal diet in the absence of the anaerobic cells.

In some aspects, the present invention is directed to a method of improving growth performance in an animal, comprising administering to the animal an effective amount of electrospray dried M. elsdenii cells as disclosed herein, electrospray dried M. elsdenii cells produced by a method as disclosed herein, a feed additive as disclosed herein, or a composition as disclosed herein.

In some aspects, the present invention is directed to a method of improving growth performance in an animal, comprising administering to the animal an effective amount of electrospray dried anaerobic cells as disclosed herein, electrospray dried anaerobic cells produced by a method as disclosed herein, a feed additive as disclosed herein, or a composition as disclosed herein.

In some aspects, the present invention is directed to a method of increasing pH of the hindgut in an animal, comprising administering to the animal an effective amount of electrospray dried anaerobic cells as disclosed herein, electrospray dried anaerobic cells produced by a method as disclosed herein, a feed additive as disclosed herein, or a composition as disclosed herein.

In some aspects, the improved growth performance in the animal is an improvement in: feed intake, average daily gain, feed conversion ratio, carcass gain, milk production in a milk-producing animal, egg production in poultry, bone mineralization, or combinations thereof.

In some aspects, the present invention is directed to a method of acidifying the lower gastrointestinal tract of an animal, comprising administering to the animal an effective amount of electrospray dried M. elsdenii cells as disclosed herein, electrospray dried M. elsdenii cells produced by a method as disclosed herein, a feed additive as disclosed herein, or a composition as disclosed herein. In some aspects, the lower gastrointestinal tract is the ceca of a poultry animal.

In some aspects, the present invention is directed to a method of acidifying the lower gastrointestinal tract of an animal, comprising administering to the animal an effective amount of electrospray dried anaerobic cells as disclosed herein, electrospray dried anaerobic cells produced by a method as disclosed herein, a feed additive as disclosed herein, or a composition as disclosed herein. In some aspects, the lower gastrointestinal tract is the ceca of a poultry animal.

In some aspects, the electrospray dried M. elsdenii or anaerobic cells as disclosed herein, electrospray dried M. elsdenii or anaerobic cells produced by a method as disclosed herein, a feed additive as disclosed herein, or a composition as disclosed herein is administered prior to, concomitantly with, or after feeding the animal with a food.

In some aspects, the method further comprises mixing electrospray dried M. elsdenii or anaerobic cells as disclosed herein, the electrospray dried M. elsdenii or anaerobic cells produced by a method as disclosed herein, or a solid feed additive as disclosed herein with a liquid prior to administration.

In some aspects, a liquid is administered orally (e.g., an oral drench) or by spraying (e.g., mist spraying) the animal with the liquid.

In some aspects, the method comprises a single administration of electrospray dried M. elsdenii or anaerobic cells as disclosed herein, electrospray dried M. elsdenii or anaerobic cells produced by a method as disclosed herein, a feed additive as disclosed herein, or a composition as disclosed herein.

In some aspects, the method comprises administration of electrospray dried M. elsdenii or anaerobic cells as disclosed herein, electrospray dried M. elsdenii or anaerobic cells produced by a method as disclosed herein, a feed additive as disclosed herein, or a composition as disclosed herein. In some aspects, the administration comprises dusting an animal (e.g., a bird) with the electrospray dried M. elsdenii or anaerobic cells as disclosed herein, electrospray dried M. elsdenii or anaerobic cells produced by a method as disclosed herein, a feed additive as disclosed herein, or a composition as disclosed herein. In some aspects, the electrospray dried M. elsdenii or anaerobic cells as disclosed herein, electrospray dried M. elsdenii or anaerobic cells produced by a method as disclosed herein, a feed additive as disclosed herein, or a composition as disclosed herein, is ingested by the bird via preening.

In some aspects, the method comprises a daily administration of electrospray dried M. elsdenii or anaerobic cells as disclosed herein, electrospray dried M. elsdenii or anaerobic cells produced by a method as disclosed herein, a feed additive as disclosed herein, or a composition as disclosed herein. In some aspects, the administration is at least once daily, at least twice daily, at least three times daily, or more than three times daily. In some aspects, the administration is ad libitum (e.g., self-administration by drinking an available liquid or eating an available food comprising the electrospray dried M. elsdenii or anaerobic cells, the electrospray dried M. elsdenii or anaerobic cells produced by a method as disclosed herein, the feed additive, or the composition).

In some aspects, the method comprises more than one administration on a single day of electrospray dried M. elsdenii or anaerobic cells as disclosed herein, electrospray dried M. elsdenii or anaerobic cells produced by a method as disclosed herein, a feed additive as disclosed herein, or a composition as disclosed herein. In some aspects, the administration is two, three, four, five, six, or more administrations on a single day. In some aspects, the method comprises more than one administration on a single day followed by one or more days without administration. In some aspects, the one or more days without administration is one, two, three, four, five, or six days, one week, two weeks, three weeks, or four weeks, one month, two months, three months, four months, five months, or six months without administration.

In some aspects, the animal is a ruminant. In some aspects, the ruminant can be, but is not limited to, cattle, buffalo, sheep, goats, deer, reindeer, moose, giraffe, yaks, and elk. In some aspects, the ruminant is selected from the group consisting of: cattle, buffalo, sheep, goats, deer, and reindeer.

In some aspects, the animal is a non-ruminant. In some aspects, the non-ruminant can be, but is not limited to, equines, poultry, swine, dogs, and cats. In some aspects, the non-ruminant is selected from the group consisting of: equines, poultry, and swine.

In some aspects, the animal is a zoo animal.

In some aspects, the animal is a poultry animal. In some aspects, the poultry animal is an avian (i.e., bird) that is used as a food animal including, but not limited to, a chicken, goose, duck, quail, turkey, pigeon, emu, or ostrich. In some aspects, the poultry animal is selected from the group consisting of: a chicken, a goose, a duck, a quail, a turkey, or a pigeon. In some aspects, the poultry animal is selected from the group consisting of: a broiler, a broiler breeder, and a layer. In some aspects, the poultry animal is a chicken.

In some aspects, the animal is an equine. In some aspects, the equine is a horse, a pony, a donkey, or a mule.

### EXAMPLES

Reference is now made to the following examples, which together with the above descriptions illustrate some aspects of the invention in a non-limiting fashion.

### Example 1

Laboratory Scale Production of Electrospray Dried M. elsdenii

M. elsdenii NCIMB 41125 cultures were prepared in the laboratory using laboratory scale fermenters. Two 10-liter bioreactors of semi defined media, consisting of two carbon sources, were prepared to grow M. elsdenii NCIMB 41125.

Bioreactors were inoculated at a 1:100 ratio with an overnight culture of M. elsdenii NCIMB 41125 and incubated for 15 hours at 39° C. under anaerobic conditions using a nitrogen blanket.

The resulting culture was cooled to room temperature (approximately 25° C.) and either packaged in polyfoil bags in 900 mL aliquots (1X cell culture or fermentate) and stored at 4° C. until further processing. The stored cell cultures were used for feedstock numbers 1-6, 8, 10, and 11.

Cells prepared from the above culture were placed into centrifugation bottles under anaerobic conditions (900 mL per bottle, 11 bottles total), and centrifuged for 10 minutes at 5,200 rpm at 20° C. After centrifuging, the supernatant was removed to obtain a 100× concentrate of the cells (9 mL per bottle, 11 bottles total). The concentrated cell pellets were resuspended in supernatant to obtain a 10× cell concentration.

An additional bag of culture (approximately 900 mL) was centrifuged under the same conditions. This concentrated cell pellet was resuspended in anaerobic reverse osmosis (RO) water to obtain a 1× cell concentration (9 mL cell concentrate+891 mL RO water) for feedstock number 7.

The cell concentrates (10×) were combined, thoroughly agitated, and packaged under sterile and anaerobic conditions into a polyfoil bag (900 mL per bag). The cell concentrate (1×) was packaged following the same method. Both bags were stored at 4° C. until further processing for electrospray drying.

Stabilizer solutions were added to the cell concentrates under sterile and anaerobic conditions prior to electrospray drying the cells to maximize cell survival. Stabilizer solution is a carrier solubilized in water, which also serves as a protectant to the cells, and the terms are used interchangeably throughout.

The carriers were left overnight in the anaerobic chamber to minimize oxygen content, then packaged into sterile anaerobic borosilicate glass bottles equipped with a tri-port.

Sterile anaerobic reverse osmosis water (“anaerobic RO water”) was prepared in borosilicate glass bottles equipped with a tri-port.

Final stabilizer solutions were prepared by adding RO water to carbohydrate powders in borosilicate glass bottles under anaerobic conditions. The different stabilizer solutions and components are listed in Table 2.

TABLE 2
Components of stabilizer solutions

| | Carbohydrate | Carbohydrate | RO water amount, |
|---|---|---|---|
| Stabilizer Type | added | amount, g | mL (qs to) |
| 1X Sucrose | Sucrose | 62.50 | 100 |
| 2X Sucrose | Sucrose | 125.00 | 175 |
| 1X (Sucrose + Starch) | Sucrose | 62.50 | 250 |
| | Starch | 100.00 | |
| 1X (Maltodextrin + | Maltodextrin | 46.90 | 75 |
| Maltose) | Maltose | 15.63 | |
| 2X (Maltodextrin + | Maltodextrin | 93.75 | 100 |
| Maltose) | Maltose | 31.20 | |

To prepare the different feedstocks (i.e., cell suspensions) for electrospray drying, the cell concentrates (1× or 10×) and the stabilizer solutions were mixed in the ratio indicated in Table 3. All additions were made under a nitrogen blanket to maintain oxygen-free environment.

TABLE 3
Components of feedstocks for electrospray drying

| | Cell | Cell con- | Stabilizer | Total |
|---|---|---|---|---|
| Feedstock # | concentrate | centrate, part | solution, part | weight, g |
| 1 | 1X | 8.91 | 1.09 | 1134 |
| 2 | 1X | 8.87 | 1.13 | 1087 |
| 3 | 1X | 8.92 | 1.08 | 1097 |
| 4 | 1X | 8.27 | 1.73 | 1298 |
| 5 | 1X* | 8.84 | 1.16 | 1034 |
| 6 | 1X | 7.54 | 2.46 | 1347 |
| 7 | 10X | 8.80 | 1.20 | 1020 |
| 8 | 1X | 9.86 | 0.14 | 1028 |
| 9 | 1X | 9.09 | 0.91 | 1127 |

*Cell concentrate resuspended to 1X with sterile anaerobic reverse osmosis water and not supernatant.

The concentration of M. elsdenii NCIMB 41125 cells was assessed in liquid cultures (cell culture, cell concentrate, cell suspensions/feedstock) and in dry samples obtained after electrospray drying.

The liquid samples were obtained (1) at 15 hours of incubation in the 10 L bioreactor (cell culture), (2) after harvesting the cells using centrifugation (10× cell concentrate), and (3) after addition of the stabilizer solution to the cell concentrate (final feedstock). All treatments were sampled in triplicate under sterile and anaerobic conditions, diluted in sterile anaerobic diluent, and plated onto semi-defined lactate agar (SDL agar) in duplicates. Agar plates were then incubated for 48 hours at 39° C., after which colony forming units (CFU) were counted and concentration per milliliter for each sample was calculated.

Dry samples obtained post-electrospray drying were weighed, and 0.32 g of the dry sample was resuspended in 40 mL of sterile anaerobic diluent. Samples were rehydrated for 2 hours at room temperature before being further diluted and plated on semi-defined lactate agar. Plates were processed as described above to determine final cell concentration in dry samples.

Cell recovery post-electrospray drying was calculated by dividing the total amount of M. elsdenii cells in the dry sample by the initial total amount of M. elsdenii cells in the pre-electrospray dried sample. See formula below.

$$CFU Recovery \% = \frac{\begin{matrix}[ CFU / g ] * [ total volume spray dried ( mL ) ] * \\ [ total solids ( g ) / volume ( mL ) ]\end{matrix}}{[ CFU / mL ] * [ total fermentation volume dried ( mL ) ]} \times 100 \%$$

Dry powder of M. elsdenii NCIMB 41125 was prepared using an electrostatic spray dryer. The drying was achieved by using a heated nitrogen gas with a flow rate of 150 Nm³/h flow rate, the regular inlet temperature ranged between 50-100° C.

The feedstock (cell suspension) was maintained at 4° C. through a chilled water stream delivered to a jacketed beaker where the feedstock was maintained and continuously-stirred by an overhead stirrer. A nitrogen blanket was constantly flushed through the headspace of the vessel to maintain oxygen-free environment.

The feedstock (cell suspension) was delivered through a peristatic pump to a two fluid nozzle at the top of the electrostatic spray dryer chamber, where the feedstock was atomized and electrically-charged. The atomization was achieved through heated pressurized nitrogen gas at 350 kPa and 35° C.

The charging was delivered through a voltage generator controlled by a Pulse Width Modulator, to the electrode in contact with the suspension prior to the nozzle. The atomized feedstock was dried concurrently in the chamber and the dried powder was collected at the end of the vertical chamber.

At the end of the electrospray-drying process, the dried powder was collected, stored in a metallized bag or moisture protective container, flushed with nitrogen and stored at 4° C. for long term storage. The results of the process are shown in Table 4.

TABLE 4
Components of electrospray drying and results after electrospray drying

| | | | | | Cell | | Powder | Powder | | |
|---|---|---|---|---|---|---|---|---|---|---|
| | | | Cell | Inlet | culture | Powder | CFU/g | CFU/g | | |
| | Cell | | Suspension | Temp | CFU/mL | CFU/g | Lower | Upper | Powder | % CFU |
| No | Conc | Stabilizer | Matrix | (° C.) | Average | Average | Limit | Limit | Aw | Recovery |
| 1 | 1X | 1X Sucrose | Supernatant | 80 | 9E+08 | 2E+08 | 8E+07 | 3E+08 | 0.13 | 2.6% |
| 2 | 1X | 1X Sucrose | Supernatant | 70 | 9E+08 | 9E+08 | 7E+08 | 1E+09 | 0.147 | 10.6% |
| 3 | 1X | 1X Sucrose | Supernatant | 90 | 9E+08 | 2E+08 | 2E+08 | 3E+08 | 0.209 | 2.6% |
| 4 | 1X | 2X Sucrose | Supernatant | 80 | 9E+08 | 2E+08 | 1E+08 | 4E+08 | 0.246 | 4.8% |
| 5 | 1X | 1X Sucrose in | Anaerobic | 80 | 4E+08 | 3E+08 | 2E+08 | 5E+08 | 0.151 | 6.1% |
| | | RO water | RO water | | | | | | | |
| 6 | 1X | 1X Sucrose + | Supernatant | 70 | 9E+08 | 6E+07 | 1E+07 | 2E+08 | 0.131 | 1.2% |
| | | 1X HICAP | | | | | | | | |
| | | starch | | | | | | | | |
| 7 | 10X | 1X Sucrose | Supernatant | 80 | 3E+09 | 6E+08 | NA | NA | 0.22 | 2.0% |
| 8 | 1X | 1X | Supernatant | 70 | 9E+08 | 1E+08 | 4E+07 | 2E+08 | 0.217 | 1.2% |
| | | Maltodextrin + | | | | | | | | |
| | | Maltose (3:1) | | | | | | | | |
| 9 | 1X | 2X | Supernatant | 70 | 9E+08 | 4E+07 | 6E+06 | 1E+08 | 0.22 | 0.8% |
| | | Maltodextrin + | | | | | | | | |
| | | Maltose (3:1) | | | | | | | | |

Among the tested inlet temperatures for the electrospray drying process using the same carrier/stabilizer solution (1× sucrose), 70° C. yielded the highest CFU recovery at 10.6%. Increased inlet temperatures of 80° C. and 90° C. decreased the yield to 2.6% each. Additionally, electrospray drying at the higher temperatures (i.e., 80° C. and 90° C.) did not significantly reduce the moisture level in the resulting powder.

Among tested carriers/stabilizer solutions (sucrose, maltodextrin+maltose, hydrophobic starch), sucrose yielded the highest CFU, and 2× sucrose also further improved the % CFU recovery for 80° C. Performing electrospray drying with an inlet temperature of 70° C. with sucrose and a hydrophobic starch reduced % CFU recovery.

Cell concentration (1× or 10×) did not appear to impact the % CFU recovery. However, the 10× cell sample, while being concentrated by 10-fold, experienced a decrease in CFU during storage prior to electrospray drying, which resulted in the lower-than-expected CFU concentration.

### Example 2

Commercial Scale Production of Electrospray Dried M. elsdenii NCIMB 41125

A 10-liter bioreactor of semi-defined media, consisting of two carbon sources, was used to prepare the inoculum. The bioreactor was inoculated at a 1:100 ratio with an overnight culture of M. elsdenii NCIMB 41125 and incubated for 14 hours at 39° C. The resulting culture was cooled to room temperature and packaged into two 5 L polyfoil bags.

One production tank was used to prepare 2,100 L of semi-defined media, consisting of two carbon sources, under sterile and anaerobic conditions to grow M. elsdenii NCIMB 41125. The production tank was inoculated with one of the 5 L cultures of M. elsdenii NCIMB 41125 prepared above. After inoculation, the production tank was incubated at 39° C. for 14 hours followed by cooling to room temperature. A 4 L aliquot of the cell culture was obtained using sterile anaerobic techniques to assess glucose level, OD, pH, contaminant levels (aerobic growth, yeast, and mold), M. elsdenii concentration, and specific gravity.

The contents of production tank (cell culture) were harvested using tangential flow filtration (TFF). Briefly, the cell culture was circulated through the TFF system and the supernatant was discarded until only 21 L of retentate remained in the tank. At this time, retentate was rinsed and resuspended using 63 L of RO water (prepared under anaerobic and sterile conditions) to reach a final retentate concentration of 25×. Final retentate (84 L) was packaged under sterile and anaerobic conditions into 5 L polyfoil bags, stored at 4° C. until electrospray drying.

To prepare the stabilizer solution (carrier), sucrose powder was placed in an anaerobic chamber overnight to minimize oxygen content, then packaged into sterile and anaerobic borosilicate bottles equipped with a tri-port. Sterile and anaerobic RO water was prepared in borosilicate bottles equipped with a tri-port. The stabilizer solution (sucrose solution) was prepared by adding RO water to the sucrose powder via the tri-port under anaerobic conditions as indicated in Table 5.

TABLE 5
Components of stabilizer solution

| | Carbohydrate | Carbohydrate | RO water amount, |
|---|---|---|---|
| Stabilizer type | added | amount, g | mL (qs to) |
| 1X Sucrose | Sucrose | 125 | 200 |

The different feedstocks (cell suspensions) that were electrospray dried were prepared by mixing the 25× cell concentrate (retentate) obtained after TFF and the stabilizer solution. All steps were performed under a nitrogen blanket to maintain an oxygen-free environment at all times. The final sucrose concentration and cell concentration in feedstock were the same for each sample (i.e., 62.5 mg/mL and about 22.5× compared to the initial culture, respectively).

The concentration of M. elsdenii NCIMB 41125 cells was assessed in liquid cultures (cell culture, cell concentrate/retentate, cell suspensions/feedstock) and in dry samples obtained after electrospray drying.

The liquid samples were obtained (1) after 17 hours of incubation in the production tank (cell culture), (2) after harvesting the cells using the TFF system (25× cell concentrate/retentate), and (3) after addition of the stabilizer solution (sucrose solution) to the cell concentrate (final feedstock, 22.5×). All treatments were sampled in triplicate under sterile and anaerobic conditions, diluted in sterile anaerobic diluent, and plated onto semi-defined lactate agar (SDL agar) in duplicates. Agar plates were then incubated for 48 hours at 39° C., after which colony forming units (CFU) were counted and concentration per milliliter for the sample was calculated.

Dry samples obtained post-electrospray drying were weighed, and 0.3 g of the dry sample was resuspended in 60 mL of sterile anaerobic diluent or sterile anaerobic diluent supplemented with 10 g/L of yeast and 5 g/L of soy peptone. Samples rehydrated for 2 hours at room temperature before being further diluted in anaerobic diluent (supplemented or not) and plated into SDL agar or enriched SDL agar with 1 mL of supplemented anaerobic diluent overnight. Plates were processed as above to determine final cell concentration in dry samples.

Cell recovery post-electrospray drying was calculated by dividing the total amount of M. elsdenii in the dry sample by the initial total amount of M. elsdenii in the final feedstock sample (pre-electrospray drying).

$$CFU Recovery \% = \frac{\begin{matrix}[ CFU / g ] * [ total volume spray dried ( mL ) ] * \\ [ total solids ( g ) / volume ( mL ) ]\end{matrix}}{[ CFU / mL ] * [ total volume of fermentate spray dried ( mL ) ]} \times 100 \%$$

Dry powder of M. elsdenii NCIMB 41125 was prepared using an electrostatic spray dryer. The drying was achieved through a heated nitrogen gas with a flow rate of 150 Nm³/h flow rate, the regular inlet temperature ranged between 50-100° C.

The feedstock (cell suspension) was kept at 4° C. through a chilled water stream delivered to a jacketed beaker where the feedstock was maintained and continuously-stirred by an overhead stirrer. A nitrogen blanket was constantly flushed through the headspace of the vessel to maintain an oxygen-free environment.

The feedstock (cell suspension) was delivered through a peristatic pump to a two fluid nozzle at the top of the electrostatic spray dryer chamber, where the feedstock was atomized and electrically-charged. The atomization was achieved through heated pressurized nitrogen gas at 350 kPa and 35° C.

The charging was delivered through a voltage generator controlled by a Pulse Width Modulator, to the electrode in contact with the suspension prior to the nozzle. The atomized feedstock was dried concurrently in the chamber and the dried powder was collected at the end of the vertical chamber.

At the end of the electrospray-drying process, the dried powder was collected, stored in a metallized bag or moisture protective container, flushed with nitrogen and stored at 4° C. for long term storage. The results of the process are shown in Table 6.

TABLE 6
Components of electrospray drying and results after electrospray drying

| | | | | 24X | | | | | |
|---|---|---|---|---|---|---|---|---|---|
| | | | | Concentrate | | Powder | Powder | | |
| | Final | | Inlet | Fermentate | Powder | CFU/g | CFU/g | | |
| | Feedstock | | Temp | CFU/mL | CFU/g | Lower | Upper | Powder | % CFU |
| No | cell conc | Stabilizer | (° C.) | Average | Average | Limit | Limit | Aw | Recovery |
| 1 | 21.6X | 1X Sucrose | 70 | 6.5E+10 | 8.1E10 | 6.0E10 | 1.1E11 | 0.202 | 13% |
| 2 | 21.6X | 1X Sucrose | 65 | 6.5E+10 | 4.5E10 | 2.1E10 | 6.6E10 | 0.201 | 9% |
| 3* | 21.6X | 1X Sucrose | 62.5 | 6.5E+10 | 2.3E10 | 5.5E9 | 3.7E10 | 0.05 | 4% |

The results demonstrate that electrospraying concentrated feedstock of 21.6× with 1× sucrose solution at 70° C. yielded comparable % CFU recovery as in Example 1. The results indicate that a more concentrated M. elsdenii feedstock can be electrospray dried without an impact on % CFU recovery. However, decreasing the temperature to 65° C. and 62.5° C., respectively, did not improve recovery.

### Example 3

Conventional Spray Drying Will Result in a Reduction Recovery of Viable M. elsdenii Cells Compared to Electrospray Drying

M. elsdenii will be cultured and processed for conventional spray drying as described in Example 2.

A conventional spray dryer will be used to produce a dried M. elsdenii powder using sucrose, maltose, maltodextrin, sugar alcohols or starch mixture as the stabilizers with the following spray conditions (Table 7).

TABLE 7
Characteristics of electrospray drying process

| | Matrix/Stabilizer | Spray Inlet | Spray Outlet | |
|---|---|---|---|---|
| Cell | Concentration (final | Temperature | Temperature | |
| Concentration | concentration) | (° C.) | (° C.) | Drying gas |
| 1X | 2-20% of sucrose | 110-150 | 70-110 | Air or |
| | 2-20% of maltose + maltodextrin | | | Nitrogen |
| | 2-20% of sugar alcohols | | | (anaerobic) |

It is expected that the conventional spray drying process will produce a sufficiently dried powder (Aw≤0.3) with an inlet temperature greater than 120° C. while spray drying with nitrogen anaerobically. The % CFU recovery is expected to be <1%. Performing the same process, but with air as the drying gas is expected to yield <0.1% % CFU recovery. Comparatively, electrospray drying is expected to yield 10-100-fold higher % CFU recovery.

### Example 4

Electrospray Drying Results in an Increase of Viable M. elsdenii and Dried Powder Production Compared to Freeze-Drying

M. elsdenii NCIMB 41125 cells were grown in semi-defined media, consisting of two carbon sources, under sterile and anaerobic conditions in a 500-liter tank as described in Example 13 of WO 2018/144653 A1, which is incorporated by reference herein.

At the end of the cell growth phase, the cell culture was concentrated using a tangential flow filtration system (TFF) in order to remove 90% of the liquid (permeate) and collect a 10× retentate.

Retentate was then mixed with 8% trehalose/15% skim Milk (T/SM) or 8% maltodextrin/15% skim milk (M/SM). Retentates mixed with the appropriate cryoprotectant were sampled to determine pre-freeze-drying M. elsdenii concentration (i.e., viability count). Mixtures were distributed into vials (4 mL/vial) and frozen at −80° C. or in liquid nitrogen, and freeze-dried using the rapid cycle.

Once the freeze-drying process was complete, survival of the bacteria was determined by resuspending the lyophilized product in the anaerobic chamber with anaerobic diluent, allowing it to rehydrate for 40 minutes at room temperature, and then plating onto SDL20 agar.

Retentate samples were tested for cell survival during storage at 4° C. or 25° C. in aerobic or anaerobic conditions for 0, 2, 4, 8, 12, 16, 20, and 24 weeks using spread plating technique. Briefly, stored products were sampled, serially diluted, and plated onto a SDL agar plate. The experiment was repeated on 3 different days and all treatments were performed in triplicate.

Cell recovery was calculated by dividing the concentration of M. elsdenii cells recovered post-freeze-drying by the initial concentration of M. elsdenii cells measured in the corresponding retentate mixed with cryoprotectants or not.

The results demonstrate that recovery of post-freeze-drying M. elsdenii cells containing no cryoprotectant (control) was close to zero (Table 8). Samples flash frozen in liquid nitrogen had higher recovery than their counterpart frozen at −80° C., regardless of the cryoprotectant used. Best recoveries were observed with M/SM and T/SM frozen in liquid nitrogen, 5.82% and 3.96%, respectively. Those recoveries are still lower than the recoveries observed from the electrospray dried runs with inlet temperature of 70° C. in Example 1 and 2.

TABLE 8
Recovery of M. elsdenii after freeze-drying using
various cryoprotectants and freezing temperatures

| Cryoprotectant | Freezing | % Recovery |
|---|---|---|
| Control | −80° C. | 0.00% |
| | Liquid nitrogen | 0.00% |
| M/SM | −80° C. | 1.18% |
| | Liquid nitrogen | 5.82% |
| T/SM | −80° C. | 0.30% |
| | Liquid nitrogen | 3.96% |

Additionally, electrospray drying allows for greater production of dried powder containing M. elsdenii cells than freeze-drying.

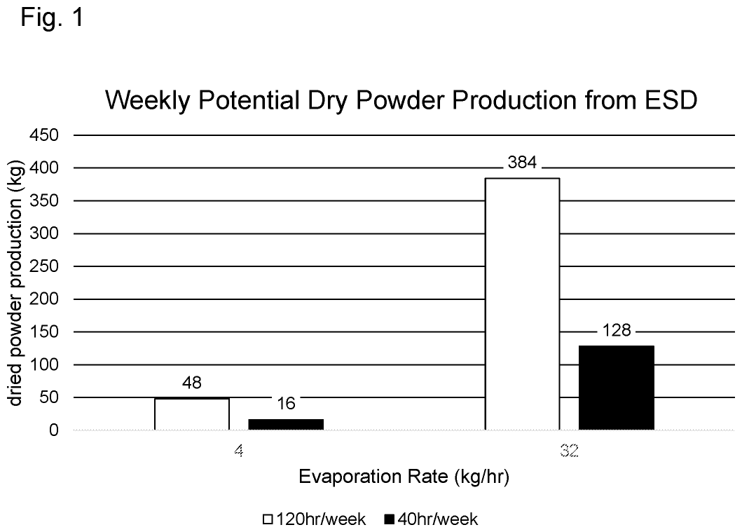

Assuming an inlet feedstock stream has 10% solids, electrospray drying with a drying capacity/evaporation rate of 4-32 kg/h can generate dried powder in the range of 16-384 kg (FIG. 1). In contrast, a freeze dryer operates in batch mode, and a freeze dryer with a drying capacity/evaporation rate of 4-32 kg/h produces 4-8 kg of dried powder per 5-day week. This presents a 4-40× difference in dry powder productivity, demonstrating another benefit to using electrospray drying.

### Example 5

Electrospray Drying with a Voltage Gradient or Oscillating Voltage Mode Will Result in an Increase in Recovery of Viable M. elsdenii Cells and Increased Shelf Life

M. elsdenii cells will be cultured and processed for electrospray drying as described in Example 2, except the voltage for charging the particles will be (1) an increasing voltage gradient from 1 (beginning of electrospray drying)-15 kV (end of electrospray drying) or (2) an oscillating voltage between 1 and 15 kV within 0. min during electrospray drying. See Tables 9 and 10, respectively. It is expected that the voltage gradient and/or oscillating voltage will increase the recovery of viable cells and shelf life of the M. elsdenii cell dried powder.

TABLE 9
Gradient Voltage for Electrospray Drying

| kV (time zero) | kV (end of ESD) |
|---|---|
| 1 | 15 |
| 2 | 14 |
| 3 | 13 |
| 4 | 12 |
| 5 | 11 |
| 6 | 10 |

TABLE 10
Oscillating Voltage for Electrospray Drying

| Lower Bound (kV) | Upper Bound (kV) | Frequency (Hz) |
|---|---|---|
| 1 | 15 | 1-500 |
| 2 | 14 | |
| 3 | 13 | |
| 4 | 12 | |
| 5 | 11 | |
| 6 | 10 | |
| 7 | 9 | |

### Example 6

Stressing Cells Prior to Electrospray Drying by Altering the Temperature, pH, or Osmolality of the Culture Will Result in an Increase in Recovery of Viable M. elsdenii Cells and Increased Shelf Life

M. elsdenii cells will be cultured and processed for electrospray drying as described in Example 2, except during the culture process, at or after mid-log growth phase, the temperature will be increased to 50-60° C. for minutes to hours then cooled to ambient temperature. It is expected that the temporary increase in temperature during culturing will cause production of stress proteins within the cells that will increase the recovery of viable cells and shelf life of the M. elsdenii cell dried powder.

M. elsdenii cells will be cultured and processed for electrospray drying as described in Example 2, except during the culture process, at or after mid-log growth phase, the pH will be increased to >7.5 or decreased to <4 for minutes to hours then returned to the original pH setpoint. It is expected that the temporary increase or decrease in pH during culturing will cause production of stress proteins within the cells that will increase the recovery of viable cells and shelf life of the M. elsdenii cell dried powder.

M. elsdenii cells will be cultured and processed for electrospray drying as described in Example 2, except during the culture process, at or after mid-log growth phase, the osmolality will be increased by adding salt to induce osmotic stress for minutes to hours then remove the additional salt to return the osmolality to the original setpoint. It is expected that the temporary increase in osmolality during culturing will cause production of stress proteins within the cells that will increase the recovery of viable cells and shelf life of the M. elsdenii cell dried powder.

### Example 7

Multiple M. elsdenii Strains can be Preserved by Electrospray Drying

To determine the effect of various electrospray drying protocols on different M. elsdenii strains, the following experiment will be performed.

The following M. elsdenii strains will be used in this experiment: (1) M. elsdenii NCIMB 41125, (2) M. elsdenii ATCC 25940, (3) M. elsdenii NCIMB 702261, (4) M. elsdenii NCIMB 702262, and (5) M. elsdenii NCIMB 702410.

The methods that will be used in this experiment are the same as described in Example 1 and 2, except as noted below. It is expected that electrospray drying M. elsdenii strains will yield similar results with regard to the amount of viable microbes recovered in the dried products post spray drying as compared to NCIMB 41125 (Example 1 and 2). Additionally, the electrospray drying process will not have an effect on microbial survivability or on the ability of the microbe to grow after being dried.

It is expected that similar to M. elsdenii NCIMB 41125, among the tested inlet temperatures (i.e. 70° C., 80° C., and 90° C.) with the same carrier (sucrose), 70° C. yielded the highest cell recovery with all other M. elsdenii strains. Increased inlet temperatures of 80° C. and 90° C. will decrease the cell yield and will not appear to significantly reduce the moisture level in the resulting powder. It is also expected that further decreasing the temperature to 65° C. and 62.5° C. will not improve the cell recovery.

It is expected that similar to M. elsdenii NCIMB 41125, among the tested carriers (i.e., sucrose, maltodextrin+maltose, and hydrophobic starch), sucrose will yield the highest cell recovery, and increases in sucrose concentrations (50-150 g/L prior to electrospray drying) will also further improve the recovery percentage.

It is expected that, similar to M. elsdenii NCIMB 41125, feedstock cell concentration (1× or 10×) will not impact cell recovery post-electrospray drying. Similarly, it is expected that feedstock cell concentrations of 22.5× and 1× will yield comparable cell recovery indicating the ability to produce a more concentrated product without impacting cell recovery efficiency.

### Example 8

### Anaerobic Bacterial Cells can be Preserved by Electrospray Drying

To determine the effect of various electrospray drying protocols on different types of anaerobic bacteria the following example will be performed.

Anaerobic bacteria can be divided into three categories, (1) obligate anaerobes; (2) aerotolerant anaerobes; and (3) facultative anaerobes. Obligate anaerobes are bacteria that do not survive in normal atmospheric concentrations of oxygen. Some obligate anaerobes can survive in up to 8% oxygen, while others cannot survive unless the oxygen concentration is less than 0.5%. Aerotolerant anaerobes can survive in the presence of oxygen, but do not utilize oxygen for growth. Facultative anaerobes are able to use oxygen for aerobic respiration but can also use anaerobic respiration if no oxygen is present.

Megasphaera, such as M. elsdenii, Fibrobacter, such as F. succinogenes, Butyrivibrio, such as B. fibrisolvens, and Bifidobacterium, such as B. breve are representative species of obligate anaerobes. Lactobacillus, such as L. plantarum and Bifidobacterium, such as B. animalis subsp. lactis are representative species of aerotolerant anaerobes. Pediococcus, such as P. acidilactici and Lactobacillus, such as L. casei are representative species of facultative anaerobes.

The methods that will be used in this experiment are the same as described in Example 1 and 2, except using the appropriate medium and growth conditions to allow for optimal microbial cell growth. It is expected that electrospray drying anaerobic bacteria (i.e., Fibrobacter succinogenes, Butyrivibrio fibrisolvens, Bifidobacterium breve, Lactobacillus plantarum, Bifidobacterium animalis subsp. lactis, Pediococcus acidilactici, Ruminococcus flavefaciens, and Lactobacillus casei) will yield similar results as seen with M. elsdenii with regard to the amount of viable cells recovered in the dried products post-electrospray drying. Additionally, the electrospray drying process will not have an effect on microbial survivability or on the ability of the microbe to grow after being dried.

It is expected that, similar to all M. elsdenii strains, among the tested inlet temperatures (i.e. 70° C., 80° C., and 90° C.) with the same carrier (sucrose), 70° C. will yield the highest cell recovery with all other anaerobic bacteria. Increased inlet temperatures of 80° C. and 90° C. will decrease the cell yield and will not appear to significantly reduce the moisture level in the resulting powder. It is also expected that further decreasing the temperature to 65° C. and 62.5° C. will not improve the cell recovery.

It is expected that, similar to all M. elsdenii strains, among the tested carriers (i.e., sucrose, maltodextrin+maltose, and hydrophobic starch), sucrose will yield the highest cell recovery, and increases in sucrose concentrations will also further improve the recovery percentage.

It is expected that, similar to all M. elsdenii strains, the feedstock cell concentration (1× or 10×) will not impact cell recovery post-electrospray drying. Similarly, it is expected that feedstock cell concentration of 22.5× and 1× will yield comparable cell recovery indicating the ability to produce a more concentrated product without impacting cell recovery efficiency.

### Example 9

Voltage Gradient During Electrospray Drying Impacts M. elsdenii Cell Recovery

Imparting voltage during spray-drying is fundamental towards electrostatic spray drying in achieving effective spray drying at a high temperature. Conventionally, ESD is conducted by holding voltage at a constant level. This example demonstrates the feasibility of ESD with pulsating voltage, while achieving effective drying and CFU recovery.

Procedure: M. elsdenii NCIMB 41125 cells were grown in semi defined media, consisting of two carbon sources, under sterile and anaerobic conditions in 500 Liter tank as described in Example 13 of WO 2018/144653 A1.

At the end of the growth phase, the cell culture was concentrated using a tangential flow filtration system (TFF) to remove 99% of the liquid (permeate) and collect a 100× retentate. Stabilizer (sucrose) and YEP solutions (sterile anaerobic diluent supplemented with 10 g/L of yeast and 5 g/L of soy peptone) were added into the 100× retentate to achieve the final cell concentration at 20× and the final additive concentrations as listed in Table 11.

TABLE 11
Final concentrations of stabilizer solution

| | Final cell | Final sucrose | |
|---|---|---|---|
| | concentrate, | concentration, | Nitrogen Source, |
| Feedstock | fold | g/L | g/L |
| 1 | 20X | 125 | 10 g/L yeast extract + |
| | | | 5 g/L peptone |

The feedstocks were electrospray dried in four runs with the following parameters in an ESD unit (PolarDry 001).

TABLE 12
Characteristics of electrospray drying process

| | Run 1 | Run 2* | Run 3* | Run 4* |
|---|---|---|---|---|
| Inlet temperature (° C.) | 80 | 80 | 80 | 80 |
| Flow rate (g/min) | 4 | 4 | 4 | 4 |
| Atomizing temperature (° C.) | 35 | 35 | 35 | 35 |
| Atomizing gas pressure (kPa) | 325 | 325 | 325 | 325 |
| Voltage (kV) | 15 kV | High: 25 kV for 3 s | High: 20 kV for 3 s | High: 20 kV for 3 s |
| | Constant | Low: 3 kV for 1 s | Low: 1 kV for 6 s | Low: 5 kV for 5 s |
| Duration (min) | 120-240 | 120-240 | 120-240 | 120-240 |

*Voltage pulsates from high to low settings throughout the ESD runs

The electrospray dried powders were collected at the end of the run, and water activity, moisture content, viability (CFU/g), and intact cells/g were determined.

CFU Analysis: Dry samples obtained post-ESD were weighed, and 0.3 g was resuspended in 60 mL of sterile anaerobic diluent or sterile anaerobic diluent supplemented with 10 g/L of yeast and 5 g/L of soy peptone. Samples were left to rehydrate for 2 hours at room temperature before being further diluted in anaerobic diluent (supplemented or not) and plated into SDL agar or enriched SDL agar with 1 mL of supplemented anaerobic diluent overnight. Plates were processed as above to determine final cell concentration in dry samples.

Cell recovery post-ESD was calculated by dividing the total amount of M. elsdenii in the dry sample by the initial total amount of M. elsdenii in the final feedstock sample (pre-ESD).

$$CFU Recovery \% = \frac{\begin{matrix}[ CFU / g ] * [ total volume spray dried ( mL ) ] * \\ [ total solids ( g ) / volume ( mL ) ] * [ \frac{dry solids}{total solids} ]\end{matrix}}{[ CFU / mL ] * [ total volume of fermentate spray dried ( mL ) ]} \times 100 \%$$

Intact Cell Analysis: BactoBox® (SBT Instruments), a portable flow cytometer, was used to measure intact cells (cells with intact lipid membrane).

Rehydrated samples from CFU analyses were serial diluted in PBS buffer (1:9 ratio) to meet the target concentration of total particles/mL of the Bactobox (10,000 to 5,000,000 total particles/mL). Samples were then analyzed with the unit and results adjusted for dilution factor and sample weight.

Intact Cell recovery post-ESD was calculated by dividing the total amount of intact M. elsdenii cells in the dry sample by the initial total intact of M. elsdenii cells in the final feedstock sample (pre-ESD).

$$Intact Cell Recovery \% = \frac{\begin{matrix}{[ \# intact cells / g ]}_{dried} * [ total volume spray dried ( mL ) ] * \\ [ total solids ( g ) / volume ( mL ) ] * [ \frac{dry solids}{total solids} ]\end{matrix}}{\begin{matrix}{[ \#intact cells / mL ]}_{feedstock} * \\ [ total volume of fermentate spray dried ( mL ) ]\end{matrix}} \times 100 \%$$

Table 13 provides the results from the ESD protocol.

TABLE 13
Results from ESD protocol

| Parameter | Run 1 | Run 2 | Run 3 | Run 4 |
|---|---|---|---|---|
| Water activity | 0.126 | 0.083 | 0.051 | 0.076 |
| Moisture Content, % | 4.31 | 2.48 | 2.79 | 3.80 |
| Viability, CFU/g | 3.5 × 10¹⁰ | 2.5 × 10¹⁰ | 2.0 × 10¹⁰ | 6.7 × 10⁹ |
| CFU Recovery, % | 20 | 14 | 11 | 4 |
| Bactobox, intact cell/g | 1.2 × 10¹¹ | 1.3 × 10¹¹ | 1.5 × 10¹¹ | 1.3 × 10¹¹ |
| Intact Cell Recovery, % | 39 | 43 | 48 | 43 |

This Example successfully demonstrated the impact of voltage pulsation on achieving ESD with a lower moisture content and water activity. A microbial powder with a lower moisture content is generally correlated with a longer shelf life as drier materials tend to be more stable. While the intact cell recovery improves with the voltage pulsation, the CFU recoveries are generally lower.

### Example 10

Electrospray Drying with Complex Nitrogen Source as a Stabilizer

This Example demonstrates the impact of utilizing a rich complex nutrient as a stabilizer to improve CFU recovery and viability.

Procedure: M. elsdenii NCIMB 41125 cells were grown in semi defined media, consisting of two carbon sources, under sterile and anaerobic conditions in a 500 liter tank at 39° C.

At the end of the growth phase, the cell culture was concentrated using a tangential flow filtration system (TFF) to remove 99% of the liquid (permeate) and collect a 100× retentate. Stabilizer and reverse osmosis (RO) water (with or without a complex nitrogen source) were added into the 100× retentate to achieve the final cell concentration at 20× and the final additive concentrations as listed in Table 14.

TABLE 14
Final additive concentrations in feedstocks

| | Final cell | Final sucrose | |
|---|---|---|---|
| | concentrate | concentration | Nitrogen Source |
| Feedstock | (fold) | (g/L) | (g/L) |
| 1 | 20 | 125 | None |
| 2 | 20 | 125 | 10 g/L yeast extract + |
| | | | 5 g/L peptone |

The feedstocks were electrospray dried with the following parameters in an ESD unit (PolarDry 001).

TABLE 15
ESD Parameters

| Parameter | Setting |
|---|---|
| Inlet temperature (° C.) | 80 |
| Flow rate (g/min) | 4 |
| Atomizing temperature (° C.) | 35 |
| Atomizing gas pressure (kPa) | 325 |
| Voltage (kV) | 15 |
| Duration (min) | 120-240 |

The electrospray dried powders were collected at the end of each run and water activity, moisture content, viability (CFU/g) and intact cells/g were determined as described in Example 9.

Table 16 provides the results from the parameters measured from the ESD protocol.

TABLE 16
Results from ESD protocol

| | Feedstock 1 | Feedstock 2 |
|---|---|---|
| | (without Complex | (with Complex |
| | Nitrogen Source) | Nitrogen Source) |
| Water activity | 0.10 | 0.08 |
| Moisture Content, % | 3.1 | 3.0 |
| Viability (CFU/g) | 3.7 × 10¹⁰ | 4.2 × 10¹⁰ |
| CFU Recovery % | 12% | 6% |
| Bactobox (intact cell/g) | 9.5 × 10¹⁰ | 1.2 × 10¹¹ |
| Intact Cell Recovery % | 31% | 40% |

These results demonstrate the feasibility and advantage of using complex nitrogen source as stabilizer during ESD to achieve higher CFU and intact cell recoveries.

### Example 11

### Accelerated and Short Term Shelf Life Study

This Example demonstrates the relative stability of dried M. elsdenii 41125 from both ESD and freeze drying (FD) process.

Procedure: The ESD powder from Example 1 was packaged and stored in metallized bags, and the head space was flushed with nitrogen gas. The bags were heat sealed and stored at −20° C., 4° C., or 37° C. Concentrated cells (20×; from the same fermentation batch in Example 1) with sucrose (50 g/L) as the stabilizer solubilized in RO water were freeze-dried. The resulting freeze-dried powder was packaged as the ESD powder and stored at 37° C. or 4° C.

The viability of the cells was analyzed periodically to gauge the stability profile at the different temperatures, following CFU analysis procedure described in Example 11. Storage temperature of 37° C. was used to mimic long term effect of storage and accelerate deterioration of the product without inducing new changes (accelerated shelf life).

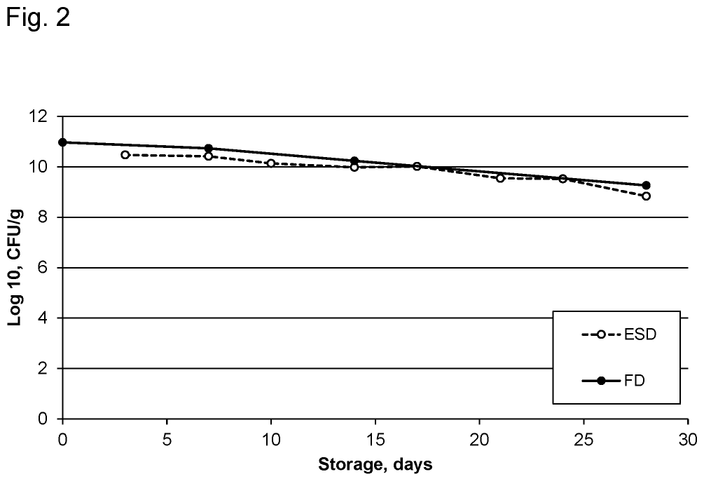

Results: FIG. 2 demonstrates that both ESD and freeze-drying produced material with a comparable stability profile under the accelerated high temperature exposure. The freeze-dried powder had higher initial CFU/g due to a lower loading of solid excipient (125 g/L sucrose vs 50 g/L sucrose). The decrease in viability of both ESD and FD powder was at a similar rate. While the stability is relatively close between the two drying technologies, ESD provide a higher throughput potential due to the continuous production mode relative to batch mode (freeze dryer).

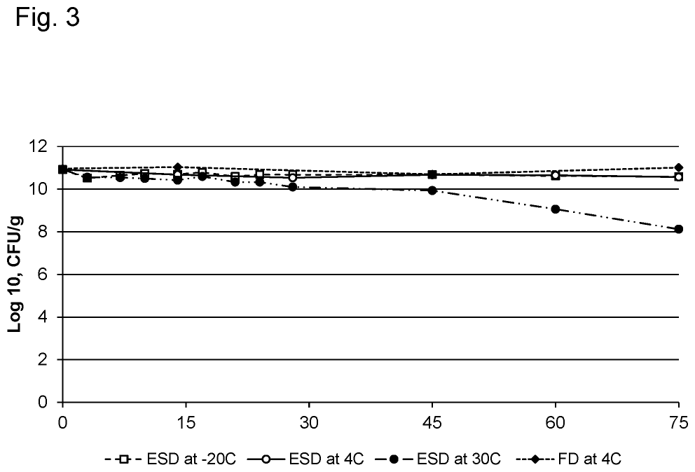

FIG. 3 demonstrates that both ESD and FD powder showed comparable decreases in viability at 4° C. In addition, ESD powder stored at −20° C. was not different from its counterpart stored at 4° C. Storage of the ESD powder at 30° C. showed a decrease in viability, especially after 30 days, when compared to its counterparts stored at −20° C. or 4° C.

### Example 12

Extended Shelf Life of Freeze-Dried M. elsdenii Produced on a Pilot Scale

This Example demonstrates the relative stability of freeze-dried M. elsdenii 41125 over an extended period of storage.

Procedure: M. elsdenii NCIMB 41125 cells were grown in semi defined media, consisting of two carbon sources, under sterile and anaerobic conditions, and freeze dried as described in Example 15 of WO 2018/144653 A1, which is incorporated by reference in its entirety.

Once freeze drying was complete, freeze-dried powder was collected and mixed with maltodextrin as a bulking agent (0.16 g of freeze-dried powder+2.34 g of maltodextrin). The resulting matrix was then packed in gelatin capsule and placed into Mylar bag while maintaining anaerobic conditions.

Twenty-seven capsules from the beginning, middle, and end of the production run were selected at random (81 capsules total). The experiment was repeated 5 times (5 production batches) for a total of 405 capsules. M. elsdenii concentration in the capsule was determined by re-suspending the freeze-dried product in the anaerobic chamber with 40 mL of anaerobic diluent, allowing it to rehydrate for 40 minutes at room temperature, and then plating onto semi defined lactate agar (i.e., viability count). M. elsdenii concentration was expressed as CFU/mL and log transformed.

Metallized bags containing the M. elsdenii capsules were stored at 4° C. Samples were obtained after 0, 0.5, 1, 3, 6, 9, 12, 18, and 24 months of storage and processed in the same way as previously described (3 samples per treatment per time point; 405 samples total) to determine M. elsdenii shelf life.

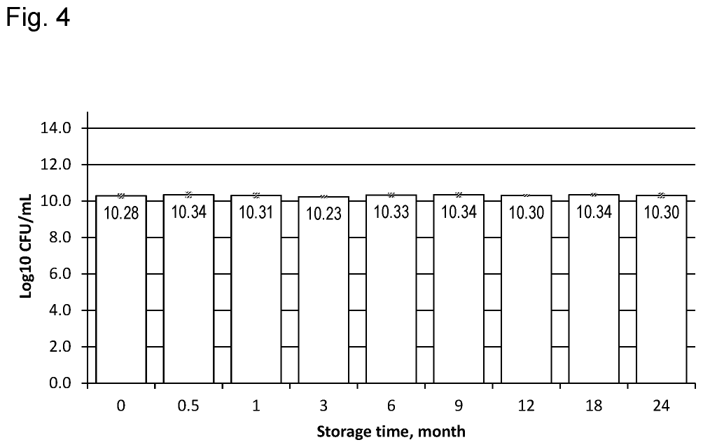

FIG. 4 demonstrates that M. elsdenii concentration was stable over time, with a 0.02 log difference between day zero sampling and 24 months sampling.

### Example 13

Extended Shelf Life of M. elsdenii Produced Using Different Inlet Temperatures During ESD

This Example demonstrates the relative stability of M. elsdenii NCIMB 41125 in ESD powder for extended period of storage regardless of the inlet temperature used during processing.

Procedure: M. elsdenii NCIMB 41125 feedstock (cell suspension) was prepared on a commercial scale, as highlighted in Example 2, by mixing the 25× cell concentrate (retentate) obtained after TFF with a 2× sucrose solution (stabilizer) to obtain a final sucrose concentration of 125 mg/mL and a cell concentration of about 22.5× compared to the initial culture. The resulting cell suspension was then spray dried using an electrostatic spray dryer as described in Example 2. The drying was achieved through heated nitrogen with a flow rate of 150 Nm³/h flow rate and an inlet temperature of 70° C., 75° C., or 80° C.

The viability of the cells was analyzed periodically to gauge the stability profile of the ESD powder processed with the different inlet temperatures. The procedure for CFU analysis was followed as described in Example 9, except that 0.3 g aliquots from each treatment were rehydrated with 30 or 40 mL sterile anaerobic diluent supplemented with 10 g/L of yeast and 5 g/L of soy peptone instead of 60 mL. Final cell concentrations were adjusted for the amount of ESD powder and log transformed. ESD powder samples were tested on month 0, 1, 2, 3, and 6. All samples were stored −20° C. throughout the shelf-life testing.

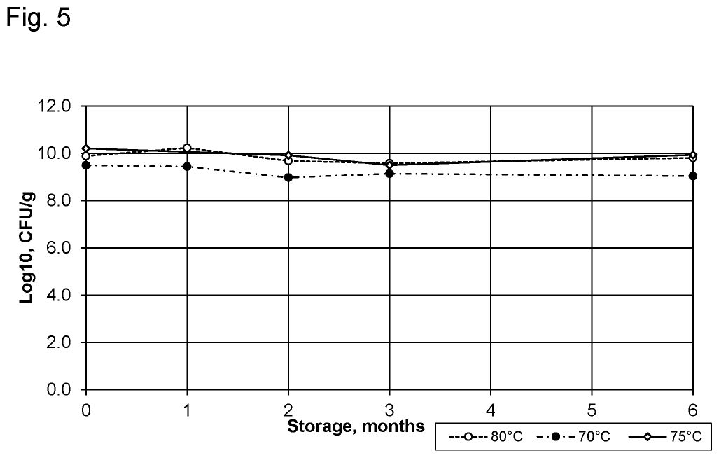

Results: FIG. 5 demonstrates that regardless of the inlet temperature used during ESD processing, all treatments resulted in stable products when stored at −20° C. for up to 6 months, confirming the accelerated shelf-life results.

M. elsdenii concentration was stable over time, with a 0.08, 0.27, and 0.45 log difference between day zero sampling and 6 months sampling for the 80° C., 75° C., and 70° C. inlet temperatures, respectively. These results are not different from the results observed in Example 3 for FD M. elsdenii products.

### Example 14

ESD with Encapsulating Polymers

This Example demonstrates the feasibility to spray-dry and encapsulate strict anaerobes in one step while achieving relatively high CFU and intact cell recoveries.

Procedure: M. elsdenii NCIMB 41125 cells were grown in semi defined media, consisting of two carbon sources, under sterile and anaerobic conditions in a 500 liter tank at 39° C.

At the end of the growth phase, the cell culture was concentrated using a tangential flow filtration system (TFF) to remove 99% of the liquid (permeate) and collect a 100× retentate. Stabilizer and encapsulating polymer (alginate or gum arabic), and RO water were added into the 100× retentate to achieve the final cell concentration of 20× and the final additive concentrations as listed in Table 17.

TABLE 17
Final additive concentrations in feedstocks

| | Final cell | Final sucrose | Final encapsulating |
|---|---|---|---|
| | concentrate, | concentration, | polymer concentration, |
| Feedstock | fold | g/L | % |
| 1 | 20X | 125 | 1.0% low viscosity alginate |
| 2 | 20X | 125 | 2.0% gum arabic |

The feedstocks were electrospray dried with the parameters in Table 18 in an ESD unit (PolarDry 001).

TABLE 18
ESD Parameters

| Parameter | Setting |
|---|---|
| Inlet temperature, ° C. | 80 |
| Flow rate, g/min | 4 |
| Atomizing temperature, ° C. | 35 |
| Atomizing gas pressure, kPa | 325 |
| Voltage, kV | 15 |
| Duration, min | 120-240 |

The electrospray dried powders were collected at the end of the run, and water activity, moisture content, viability (CFU/g), and intact cells/g were determined.

The viability of the cells was analyzed periodically to gauge the stability profile at the different temperatures, following CFU analysis and intact cell analysis procedure described in Example 9.

Table 19 provides the results from the parameters measured from the ESD protocol.

TABLE 19
Results from ESD protocol

| | Feedstock 1 (with low | Feedstock 2 (with |
|---|---|---|
| Parameter | viscosity alginate) | gum arabic) |
| Water activity | 0.089 | 0.093 |
| Moisture Content, % | 3.73 | 3.18 |
| Viability, CFU/g | 1.1 × 10¹⁰ | 2.2 × 10¹⁰ |
| CFU Recovery, % | 6 | 12 |
| Bactobox, intact cell/g | 9.5 × 10¹⁰ | 9.5 × 10¹⁰ |
| Intact Cell Recovery, % | 54 | 54 |

These results demonstrate the feasibility of encapsulating microbes within the soluble excipients with an encapsulating polymer in one step during ESD, and maintaining high CFU and intact cell recovery.

### Example 15

ESD with a Mixture of Sugar and Sugar Alcohol

Sugar alcohols, such as sorbitol, xylitol, mannitol, are known as an osmolyte protectant molecule. However, it is unclear whether it is effective as a protectant molecule for drying vegetative cells. This result demonstrates its feasibility.

Procedure: M. elsdenii NCIMB 41125 cells were grown in semi defined media, consisting of two carbon sources, under sterile and anaerobic conditions in a 500 liter tank at 39° C.

At the end of the growth phase, the cell culture was concentrated using a tangential flow filtration system (TFF) to remove 99% of the liquid (permeate) and collect a 100× retentate. Stabilizers (sucrose+sorbitol) and YEP solution (sterile anaerobic diluent supplemented with 10 g/L of yeast and 5 g/L of soy peptone) were added into the 100× retentate to achieve the final cell concentration at 20× and the final additive concentrations as listed in Table 20.

TABLE 20
Final additive concentrations in the feedstock

| | Final cell | Final sucrose | Final sorbitol | Nitrogen |
|---|---|---|---|---|
| Feed- | concentrate, | concentration, | concentration, | Source, |
| stock | fold | g/L | g/L | g/L |
| 1 | 20X | 93.75 | 31.25 | 10 g/L yeast |
| | | | | extract + |
| | | | | 5 g/L peptone |

The feedstock was electrospray dried with the following parameters in an ESD unit (PolarDry 001).

TABLE 21
ESD Parameters

| Parameter | Setting |
|---|---|
| Inlet temperature, ° C. | 80 |
| Flow rate, g/min | 4 |
| Atomizing temperature, ° C. | 35 |
| Atomizing gas pressure, kPa | 325 |
| Voltage, kV | 15 |
| Duration, min | 120-240 |

The electrospray dried powders were collected at the end of the run, and water activity, moisture content, viability (CFU/g), and intact cells/g were determined. CFU analyses and intact cells analysis were performed as described in Example 9.

Table 22 provides the results from the parameters measured from the ESD protocol.

TABLE 22
Results from ESD protocol

| Parameter | Results |
|---|---|
| Water activity | 0.035 |
| Moisture Content, % | 1.67 |
| Viability, CFU/g | 3.4 × 10¹⁰ |
| CFU Recovery % | 19 |
| Bactobox, intact cell/g | 1.5 × 10¹¹ |
| Intact Cell Recovery, % | 86 |

This example successfully demonstrated the formulation is able to yield high CFU recovery and intact cell recovery.

### Example 16

ESD with Gram-Positive Strict Anaerobe (Ruminococcus flavefaciens)

This example demonstrates the applicability of the ESD process for another strict anaerobe from a different category classified by its cell wall characteristics. Rumminococcus flavefaciens is also a strict anaerobe (like M. elsdenii), however, it is gram-positive. The efficacy of a spray-drying process is closely related to its cell wall structure and integrity. Therefore, having an effective ESD process demonstrated in both gram-positive and gram-negative microorganisms are key indicators of its range of applications.

Procedure: Rumminococcus flavefaciens ATCC 49949 cells were grown in a semi defined media, consisting of two carbon sources, under sterile and anaerobic conditions in 1 L pyrex bottles with tri-port attachments. The inoculated media was incubated at 37° C. for 12 hours in an anaerobic chamber.

At the end of the growth phase, the stabilizer (sucrose) was added into the fermentate to achieve the final additive concentration listed in Table 23.

TABLE 23
Final additive concentrations in the feedstock

| | Final cell | Final sucrose | |
|---|---|---|---|
| Feedstock | concentrate, fold | concentration, g/L | Nitrogen Source, g/L |
| 1 | 1 (no | 125 | 10 g/L yeast extract + |
| | concentration) | | 5 g/L peptone |

The feedstock was electrospray dried with the following parameters in an ESD unit (PolarDry 001).

TABLE 24
ESD Parameters

| Parameter | Setting |
|---|---|
| Inlet temperature, ° C. | 80 |
| Flow rate, g/min | 4 |
| Atomizing temperature, ° C. | 35 |
| Atomizing gas pressure, kPa | 325 |
| Voltage, kV | 15 (Constant) |
| Duration, min | 120-240 |

The electrospray dried powders were collected at the end of the run, and water activity, moisture content, viability (CFU/g), and intact cells/g were determined. CFU analysis and intact cell analysis were performed according to the procedure described in Example 9.

Table 25 provides the results from the parameters measured from the ESD protocol.

TABLE 25
Results from ESD protocol

| Parameter | Results |
|---|---|
| Water activity | 0.1 |
| Moisture Content, % | 3.5 |
| Viability, CFU/g | 9.0 × 10⁸ |
| CFU Recovery, % | 143 |
| Bactobox, intact cell/g | 1.2 × 10⁹ |
| Intact Cell Recovery, % | 141 |

These results indicated virtually complete 100% CFU recovery and intact cell recovery from ESD process on Ruminococcus flavefaciens ATCC 49949 cells. Ruminococcus flavefaciens presented higher recoveries compared to M. elsdenii NCIMB 41125. In view of the overall results from both strict anaerobes (M. elsdenii, gram-negative microbe; R. flavefaciens, gram-positive microbe), the described ESD process was shown to be effective to stabilize and recover high levels of viable anaerobic cells having different characteristics.

What is claimed is:

1. A system for spray drying Megasphaera elsdenii cells into a dried powder, the system comprising:
a source of water, a carrier, and Megasphaera elsdenii cells;
a tank comprising a stirring device and the tank is arranged to receive the water, the carrier, and the Megasphaera elsdenii cells from the source to form a slurry having a viscosity in the range of about 1 cP to about 500 cP;
an electrode configured to apply an electrostatic charge to the slurry using a voltage gradient or oscillating voltages;
an atomizer;
a drying chamber comprising an inlet end, an outlet end, and an internal volume located between the inlet end and the outlet end, the internal volume being configured to hold the slurry and a drying fluid,
wherein the drying chamber is configured to dry the slurry;
wherein the atomizer is configured to:
(i) receive the slurry from the tank; and
(ii) discharge the slurry into the drying chamber for contact with the drying fluid to form the dried powder containing the Megasphaera elsdenii cells; and
wherein the drying fluid comprises nitrogen or argon at a temperature between about 50° C. to about 100° C., and wherein the entire system is under less than 2% oxygen.

2. A method of electrospray drying Megasphaera elsdenii cells into a dried powder, comprising:
preparing a culture comprising Megasphaera elsdenii cells, a growth media, and an osmolyte protectant molecule;
harvesting the Megasphaera elsdenii cells;
forming a slurry comprising water, a carrier, and the Megasphaera elsdenii cells, wherein the carrier has a final concentration of about 1% to about 50% wt and the slurry has a viscosity in the range of about 1 cP to about 500 cP;
applying an electrostatic charge to the slurry using a voltage gradient or oscillating voltages;
atomizing the slurry to generate a spray of liquid droplets of the slurry;
introducing the spray of liquid droplets of the slurry into a drying chamber; and
feeding a drying fluid at a temperature between about 50° C. to about 100° C. into the drying chamber to dry the liquid droplets to form the dried powder comprising a plurality of dried particles containing the Megasphaera elsdenii cells encapsulated within the carrier, wherein the dried powder comprises less than 15% moisture content, and wherein the entire method is performed under less than 2% oxygen.

3. A method of electrospray drying Megasphaera elsdenii cells into a dried powder, comprising:
preparing a culture comprising Megasphaera elsdenii cells, a growth media, and an osmolyte protectant molecule, wherein during the culture the pH, temperature, and/or osmolality are altered for a time period;
harvesting the Megasphaera elsdenii cells;
forming a slurry comprising water, a carrier, and the Megasphaera elsdenii cells, wherein the carrier has a final concentration of about 1% to about 50% wt and the slurry has a viscosity in the range of about 1 cP to about 500 cP;
applying an electrostatic charge to the slurry using a voltage gradient or oscillating voltages;
atomizing the slurry to generate a spray of liquid droplets of the slurry;
introducing the spray of liquid droplets of the slurry into a drying chamber; and
feeding a drying fluid at a temperature between about 50° C. to about 100° C. into the drying chamber to dry the liquid droplets to form the dried powder comprising a plurality of dried particles containing the Megasphaera elsdenii cells encapsulated within the carrier, wherein the dried powder comprises less than 15% moisture content, and wherein the entire method is performed under less than 2% oxygen.

4. A method of electrospray drying Megasphaera elsdenii cells into a dried powder, comprising:
preparing a culture comprising Megasphaera elsdenii cells, and a growth media;
adding an osmolyte protectant molecule;
harvesting the Megasphaera elsdenii cells;
forming a slurry comprising water, a carrier, and the Megasphaera elsdenii cells, wherein the carrier has a final concentration of about 1% to about 50% wt and the slurry has a viscosity in the range of about 1 cP to about 500 cP;
applying an electrostatic charge to the slurry using a voltage gradient or oscillating voltages;
atomizing the slurry to generate a spray of liquid droplets of the slurry;
introducing the spray of liquid droplets of the slurry into a drying chamber; and
feeding a drying fluid at a temperature between about 50° C. to about 100° C. into the drying chamber to dry the liquid droplets to form the dried powder comprising a plurality of dried particles containing the Megasphaera elsdenii cells encapsulated within the carrier, wherein the dried powder comprises less than 15% moisture content, and wherein the entire method is performed under less than 2% oxygen.

5. A method of electrospray drying Megasphaera elsdenii cells into a dried powder, comprising:
preparing a culture comprising Megasphaera elsdenii cells, and a growth media, wherein during the culture the pH, temperature, and/or osmolality are altered for a time period;
adding an osmolyte protectant molecule;
harvesting the Megasphaera elsdenii cells;
forming a slurry comprising water, a carrier, and the Megasphaera elsdenii cells, wherein the carrier has a final concentration of about 1% to about 50% wt and the slurry has a viscosity in the range of about 1 cP to about 500 cP;
applying an electrostatic charge to the slurry using a voltage gradient or oscillating voltages;
atomizing the slurry to generate a spray of liquid droplets of the slurry;
introducing the spray of liquid droplets of the slurry into a drying chamber; and
feeding a drying fluid at a temperature between about 50° C. to about 100° C. into the drying chamber to dry the liquid droplets to form the dried powder comprising a plurality of dried particles containing the Megasphaera elsdenii cells encapsulated within the carrier, wherein the dried powder comprises less than 15% moisture content, and wherein the entire method is performed under less than 2% oxygen.

6. A method of electrospray drying Megasphaera elsdenii cells into a dried powder, comprising:
preparing a culture comprising Megasphaera elsdenii cells, and a growth media;
harvesting the Megasphaera elsdenii cells;
adding an osmolyte protectant molecule;
forming a slurry comprising water, a carrier, and the Megasphaera elsdenii cells, wherein the carrier has a final concentration of about 1% to about 50% wt and the slurry has a viscosity in the range of about 1 cP to about 500 cP;
applying an electrostatic charge to the slurry using a voltage gradient or oscillating voltages;
atomizing the slurry to generate a spray of liquid droplets of the slurry;
introducing the spray of liquid droplets of the slurry into a drying chamber; and
feeding a drying fluid at a temperature between about 50° C. to about 100° C. into the drying chamber to dry the liquid droplets to form the dried powder comprising a plurality of dried particles containing the Megasphaera elsdenii cells encapsulated within the carrier, wherein the dried powder comprises less than 15% moisture content, and wherein the entire method is performed under less than 2% oxygen.

7. A method of electrospray drying Megasphaera elsdenii cells into a dried powder, comprising:
preparing a culture comprising Megasphaera elsdenii cells, and a growth media, wherein during the culture the pH, temperature, and/or osmolality are altered for a time period;
harvesting the Megasphaera elsdenii cells;
adding an osmolyte protectant molecule;
forming a slurry comprising water, a carrier, and the Megasphaera elsdenii cells, wherein the carrier has a final concentration of about 1% to about 50% wt and the slurry has a viscosity in the range of about 1 cP to about 500 cP;
applying an electrostatic charge to the slurry using a voltage gradient or oscillating voltages;
atomizing the slurry to generate a spray of liquid droplets of the slurry;
introducing the spray of liquid droplets of the slurry into a drying chamber; and
feeding a drying fluid at a temperature between about 50° C. to about 100° C. into the drying chamber to dry the liquid droplets to form the dried powder comprising a plurality of dried particles containing the Megasphaera elsdenii cells encapsulated within the carrier, wherein the dried powder comprises less than 10% moisture content, and wherein the entire method is performed under less than 2% oxygen.

8. A method of forming a dry powder comprising Megasphaera elsdenii cells encapsulated within a carrier, said method comprising:
forming a slurry in a mixing tank comprising water, a carrier, and Megasphaera elsdenii cells while stirring the mixing tank with the stirring device;
applying an electrostatic charge to the slurry using a voltage gradient or oscillating voltages;
atomizing the slurry in an atomizer and discharging atomized slurry into a drying chamber for contact with a drying fluid to form the dried powder containing the Megasphaera elsdenii cells encapsulated within the carrier, and wherein the drying fluid comprises nitrogen or argon at a temperature between about 50° C. to about 100° C., and wherein the dried powder comprises less than 15% moisture content, and wherein the entire method is performed under less than 2% oxygen.

9. The method of any one of claims 2-7, wherein the Megasphaera elsdenii cells are concentrated about 20× to about 100× prior to harvesting.

10. The method of any one of claims 2-8, further comprising heating the drying fluid to between about 50° C. to about 100° C. by applying a voltage of about 0.1 kV to about 45 kV prior to feeding the drying fluid into the drying chamber.

11. The method of any one of claims 2-10, further comprising heating the drying fluid to between about 50° C. to about 100° C. by applying a voltage of about 10 kV to about 30 kV prior to feeding the drying fluid into the drying chamber.

12. The method of any one of claims 2-8, wherein the drying fluid is at a temperature between about 60° C. to about 90° C.

13. The method of claim 12, further comprising heating the drying fluid to between about 60° C. to about 90° C. by applying a voltage of about 10 kV to about 30 kV prior to feeding the drying fluid into the drying chamber.

14. The method of any one of claims 10, 11, and 13, wherein the voltage is applied constantly or in pulses.

15. The method of any one of claims 2-14, wherein the carrier is selected from the group consisting of a sucrose, a sugar, a sugar alcohol, a sugar derivative, a polysaccharide, an encapsulating polymer, or a nitrogen source, and a mixture thereof.

16. The method of claim 15, wherein the carrier is present in an amount of about 1% to about 40% (w/v).

17. The method of any one of claims 2-16, wherein the dried powder comprises about 1×10³ to about 1×10¹³ CFU/gram of Megasphaera elsdenii cells.

18. The method of claim 17, wherein the dried powder comprises about 1×10³ CFU/gram of Megasphaera elsdenii cells.

19. The method of any one of claims 1 to 18, wherein the volume of the culture is at least 50 liters.

20. A feed additive comprising the electrospray dried M. elsdenii cells produced by the method of any one of claims 1 to 19.

21. The feed additive of claim 20, wherein the feed additive further comprises another microorganism.

22. The feed additive of claim 20 or 21, wherein the feed additive is selected from the group consisting of: a powder, a granulate, a particulate, a pellet, a cake, or combinations thereof.

23. The feed additive of any one of claims 20 to 22, wherein the feed additive is a probiotic.

24. A composition comprising the electrospray dried M. elsdenii cells produced by the method of any one of claims 1-19 or the feed additive of any one of claims 20 to 23.

25. The composition of claim 24, wherein the composition is a capsule.

26. A kit comprising the electrospray dried M. elsdenii cells produced by the method of any one of claims 1 to 19, the feed additive of any one of claims 20 to 23, or the composition of claim 24 or 25.

27. A method for treating or preventing a condition or disorder associated with lactic acid production in the gastrointestinal tract of an animal, comprising administering to the animal an effective amount of the electrospray dried M. elsdenii cells produced by the method of any one of claims 1 to 19, the feed additive of any one of claims 20 to 23, or the composition of claim 24 or 25.

28. The method of claim 27, wherein the condition or disorder is acidosis.

29. The method of claim 27 or 28, wherein the condition or disorder is ruminal acidosis.

30. The method of claim 27, wherein the condition or disorder is respiratory disease.

31. The method of claim 27, wherein the condition or disorder is laminitis.

32. The method of claim 27, wherein the condition or disorder is an infection.

33. The method of claim 32, wherein the infection is caused by Salmonella or Campylobacter.

34. A method for preventing or decreasing the growth of an opportunistic microorganism in the gastrointestinal tract of an animal, comprising administering to the animal an effective amount of the electrospray dried M. elsdenii cells produced by the methods of any one of claims 1 to 19, the feed additive of any one of claims 20 to 23, or the composition of claim 24 or 25.

35. The method of claim 34, wherein the opportunistic microorganism is pathogenic.

36. The method of claim 34 or 35, wherein the opportunistic microorganism is Salmonella or Campylobacter.

37. A method of improving the bioavailability of plant-derived phosphorous in the diet of an animal, comprising administering to the animal an effective amount of the electrospray dried M. elsdenii cells produced by the methods of any one of claims 1 to 19, the feed additive of any one of claims 20 to 23, or the composition of claim 24 or 25.

38. A method of improving growth performance in an animal, comprising administering to the animal an effective amount of the electrospray dried M. elsdenii cells produced by the methods of any one of claims 1 to 19, the feed additive of any one of claims 20 to 23, or the composition of claim 24 or 25, wherein the improved growth performance in the animal is an improvement in: feed intake, average daily gain, feed conversion ratio, carcass gain, milk production in a milk-producing animal, egg production in poultry, bone mineralization, or combinations thereof.

39. The method of any one of claims 27 to 38, wherein the electrospray dried M. elsdenii cells, the feed additive, or the composition is administered prior to, concomitantly with, or after feeding the animal with a food.

40. The method of any one of claims 27 to 38, wherein the method further comprises mixing the electrospray dried M. elsdenii cells or the feed additive with a liquid prior to administration.

41. The method of claim 40, wherein the liquid is administered orally or by spraying the animal with the liquid.

42. The method of any one of claims 27 to 41, comprising a single administration of the electrospray dried M. elsdenii cells, feed additive, or composition.

43. The method of any one of claims 27 to 42, comprising a daily administration of the electrospray dried M. elsdenii cells, feed additive, or composition.

44. The method of any one of claims 27 to 43, comprising more than one administration of the electrospray dried M. elsdenii cells, feed additive, or composition on a single day.

45. The method of any one of claims 27 to 44, wherein the animal is a ruminant.

46. The method of claim 45, wherein the ruminant is selected from the group consisting of: cattle, sheep, goats, deer, buffalo, and reindeer.

47. The method of any one of claims 27 to 44, wherein the animal is a non-ruminant.

48. The method of claim 47, wherein the non-ruminant is selected from the group consisting of: equine, poultry animal, and swine.

49. The method of claim 48, wherein the poultry animal is selected from the group consisting of: a chicken, a goose, a duck, a quail, a turkey, or a pigeon.

50. The method of claim 48 or 49, wherein the poultry animal is selected from the group consisting of: a broiler, a broiler breeder, and a layer.

51. The method of any one of claims 48 to 50, wherein the poultry animal is a chicken.

52. The method of claim 48, wherein the equine is a horse, a pony, a donkey, or a mule.

53. The system of claim 1, wherein the carrier is selected from the group consisting of a sucrose, a sugar, a sugar alcohol, polysaccharide, an encapsulating polymer, a sugar derivative, or a nitrogen source, and a mixture thereof.

54. The system of claim 1, wherein the dried powder comprises about 1×10³ to about 1×10¹³ CFU/gram of Megasphaera elsdenii cells.

55. The system of claim 1, wherein the dried powder comprises about 1×10³ CFU/gram of Megasphaera elsdenii cells.

56. A dried powder, comprising a plurality of dried particles formed by the method of any one of claims 2-19.

57. Megasphaera elsdenii cells encapsulated within a carrier, prepared by the method according to any one of claims 2-19.

58. A system for spray drying anaerobic cells into a dried powder, the system comprising: a source of water, a carrier, and anaerobic cells;
a tank comprising a stirring device and the tank is arranged to receive the water, the carrier, and the anaerobic cells from the source to form a slurry having a viscosity in the range of about 1 cP to about 500 cP;
an electrode configured to apply an electrostatic charge to the slurry using a voltage gradient or oscillating voltages;
an atomizer;
a drying chamber comprising an inlet end, an outlet end, and an internal volume located between the inlet end and the outlet end, the internal volume being configured to hold the slurry and a drying fluid,
wherein the drying chamber is configured to dry the slurry;
wherein the atomizer is configured to:
(i) receive the slurry from the tank; and
(ii) discharge the slurry into the drying chamber for contact with the drying fluid to form the dried powder containing the anaerobic cells; and
wherein the drying fluid comprises nitrogen or argon at a temperature between about 50° C. to about 100° C., and wherein the entire system is under less than 2% oxygen.

59. A method of electrospray drying anaerobic cells into a dried powder, comprising:
preparing a culture comprising anaerobic cells, a growth media, and an osmolyte protectant molecule;
harvesting the anaerobic cells;
forming a slurry comprising water, a carrier, and the anaerobic cells, wherein the carrier has a final concentration of about 1% to about 50% wt and the slurry has a viscosity in the range of about 1 cP to about 500 cP;
applying an electrostatic charge to the slurry using a voltage gradient or oscillating voltages;
atomizing the slurry to generate a spray of liquid droplets of the slurry;
introducing the spray of liquid droplets of the slurry into a drying chamber; and
feeding a drying fluid at a temperature between about 50° C. to about 100° C. into the drying chamber to dry the liquid droplets to form the dried powder comprising a plurality of dried particles containing the anaerobic cells encapsulated within the carrier, wherein the dried powder comprises less than 15% moisture content, and wherein the entire method is performed under less than 2% oxygen.

60. A method of electrospray drying anaerobic cells into a dried powder, comprising:
preparing a culture comprising anaerobic cells, a growth media, and an osmolyte protectant molecule, wherein during the culture the pH, temperature, and/or osmolality are altered for a time period;
harvesting the anaerobic cells;
forming a slurry comprising water, a carrier, and the anaerobic cells, wherein the carrier has a final concentration of about 1% to about 50% wt and the slurry has a viscosity in the range of about 1 cP to about 500 cP;
applying an electrostatic charge to the slurry using a voltage gradient or oscillating voltages;
atomizing the slurry to generate a spray of liquid droplets of the slurry;
introducing the spray of liquid droplets of the slurry into a drying chamber; and
feeding a drying fluid at a temperature between about 50° C. to about 10° C. into the drying chamber to dry the liquid droplets to form the dried powder comprising a plurality of dried particles containing the anaerobic cells encapsulated within the carrier, wherein the dried powder comprises less than 15% moisture content, and wherein the entire method is performed under less than 2% oxygen.

61. A method of electrospray drying anaerobic cells into a dried powder, comprising:
preparing a culture comprising anaerobic cells, and a growth media;
adding an osmolyte protectant molecule;
harvesting the anaerobic cells;
forming a slurry comprising water, a carrier, and the anaerobic cells, wherein the carrier has a final concentration of about 1% to about 50% wt and the slurry has a viscosity in the range of about 1 cP to about 500 cP;
applying an electrostatic charge to the slurry using a voltage gradient or oscillating voltages;
atomizing the slurry to generate a spray of liquid droplets of the slurry;
introducing the spray of liquid droplets of the slurry into a drying chamber; and
feeding a drying fluid at a temperature between about 50° C. to about 100° C. into the drying chamber to dry the liquid droplets to form the dried powder comprising a plurality of dried particles containing the anaerobic cells encapsulated within the carrier, wherein the dried powder comprises less than 15% moisture content, and wherein the entire method is performed under less than 2% oxygen.

62. A method of electrospray drying anaerobic cells into a dried powder, comprising:
preparing a culture comprising anaerobic cells, and a growth media, wherein during the culture the pH, temperature, and/or osmolality are altered for a time period;
adding an osmolyte protectant molecule;
harvesting the anaerobic cells;
forming a slurry comprising water, a carrier, and the anaerobic cells, wherein the carrier has a final concentration of about 1% to about 50% wt and the slurry has a viscosity in the range of about 1 cP to about 500 cP;
applying an electrostatic charge to the slurry using a voltage gradient or oscillating voltages;
atomizing the slurry to generate a spray of liquid droplets of the slurry;
introducing the spray of liquid droplets of the slurry into a drying chamber; and
feeding a drying fluid at a temperature between about 50° C. to about 100° C. into the drying chamber to dry the liquid droplets to form the dried powder comprising a plurality of dried particles containing the anaerobic cells encapsulated within the carrier, wherein the dried powder comprises less than 15% moisture content, and wherein the entire method is performed under less than 2% oxygen.

63. A method of electrospray drying anaerobic cells into a dried powder, comprising:
preparing a culture comprising anaerobic cells, and a growth media;
harvesting the anaerobic cells;
adding an osmolyte protectant molecule;
forming a slurry comprising water, a carrier, and the anaerobic cells, wherein the carrier has a final concentration of about 1% to about 50% wt and the slurry has a viscosity in the range of about 1 cP to about 500 cP;
applying an electrostatic charge to the slurry using a voltage gradient or oscillating voltages;
atomizing the slurry to generate a spray of liquid droplets of the slurry;
introducing the spray of liquid droplets of the slurry into a drying chamber; and
feeding a drying fluid at a temperature between about 50° C. to about 100° C. into the drying chamber to dry the liquid droplets to form the dried powder comprising a plurality of dried particles containing the anaerobic cells encapsulated within the carrier, wherein the dried powder comprises less than 15% moisture content, and wherein the entire method is performed under less than 2% oxygen.

64. A method of electrospray drying anaerobic cells into a dried powder, comprising:
preparing a culture comprising anaerobic cells, and a growth media, wherein during the culture the pH, temperature, and/or osmolality are altered for a time period;
harvesting the anaerobic cells;
adding an osmolyte protectant molecule;
forming a slurry comprising water, a carrier, and the anaerobic cells, wherein the carrier has a final concentration of about 1% to about 50% wt and the slurry has a viscosity in the range of about 1 cP to about 500 cP;
applying an electrostatic charge to the slurry using a voltage gradient or oscillating voltages;
atomizing the slurry to generate a spray of liquid droplets of the slurry;
introducing the spray of liquid droplets of the slurry into a drying chamber; and
feeding a drying fluid at a temperature between about 50° C. to about 100° C. into the drying chamber to dry the liquid droplets to form the dried powder comprising a plurality of dried particles containing the anaerobic cells encapsulated within the carrier, wherein the dried powder comprises less than 15% moisture content, and wherein the entire method is performed under less than 2% oxygen.

65. A method of forming a dry powder comprising anaerobic cells encapsulated within a carrier, said method comprising:
forming a slurry in a mixing tank comprising water, a carrier, and anaerobic cells while stirring the mixing tank with the stirring device;
applying an electrostatic charge to the slurry using a voltage gradient or oscillating voltages;
atomizing the slurry in an atomizer and discharging atomized slurry into a drying chamber for contact with a drying fluid to form the dried powder containing the anaerobic cells encapsulated within the carrier, and wherein the drying fluid comprises nitrogen or argon at a temperature between about 50° C. to about 100° C., and wherein the dried powder comprises less than 15% moisture content, and wherein the entire method is performed under less than 2% oxygen.

66. The method of any one of claims 59-64, wherein the anaerobic cells are concentrated about 20× to about 100× prior to harvesting.

67. The method of any one of claims 59-66, further comprising heating the drying fluid to between about 50° C. to about 100° C. by applying a voltage of about 0.1 kV to about 45 kV prior to feeding the drying fluid into the drying chamber.

68. The method of any one of claims 59-67, further comprising heating the drying fluid to between about 50° C. to about 100° C. by applying a voltage of about 10 kV to about 30 kV prior to feeding the drying fluid into the drying chamber.

69. The method of any one of claims 59-65, wherein the drying fluid is at a temperature between about 60° C. to about 90° C.

70. The method of claim 69, further comprising heating the drying fluid to between about 60° C. to about 90° C. by applying a voltage of about 10 kV to about 30 kV prior to feeding the drying fluid into the drying chamber.

71. The method of any one of claims 67, 68, and 70, wherein the voltage is applied constantly or in pulses.

72. The method of any one of claims 59-71, wherein the carrier is selected from the group consisting of a sucrose, a sugar, a sugar alcohol, polysaccharide, an encapsulating polymer, a sugar derivative, or a nitrogen source, and a mixture thereof.

73. The method of any one of claims 59-72, wherein the dried powder comprises about 1×10³ to about 1×10¹³ CFU/gram of anaerobic cells.

74. The method of claim 73, wherein the dried powder comprises about 1×10³ CFU/gram of anaerobic cells.

75. The system of claim 58, wherein the carrier is selected from the group consisting of a sucrose, a sugar, a sugar alcohol, a sugar derivative, a polysaccharide, an encapsulating polymer, or a nitrogen source, and a mixture thereof.

76. The system of claim 58, wherein the dried powder comprises about 1×10³ to about 1×10¹³ CFU/gram of anaerobic cells.

77. The system of claim 58, wherein the dried powder comprises about 1×10³ CFU/gram of anaerobic cells.

78. A dried powder, comprising a plurality of dried particles formed by the method of any one of claims 59-74.

79. Anaerobic cells encapsulated within a carrier, prepared by the method according to any one of claims 59-74.

80. A method of improving growth performance in an animal, comprising administering to the animal an effective amount of electrospray dried anaerobic cells produced by the method of any one of claims 59-74, wherein the improved growth performance in the animal is an improvement in: feed intake, average daily gain, feed conversion ratio, carcass gain, milk production in a milk-producing animal, egg production in poultry, bone mineralization, or combinations thereof.

81. A method for preventing or decreasing the growth of an opportunistic microorganism in the gastrointestinal tract of an animal, comprising administering to the animal an effective amount of electrospray dried anaerobic cells produced by the method of any one of claims 59-74.

82. A composition comprising electrospray dried anaerobic bacterial cells produced by the method of any one of claims 59-74.

83. The method of any one of claims 2-7, wherein the Megasphaera elsdenii cells are grown at about 30° C. to about 40° C., at about 30° C., at about 31° C., at about 32° C., at about 33° C., at about 34° C., at about 35° C., at about 36° C., at about 37° C., at about 38° C., at about 39° C., or at about 40° C. prior to the harvesting.

84. The method of any one of claims 59-64, wherein the anaerobic cells are grown at about 30° C. to about 40° C., at about 30° C., at about 31° C., at about 32° C., at about 33° C., at about 34° C., at about 35° C., at about 36° C., at about 37° C., at about 38° C., at about 39° C., or at about 40° C. prior to the harvesting.

85. The method of any one of claims 2-7, wherein the carrier has a final concentration of about 2% to about 30% wt.

86. The method of any one of claims 59-64, wherein the carrier has a final concentration of about 2% to about 30% wt.

87. The method of any one of claims 1-7, 9-19, 59-64, 66-74 and 83-86, wherein the drying fluid is selected from the group consisting of nitrogen and argon.

88. The method of any one of claims 2-7, further comprising heating the drying fluid to between about 50° C. to about 100° C. by applying a voltage of about 11 kV to about 25 kV prior to feeding the drying fluid into the drying chamber.

89. The method of any one of claims 59-64, further comprising heating the drying fluid to between about 50° C. to about 100° C. by applying a voltage of about 11 kV to about 25 kV prior to feeding the drying fluid into the drying chamber.

90. The method of claim 15, wherein the encapsulating polymer is alginate.

91. The method of claim 72 or 75, wherein the encapsulating polymer is alginate.

92. The method of any one of claims 1-7, 9-19, 59-64, 66-74 and 83-86, wherein the slurry is processed at a rate of between about 1 L/hour and about 10,000 L/hour.
